SEQUENCE LISTING (13 sequences)

SEQ ID NO: 1 - DNA, 3220 bp, Homo sapiens
gaaggaccacagctcctcccgtgcatccactcggcctgggaggttctggattttggctgtcgagggagtttgcctgcctctccagagaaagatggtcatgaggcccctgtggagtctgcttctctgggaagccctacttcccattacagttactggtgcccaagtgctgagcaaagtcgggggctcggtgctgctggtggcagcgcgtccccctggcttccaagtccgtgaggctatctggcgatctctctggccttcagaagagctcctggccacgtttttccgaggctccctggagactctgtaccattcccgcttcctgggccgagcccagctacacagcaacctcagcctggagctcgggccgctggagtctggagacagcggcaacttctccgtgttgatggtggacacaaggggccagccctggacccagaccctccagctcaaggtgtacgatgcagtgcccaggcccgtggtacaagtgttcattgctgtagaaagggatgctcagccctccaagacctgccaggttttcttgtcctgttgggcccccaacatcagcgaaataacctatagctggcgacgggagacaaccatggactttggtatggaaccacacagcctcttcacagacggacaggtgctgagcatttccctgggaccaggagacagagatgtggcctattcctgcattgtctccaaccctgtcagctgggacttggccacagtcacgccctgggatagctgtcatcatgaggcagcaccagggaaggcctcctacaaagatgtgctgctggtggtggtgcctgtctcgctgctcctgatgctggttactctcttctctgcctggcactggtgcccctgctcagggaaaaagaaaaaggatgtccatgctgacagagtgggtccagagacagagaacccccttgtgcaggatctgccataaaggacaatatgaactgatgcctggactatcagtaaccccactgcacaggcacacgatgctctgggacataactggtgcctggaaatcaccatggtcctcatatctcccatgggaatcctgtcctgcctcgaaggagcagcctgggcagccatcacaccacgaggacaggaagcaccagcacgtttcacacctcccccttccctctcccatcttctcatatcctggctcttctctgggcaagatgagccaagcagaacattccatccaggacactggaagttctccaggatccagatccatggggacattaatagtccaaggcattccctcccccaccactattcataaagtattaaccaactggcaccaaggaattgcctccagcctgagtcctaggctctaaaagatattacatatttgaactaatagaggaactctgagtcacccatgccagcatcagcttcagccccagaccctgcagtttgagatctgatgcttcctgagggccaaggcattgctgtaagaaaaggtctagaaataggtgaaagtgagaggtgggggacaggggtttctctttctggcctaaggactttcaggtaatcagagttcatgggccctcaaaggtaaattgcagttgtagacaccgaggatggttgacaacccatggttgagatgggcaccgttttgcaggaaacaccatattaatagacatcctcaccatctccatccgctctcacgcctcctgcaggatctgggagtgagggtggagagtctttcctcacgctccagcacagtggccaggaaaagaaatactgaatttgccccagccaacaggacgttcttgcacaacttcaagaaaagcagctcagctcaggatgagtcttcctgcctgaaactgagagagtgaagaaccataaaacgctatgcagaaggaacattatggagagaaagggtactgaggcactctagaatctgccacattcattttcaaatgcaaatgcagaagacttaccttagttcaaggggaggggacaaagaccccacagcccaacagcaggactgtagaggtcactctgactccatcaaactttttattgtggccatcttaggaaaatacattctgcccctgaatgattctgtctagaaaagctctggagtattgatcactactggaaaaacacttaaggagctaaacttaccttcggggattattagctgataaggttcacagtttctctcacccaggtgtaactggattttttctggggcctcaatccagtcttgataacagcgaggaaagaggtattgaagaaacaggggtgggtttgaagtactattttccccagggtggcttcaatctccccacctaggatgtcagccctgtccaaggaccttccctcttctcccccagttccctgggcaatcacttcaccttggacaaaggatcagcacagctggcctccagatccacatcaccactcttccactcgattgttcccagatcctccctgcctggcctgctcagaggttccctgttggtaacctggctttatcaaattctcatccctttcccacacccacttctctcctatcaccttcccccaagattacctgaacagggtccatggccactcaacctgtcagcttgcaccatccccacctgccacctacagtcaggccacatgcctggtcactgaatcatgcaaaactggcctcagtccctaaaaatgatgtggaaaggaaagcccaggatctgacaatgagccctggtggatttgtggggaaaaaatacacagcactccccacctttctttcgttcatctccagggccccacctcagatcaaagcagctctggatgagatgggacctgcagctctccctccacaaggtgactcttagcaacctcatttcgacagtggtttgtagcgtggtgcaccagggccttgttgaacagatccacactgctctaataaagttcccatccttaatgactcacttgtcaactagtggactaattaaccctccaccaaaaaaacacaaagtgcttctgtgagaccaattttgtgctaatgagcattgagactgatgctttgtaagtcacaccacaacaaatattgattgagggcgctgcatgtgctgggtacatttcttggcacttgggaatcagtagtcaagcgaaacccttgcctttgagagtttatggtctggataatataaataaacaagtaagcataaaaaaaaaaaaaaaaaaaaaaaaaaaaaaaaaaaaa

SEQ ID NO: 2 - protein, 285 aa, Homo sapiens
MVMRPLWSLLLWEALLPITVTGAQVLSKVGGSVLLVAARPPGFQVREAIWRSLWPSEELLATFFRGSLETLYHSRFLGRAQLHSNLSLELGPLESGDSGNFSVLMVDTRGQPWTQTLQLKVYDAVPRPVVQVFIAVERDAQPSKTCQVFLSCWAPNISEITYSWRRETTMDFGMEPHSLFTDGQVLSISLGPGDRDVAYSCIVSNPVSWDLATVTPWDSCHHEAAPGKASYKDVLLVVVPVSLLLMLVTLFSAWHWCPCSGKKKKDVHADRVGPETENPLVQDLP

SEQ ID NO: 3 - protein, 335 aa, Homo sapiens
MDPKGLLSLTFVLFLSLAFGASYGTGGRMMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFSPWPGCRTDPSETKPWAVYAGLLGGVIMILIMVVILQLRRRGKTNHYQTTVEKKSLTIYAQVQKPGPLQKKLDSFPAQDPCTTIYVAATEPVPESVQETNSITVYASVTLPES

SEQ ID NO: 4 - DNA, 733 bp, Homo sapiens
gggatccggagcccaaatcttctgacaaaactcacacatgcccaccgtgcccagcacctgaattcgagggtgcaccgtcagtcttcctcttccccccaaaacccaaggacaccctcatgatctcccggactcctgaggtcacatgcgtggtggtggacgtaagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaacccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccgcgactctagaggat

SEQ ID NO: 5 - protein, 5 aa, Homo sapiens
  SITE (3): Xaa equals any amino acid
WSXWS

SEQ ID NO: 6 - DNA, 86 bp, Homo sapiens
gcgcctcgagatttccccgaaatctagatttccccgaaatgatttccccgaaatgatttccccgaaatatctgccatctcaattag

SEQ ID NO: 7 - DNA, 27 bp, Homo sapiens
gcggcaagctttttgcaaagcctaggc

SEQ ID NO: 8 - DNA, 271 bp, Homo sapiens
ctcgagatttccccgaaatctagatttccccgaaatgatttccccgaaatgatttccccgaaatatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 9 - DNA, 32 bp, Homo sapiens
gcgctcgagggatgacagcgatagaaccccgg

SEQ ID NO: 10 - DNA, 31 bp, Homo sapiens
gcgaagcttcgcgactccccggatccgcctc

SEQ ID NO: 11 - DNA, 12 bp, Homo sapiens
ggggactttccc

SEQ ID NO: 12 - DNA, 73 bp, Homo sapiens
gcggcctcgaggggactttcccggggactttccggggactttccgggactttccatcctgccatctcaattag

SEQ ID NO: 13 - DNA, 256 bp, Homo sapiens
ctcgaggggactttcccggggactttccggggactttccgggactttccatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

(19) United States
(12) Patent Application Publication
(10) Pub. No.: US 2007/0042416 A1
(43) Pub. Date: Feb. 22, 2007

(54) Dendritic Enriched Secreted Lymphocyte Activation Molecule

(75) Inventors: Paul Young, Gaithersburg, MD (US); Steven M. Ruben, Brookeville, MD (US)

Correspondence Address: HUMAN GENOME SCIENCES INC.;INTELLECTUAL PROPERTY DEPT., 14200 SHADY GROVE ROAD, ROCKVILLE, MD, 20850, US

(73) Assignee: Human Genome Sciences, Inc., Rockville, MD (US)

(21) Appl. No.: 11/537,686

(22) Filed: Oct. 2, 2006

Related U.S. Application Data
(63) Continuation of application No. 10/445,888, filed on May 28, 2003.
Application No. 10/445,888 is a continuation of application No. 09/244,110, filed on Feb. 4, 1999, now abandoned.
(60) Provisional application No. 60/073,962, filed on Feb. 6, 1998.
(60) Provisional application No. 60/078,572, filed on Mar. 19, 1998.

(51) Int. Cl. C12Q 1/68; G01N 33/53; C07H 21/04; C07K 14/47

(52) U.S. Cl. 435/6; 530/350; 435/7.1; 435/320.1; 435/325; 536/23.5

(57) ABSTRACT

The present invention relates to a novel human protein called Dendritic Enriched Secreted Lymphocyte Activation Molecule, and isolated polynucleotides encoding this protein. Also provided are vectors, host cells, antibodies, and recombinant methods for producing this human protein. The invention further relates to diagnostic and therapeutic methods useful for diagnosing and treating disorders related to this novel human protein.

This application is a continuation of U.S. patent application Ser. No. 10/445,888, filed May 28, 2003, which is a continuation of U.S. patent application Ser. No. 09/244,110, filed Feb. 4, 1999, which claims benefit under 35 U.S.C. § 119(e) of U.S. Provisional Application Ser. No. 60/073,962, filed Feb. 6, 1998, and Provisional Application Ser. No. 60/078,572, filed Mar. 19, 1998, each of which is hereby incorporated by reference herein in its entirety.

### FIELD OF THE INVENTION

The present invention relates to a novel human gene encoding a polypeptide which is a member of the Secreted Lymphocyte Activation Molecule (SLAM) family. More specifically, the present invention relates to a polynucleotide encoding a novel human polypeptide named Dendritic Enriched Secreted Lymphocyte Activation Molecule, or “D-SLAM.” This invention also relates to D-SLAM polypeptides, as well as vectors, host cells, antibodies directed to D-SLAM polypeptides, and the recombinant methods for producing the same. Also provided are diagnostic methods for detecting disorders related to the immune system, and therapeutic methods for treating such disorders. The invention further relates to screening methods for identifying agonists and antagonists of D-SLAM activity.

### BACKGROUND OF THE INVENTION

A member of the immunoglobulin gene superfamily, SLAM is rapidly induced after activation of naive T- and B-cells. (Cocks, B. G., “A Novel Receptor Involved in T-Cell Activation,” Nature 376:260-263 (1995); Aversa, G., “Engagement of the Signaling Lymphocytic Activation Molecule (SLAM) on Activated T Cells Results in Il-2-Independent, Cyclosporin A-Sensitive T Cell Proliferation and IFN-γ Production,” J. Immun. 4036-4044 (1997).) A multifunctional 70 kDa glycoprotein, SLAM causes proliferation and differentiation of immune cells. (Punnonen, J., “Soluble and Membrane-bound Forms of Signaling Lymphocytic Activation Molecule (SLAM) Induce Proliferation and Ig Synthesis by Activated Human B Lymphocytes,” J. Exp. Med. 185:993-1004 (1997).) To elicit an immune response, both a secreted form of SLAM, as well as a membrane bounded SLAM, are thought to interact.

It is also known that dendritic cells (DC) are the principal antigen presenting cells involved in primary immune responses; their major function is to obtain antigen in tissues, migrate to lymphoid organs, and activate T cells. (Mohamadzadeh, M. et al., J. Immunol. 156: 3102-3106 (1996).) In fact, DC are usually the first immune cells to arrive at sites of inflammation on mucous membranes. (See, e.g., Weissman, D. et al., J. Immunol. 155:4111-4117 (1995).) There is a constant need to identify new polypeptide factors which may mediate interactions between DC and T cells, leading to the activation and/or proliferation of immune cells. To date, however, SLAM molecules have not been identified on DC cells.

Thus, there is a need for polypeptides that affect the proliferation, activation, survival, and/or differentiation of immune cells, such as T- and B-cells, since disturbances of such regulation may be involved in disorders relating to immune system. Therefore, there is a need for identification and characterization of such human polypeptides which can play a role in detecting, preventing, ameliorating or correcting such disorders.

### SUMMARY OF THE INVENTION

The present invention relates to a novel polynucleotide and the encoded polypeptide of D-SLAM. Moreover, the present invention relates to vectors, host cells, antibodies, and recombinant methods for producing the polypeptides and polynucleotides. Also provided are diagnostic methods for detecting disorders relates to the polypeptides, and therapeutic methods for treating such disorders. The invention further relates to screening methods for identifying binding partners of D-SLAM.

### BRIEF DESCRIPTION OF THE DRAWINGS

FIGS. 1A-1D show the nucleotide sequence (SEQ ID NO:1) and the deduced amino acid sequence (SEQ ID NO:2) of D-SLAM. The predicted leader sequence located at about amino acids 1-22 is underlined.

FIG. 2 shows the regions of identity between the amino acid sequence of the D-SLAM protein and the translation product of the human SLAM (Accession No. gi/984969) (SEQ ID NO:3), determined by BLAST analysis. Identical amino acids between the two polypeptides are shaded, while conservative amino acid are boxed. By examining the regions of amino acids shaded and/or boxed, the skilled artisan can readily identify conserved domains between the two polypeptides. These conserved domains are preferred embodiments of the present invention.

FIG. 3 shows an analysis of the D-SLAM amino acid sequence. Alpha, beta, turn and coil regions; hydrophilicity and hydrophobicity; amphipathic regions; flexible regions; antigenic index and surface probability are shown, and all were generated using the default settings. In the “Antigenic Index or Jameson-Wolf” graph, the positive peaks indicate locations of the highly antigenic regions of the D-SLAM protein, i.e., regions from which epitope-bearing peptides of the invention can be obtained. The domains defined by these graphs are contemplated by the present invention. Tabular representation of the data summarized graphically in FIG. 3 can be found in Tables 1A-1I.

### DETAILED DESCRIPTION

Definitions

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide.

In the present invention, a “secreted” D-SLAM protein refers to a protein capable of being directed to the ER, secretory vesicles, or the extracellular space as a result of a signal sequence, as well as a D-SLAM protein released into the extracellular space without necessarily containing a signal sequence. If the D-SLAM secreted protein is released into the extracellular space, the D-SLAM secreted protein can undergo extracellular processing to produce a “mature” D-SLAM protein. Release into the extracellular space can occur by many mechanisms, including exocytosis and proteolytic cleavage.

As used herein, a D-SLAM “polynucleotide” refers to a molecule having a nucleic acid sequence contained in SEQ ID NO:1 or the cDNA contained within the clone deposited with the ATCC™. For example, the D-SLAM polynucleotide can contain the nucleotide sequence of the full length cDNA sequence, including the 5′ and 3′ untranslated sequences, the coding region, with or without the signal sequence, the secreted protein coding region, as well as fragments, epitopes, domains, and variants of the nucleic acid sequence. Moreover, as used herein, a D-SLAM “polypeptide” refers to a molecule having the translated amino acid sequence generated from the polynucleotide as broadly defined.

In specific embodiments, the polynucleotides of the invention are less than 300 kb, 200 kb, 100 kb, 50 kb, 15 kb, 10 kb, or 7.5 kb in length. In a further embodiment, polynucleotides of the invention comprise at least 15 contiguous nucleotides of D-SLAM coding sequence, but do not comprise all or a portion of any D-SLAM intron. In another embodiment, the nucleic acid comprising D-SLAM coding sequence does not contain coding sequences of a genomic flanking gene (i.e., 5′ or 3′ to the D-SLAM gene in the genome).

In the present invention, the full length D-SLAM sequence identified as SEQ ID NO:1 was generated by overlapping sequences of the deposited clone (contig analysis). A representative clone containing all or most of the sequence for SEQ ID NO:1 was deposited with the American Type Culture Collection (“ATCC™”) on Feb. 6, 1998, and was given the ATCC™ Deposit Number 209623. The ATCC™ is located at 10801 University Boulevard, Manassas, Va. 20110-2209, USA. The ATCC™ deposit was made pursuant to the terms of the Budapest Treaty on the international recognition of the deposit of microorganisms for purposes of patent procedure.

A D-SLAM “polynucleotide” also includes those polynucleotides capable of hybridizing, under stringent hybridization conditions, to sequences contained in SEQ ID NO:1, the complement thereof, or the cDNA within the deposited clone. “Stringent hybridization conditions” refers to an overnight incubation at 42 degree C. in a solution comprising 50% formamide, 5×SSC (750 mM NaCl, 75 mM sodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65 degree C.

Also contemplated are nucleic acid molecules that hybridize to the D-SLAM polynucleotides at moderately high stringency hybridization conditions. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 degree C. in a solution comprising 6×SSPE (20×SSPE=3M NaCl; 0.2M NaH₂PO₄; 0.02M EDTA, pH 7.4), 0.5% SDS, 30% formamide, 100 ug/ml salmon sperm blocking DNA; followed by washes at 50 degree C. with 1×SSPE, 0.1% SDS. In addition, to achieve even lower stringency, washes performed following stringent hybridization can be done at higher salt concentrations (e.g. 5×SSC).

Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Of course, a polynucleotide which hybridizes only to polyA+ sequences (such as any 3′ terminal polyA+ tract of a cDNA shown in the sequence listing), or to a complementary stretch of T (or U) residues, would not be included in the definition of “polynucleotide,” since such a polynucleotide would hybridize to any nucleic acid molecule containing a poly (A) stretch or the complement thereof (e.g., practically any double-stranded cDNA clone).

The D-SLAM polynucleotide can be composed of any polyribonucleotide or polydeoxribonucleotide, which may be unmodified RNA or DNA or modified RNA or DNA. For example, D-SLAM polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the D-SLAM polynucleotides can be composed of triple-stranded regions comprising RNA or DNA or both RNA and DNA. D-SLAM polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

D-SLAM polypeptides can be composed of amino acids joined to each other by peptide bonds or modified peptide bonds, i.e., peptide isosteres, and may contain amino acids other than the 20 gene-encoded amino acids. The D-SLAM polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the D-SLAM polypeptide, including the peptide backbone, the amino acid side-chains and the amino or carboxyl termini. It will be appreciated that the same type of modification may be present in the same or varying degrees at several sites in a given D-SLAM polypeptide. Also, a given D-SLAM polypeptide may contain many types of modifications. D-SLAM polypeptides may be branched, for example, as a result of ubiquitination, and they may be cyclic, with or without branching. Cyclic, branched, and branched cyclic D-SLAM polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

“SEQ ID NO:1” refers to a D-SLAM polynucleotide sequence while “SEQ ID NO:2” refers to a D-SLAM polypeptide sequence.

A D-SLAM polypeptide “having biological activity” refers to polypeptides exhibiting activity similar, but not necessarily identical to, an activity of a D-SLAM polypeptide, including mature forms, as measured in a particular biological assay, with or without dose dependency. In the case where dose dependency does exist, it need not be identical to that of the D-SLAM polypeptide, but rather substantially similar to the dose-dependence in a given activity as compared to the D-SLAM polypeptide (i.e., the candidate polypeptide will exhibit greater activity or not more than about 25-fold less and, preferably, not more than about tenfold less activity, and most preferably, not more than about three-fold less activity relative to the D-SLAM polypeptide.)

D-SLAM Polynucleotides and Polypeptides

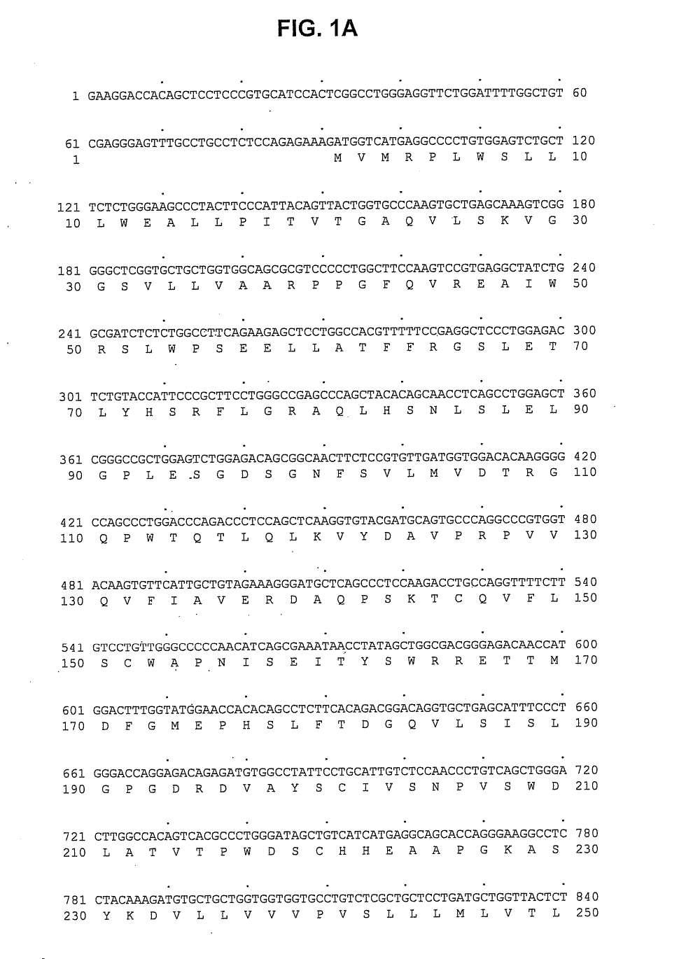

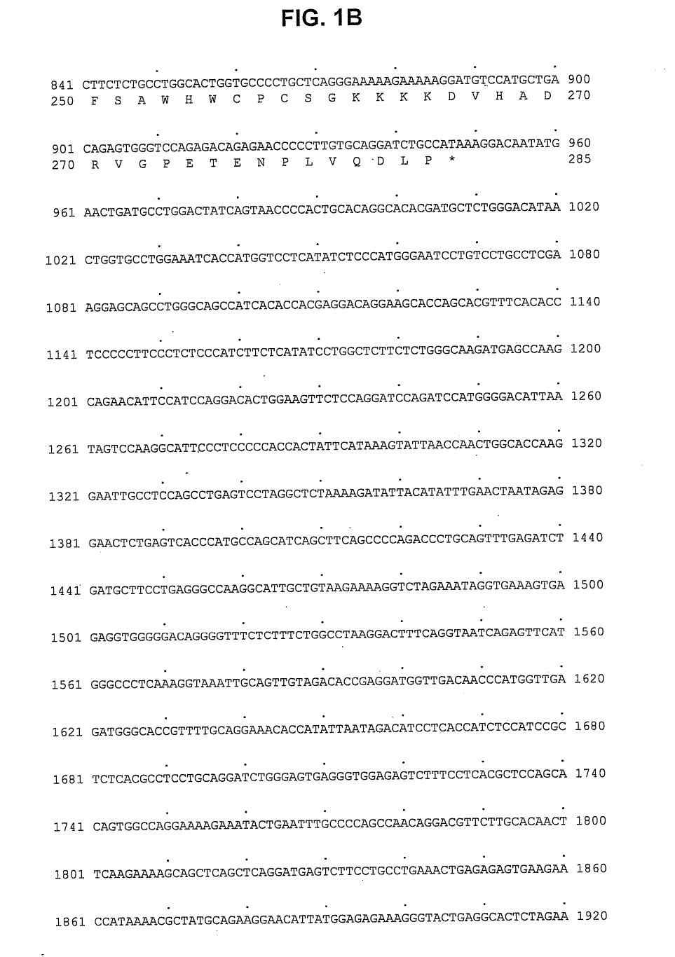

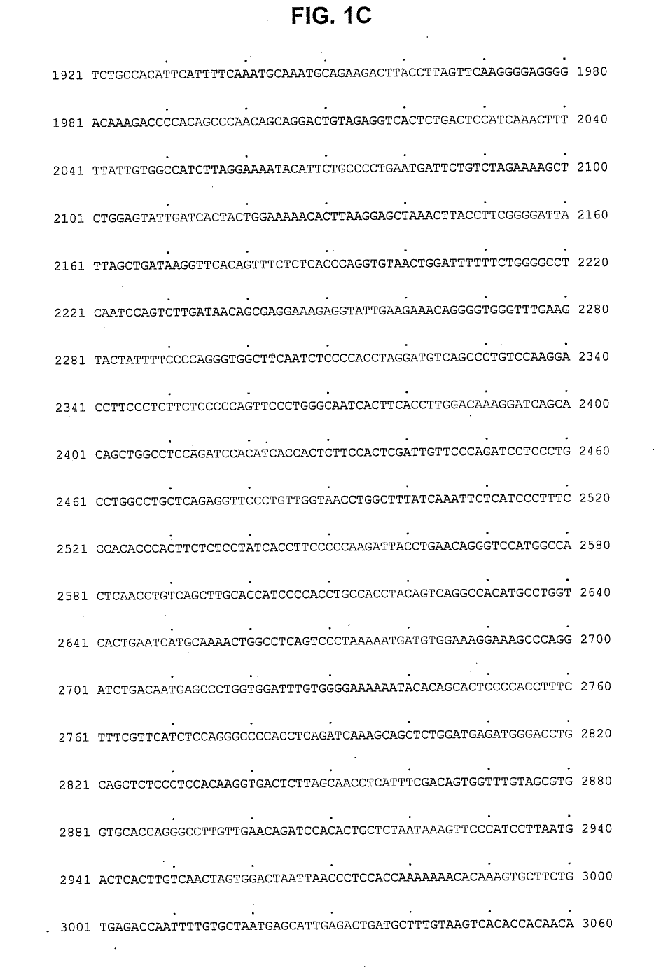

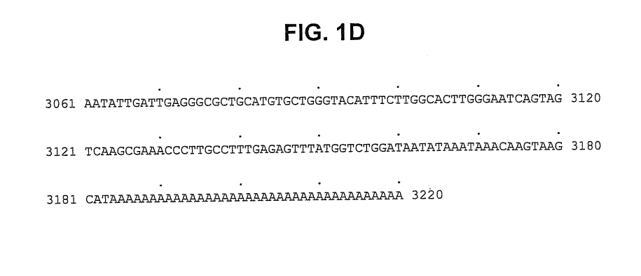

Clone HDPJO39 was isolated from a dendritic cell cDNA library. This clone contains the entire coding region identified as SEQ ID NO:2. The deposited clone contains a cDNA having a total of 3220 nucleotides, which encodes a predicted open reading frame of 285 amino acid residues. (See FIGS. 1A-1D.) The open reading frame begins at a N-terminal methionine located at nucleotide position 92, and ends at a stop codon at nucleotide position 947. The predicted molecular weight of the D-SLAM protein should be about 34.2 kDa.

Subsequent Northern analysis also showed D-SLAM expression in dendritic cells, T cell lymphoma, lymph node, spleen, thymus, small intestine, and uterus tissues, a pattern consistent with hematopoietic specific expression. Expression is highest in tissues involved in immune recognition, consistent with the enriched expression in dendritic cells and APC's. A single primary transcript of approximately 3.5-4.0 kb is observed, with a minor transcript of 7-9 kb that likely represents an unprocessed RNA precursor. The expression of the major 3.5-4 kb transcript is highest in lymph node, spleen, thymus, and, to a lesser degree, in small intestine. The highest expression of the 7-9 kb transcript is observed in the uterus.

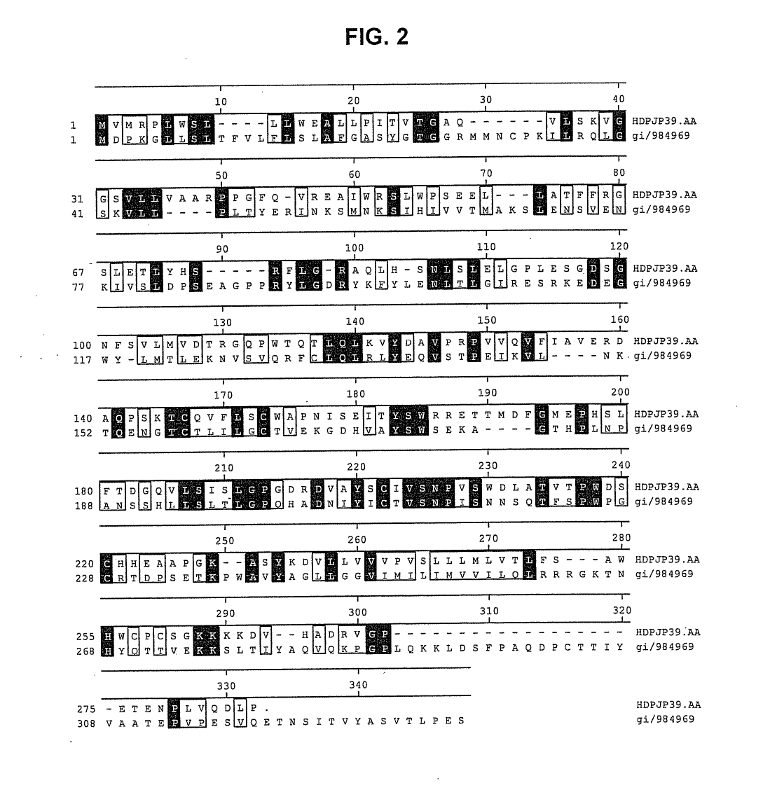

Using BLAST analysis, SEQ ID NO:2 was found to be homologous to members of the Secreted Lymphocyte Activation Molecule (SLAM) family. Particularly, SEQ ID NO:2 contains domains homologous to the translation product of the human mRNA for SLAM (Accession No. gi/984969) (FIG. 2) (SEQ ID NO:3), including the following conserved domains: (a) a predicted transmembrane domain located at about amino acids 233-255; (b) a predicted extracellular domain located at about amino acids 23-232; and (c) a predicted intracellular domain located at about amino acids 256-285. These polypeptide fragments of D-SLAM are specifically contemplated in the present invention. Because SLAM (Accession No. gi/984969) is thought to be important in the activation and proliferation of T- and B-cells, the homology between SLAM (Accession No. gi/984969) and D-SLAM suggests that D-SLAM may also be involved in the activation and proliferation of T- and B-cells.

Moreover, the encoded polypeptide has a predicted leader sequence located at about amino acids 1-22. (See FIGS. 1A-1D.) Also shown in FIGS. 1A-1D, the predicted secreted form of D-SLAM encompasses about amino acids 23-232. These polypeptide fragments of D-SLAM are specifically contemplated in the present invention.

The D-SLAM nucleotide sequence identified as SEQ ID NO:1 was assembled from partially homologous (“overlapping”) sequences obtained from the deposited clone, and in some cases, from additional related DNA clones. The overlapping sequences were assembled into a single contiguous sequence of high redundancy (usually three to five overlapping sequences at each nucleotide position), resulting in a final sequence identified as SEQ ID NO:1.

Therefore, SEQ ID NO:1 and the translated SEQ ID NO:2 are sufficiently accurate and otherwise suitable for a variety of uses well known in the art and described further below. For instance, SEQ ID NO:1 is useful for designing nucleic acid hybridization probes that will detect nucleic acid sequences contained in SEQ ID NO:1 or the cDNA contained in the deposited clone. These probes will also hybridize to nucleic acid molecules in biological samples, thereby enabling a variety of forensic and diagnostic methods of the invention. Similarly, polypeptides identified from SEQ ID NO:2 may be used to generate antibodies which bind specifically to D-SLAM.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

Accordingly, for those applications requiring precision in the nucleotide sequence or the amino acid sequence, the present invention provides not only the generated nucleotide sequence identified as SEQ ID NO:1 and the predicted translated amino acid sequence identified as SEQ ID NO:2, but also a sample of plasmid DNA containing a human cDNA of D-SLAM deposited with the ATCC™. The nucleotide sequence of the deposited D-SLAM clone can readily be determined by sequencing the deposited clone in accordance with known methods. The predicted D-SLAM amino acid sequence can then be verified from such deposits. Moreover, the amino acid sequence of the protein encoded by the deposited clone can also be directly determined by peptide sequencing or by expressing the protein in a suitable host cell containing the deposited human D-SLAM cDNA, collecting the protein, and determining its sequence.

The present invention also relates to the D-SLAM gene corresponding to SEQ ID NO:1, SEQ ID NO:2, or the deposited clone. The D-SLAM gene can be isolated in accordance with known methods using the sequence information disclosed herein. Such methods include preparing probes or primers from the disclosed sequence and identifying or amplifying the D-SLAM gene from appropriate sources of genomic material.

Also provided in the present invention are species homologs of D-SLAM. Species homologs may be isolated and identified by making suitable probes or primers from the sequences provided herein and screening a suitable nucleic acid source for the desired homologue.

The D-SLAM polypeptides can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

The D-SLAM polypeptides may be in the form of the secreted protein, including the mature form, or may be a part of a larger protein, such as a fusion protein (see below). It is often advantageous to include an additional amino acid sequence which contains secretory or leader sequences, pro-sequences, sequences which aid in purification, such as multiple histidine residues, or an additional sequence for stability during recombinant production.

D-SLAM polypeptides are preferably provided in an isolated form, and preferably are substantially purified. A recombinantly produced version of a D-SLAM polypeptide, including the secreted polypeptide, can be substantially purified by the one-step method described in Smith and Johnson, Gene 67:31-40 (1988). D-SLAM polypeptides also can be purified from natural or recombinant sources using antibodies of the invention raised against the D-SLAM protein in methods which are well known in the art.

Polynucleotide and Polypeptide Variants

“Variant” refers to a polynucleotide or polypeptide differing from the D-SLAM polynucleotide or polypeptide, but retaining essential properties thereof. Generally, variants are overall closely similar, and, in many regions, identical to the D-SLAM polynucleotide or polypeptide.

By a polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence encoding the D-SLAM polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence shown of SEQ ID NO:1, the ORF (open reading frame), or any fragment specified as described herein.

As a practical matter, whether any particular nucleic acid molecule or polypeptide is at least 90%, 95%, 96%, 97%, 98% or 99% identical to a nucleotide sequence of the presence invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245.) In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for. No other manual corrections are to made for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, (indels) or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequences shown in SEQ ID NO:2 or to the amino acid sequence encoded by deposited DNA clone can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for. No other manual corrections are to made for the purposes of the present invention.

The D-SLAM variants may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. D-SLAM polynucleotide variants can be produced for a variety of reasons, e.g., to optimize codon expression for a particular host (change codons in the human mRNA to those preferred by a bacterial host such as E. coli).

Naturally occurring D-SLAM variants are called “allelic variants,” and refer to one of several alternate forms of a gene occupying a given locus on a chromosome of an organism. (Genes II, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of the D-SLAM polypeptides. For instance, one or more amino acids can be deleted from the N-terminus or C-terminus of the secreted protein without substantial loss of biological function. The authors of Ron et al., J. Biol. Chem. 268: 2984-2988 (1993), reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus of this protein. (Dobeli et al., J. Biotechnology 7:199-216 (1988).)

Moreover, ample evidence demonstrates that variants often retain a biological activity similar to that of the naturally occurring protein. For example, Gayle and coworkers (J. Biol. Chem. 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type.

Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

Thus, the invention further includes D-SLAM polypeptide variants which show substantial biological activity. Such variants include deletions, insertions, inversions, repeats, and substitutions selected according to general rules known in the art so as have little effect on activity. For example, guidance concerning how to make phenotypically silent amino acid substitutions is provided in Bowie, J. U. et al., Science 247:1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. (Cunningham and Wells, Science 244:1081-1085 (1989).) The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Besides conservative amino acid substitution, variants of D-SLAM include (i) substitutions with one or more of the non-conserved amino acid residues, where the substituted amino acid residues may or may not be one encoded by the genetic code, or (ii) substitution with one or more of amino acid residues having a substituent group, or (iii) fusion of the mature polypeptide with another compound, such as a compound to increase the stability and/or solubility of the polypeptide (for example, polyethylene glycol), or (iv) fusion of the polypeptide with additional amino acids, such as an IgG Fc fusion region peptide, or leader or secretory sequence, or a sequence facilitating purification. Such variant polypeptides are deemed to be within the scope of those skilled in the art from the teachings herein.

For example, D-SLAM polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity. (Pinckard et al., Clin. Exp. Immunol. 2:331-340 (1967); Robbins et al., Diabetes 36: 838-845 (1987); Cleland et al., Crit. Rev. Therapeutic Drug Carrier Systems 10:307-377 (1993).)

A further embodiment of the invention relates to a polypeptide which comprises the amino acid sequence of a D-SLAM polypeptide having an amino acid sequence which contains at least one amino acid substitution, but not more than 50 amino acid substitutions, even more preferably, not more than 40 amino acid substitutions, still more preferably, not more than 30 amino acid substitutions, and still even more preferably, not more than 20 amino acid substitutions. Of course, in order of ever-increasing preference, it is highly preferable for a peptide or polypeptide to have an amino acid sequence which comprises the amino acid sequence of a D-SLAM polypeptide, which contains at least one, but not more than 10, 9, 8, 7, 6, 5, 4, 3, 2 or 1 amino acid substitutions. In specific embodiments, the number of additions, substitutions, and/or deletions in the amino acid sequence of FIGS. 1A-1D or fragments thereof (e.g., the mature form and/or other fragments described herein), is 1-5, 5-10, 5-25, 5-50, 10-50 or 50-150, conservative amino acid substitutions are preferable.

Polynucleotide and Polypeptide Fragments

In the present invention, a “polynucleotide fragment” refers to a short polynucleotide having a nucleic acid sequence contained in the deposited clone or shown in SEQ ID NO:1. The short nucleotide fragments are preferably at least about 15 nt, and more preferably at least about 20 nt, still more preferably at least about 30 nt, and even more preferably, at least about 40 nt in length. A fragment “at least 20 nt in length,” for example, is intended to include 20 or more contiguous bases from the cDNA sequence contained in the deposited clone or the nucleotide sequence shown in SEQ ID NO:1. These nucleotide fragments are useful as diagnostic probes and primers as discussed herein. Of course, larger fragments (e.g., 50, 150, 500, 600, 2000 nucleotides) are preferred.

Moreover, representative examples of D-SLAM polynucleotide fragments include, for example, fragments having a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, or 900 to the end of SEQ ID NO:1 or the cDNA contained in the deposited clone. In this context “about” includes the particularly recited ranges, larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein.

In the present invention, a “polypeptide fragment” refers to a short amino acid sequence contained in SEQ ID NO:2 or encoded by the cDNA contained in the deposited clone. Protein fragments may be “free-standing,” or comprised within a larger polypeptide of which the fragment forms a part or region, most preferably as a single continuous region. Representative examples of polypeptide fragments of the invention, include, for example, fragments from about amino acid number 1-20, 21-40, 41-60, 61-80, 81-100, 102-120, 121-140, 141-160, 161-180, 181-200, 201-220, 221-240, 241-260, 261-280, or 281 to the end of the coding region. Moreover, polypeptide fragments can be about 20, 30, 40, 50, 60, 70, 80, 90, 100, 110, 120, 130, 140, or 150 amino acids in length. In this context “about” includes the particularly recited ranges, larger or smaller by several (5, 4, 3, 2, or 1) amino acids, at either extreme or at both extremes.

Preferred polypeptide fragments include the secreted D-SLAM protein as well as the mature form. Further preferred polypeptide fragments include the secreted D-SLAM protein or the mature form having a continuous series of deleted residues from the amino or the carboxy terminus, or both. For example, any number of amino acids, ranging from 1-60, can be deleted from the amino terminus of either the secreted D-SLAM polypeptide or the mature form. Similarly, any number of amino acids, ranging from 1-30, can be deleted from the carboxy terminus of the secreted D-SLAM protein or mature form. Furthermore, any combination of the above amino and carboxy terminus deletions are preferred. Similarly, polynucleotide fragments encoding these D-SLAM polypeptide fragments are also preferred.

Particularly, N-terminal deletions of the D-SLAM polypeptide can be described by the general formula m−285, where m is an integer from 2 to 284, where m corresponds to the position of the amino acid residue identified in SEQ ID NO:2. More in particular, the invention provides polynucleotides encoding polypeptides comprising, or alternatively consisting of, the amino acid sequence of residues of: V-2 to P-285; M-3 to P-285; R-4 to P-285; P-5 to P-285; L-6 to P-285; W-7 to P-285; S-8 to P-285; L-9 to P-285; L-10 to P-285; L-11 to P-285; W-12 to P-285; E-13 to P-285; A-14 to P-285; L-15 to P-285; L-16 to P-285; P-17 to P-285; I-18 to P-285; T-19 to P-285; V-20 to P-285; T-21 to P-285; G-22 to P-285; A-23 to P-285; Q-24 to P-285; V-25 to P-285; L-26 to P-285; S-27 to P-285; K-28 to P-285; V-29 to P-285; G-30 to P-285; G-31 to P-285; S-32 to P-285; V-33 to P-285; L-34 to P-285; L-35 to P-285; V-36 to P-285; A-37 to P-285; A-38 to P-285; R-39 to P-285; P-40 to P-285; P-41 to P-285; G-42 to P-285; F-43 to P-285; Q-44 to P-285; V-45 to P-285; R-46 to P-285; E-47 to P-285; A-48 to P-285; I-49 to P-285; W-50 to P-285; R-51 to P-285; S-52 to P-285; L-53 to P-285; W-54 to P-285; P-55 to P-285; S-56 to P-285; E-57 to P-285; E-58 to P-285; L-59 to P-285; L-60 to P-285; A-61 to P-285; T-62 to P-285; F-63 to P-285; F-64 to P-285; R-65 to P-285; G-66 to P-285; S-67 to P-285; L-68 to P-285; E-69 to P-285; T-70 to P-285; L-71 to P-285; Y-72 to P-285; H-73 to P-285; S-74 to P-285; R-75 to P-285; F-76 to P-285; L-77 to P-285; G-78 to P-285; R-79 to P-285; A-80 to P-285; Q-81 to P-285; L-82 to P-285; H-83 to P-285; S-84 to P-285; N-85 to P-285; L-86 to P-285; S-87 to P-285; L-88 to P-285; E-89 to P-285; L-90 to P-285; G-91 to P-285; P-92 to P-285; L-93 to P-285; E-94 to P-285; S-95 to P-285; G-96 to P-285; D-97 to P-285; S-98 to P-285; G-99 to P-285; N-100 to P-285; F-101 to P-285; S-102 to P-285; V-103 to P-285; L-104 to P-285; M-105 to P-285; V-106 to P-285; D-107 to P-285; T-108 to P-285; R-109 to P-285; G-110 to P-285; Q-111 to P-285; P-112 to P-285; W-113 to P-285; T-114 to P-285; Q-115 to P-285; T-116 to P-285; L-117 to P-285; Q-118 to P-285; L-119 to P-285; K-120 to P-285; V-121 to P-285; Y-122 to P-285; D-123 to P-285; A-124 to P-285; V-125 to P-285; P-126 to P-285; R-127 to P-285; P-128 to P-285; V-129 to P-285; V-130 to P-285; Q-131 to P-285; V-132 to P-285; F-133 to P-285; I-134 to P-285; A-135 to P-285; V-136 to P-285; E-137 to P-285; R-138 to P-285; D-139 to P-285; A-140 to P-285; Q-141 to P-285; P-142 to P-285; S-143 to P-285; K-144 to P-285; T-145 to P-285; C-146 to P-285; Q-147 to P-285; V-148 to P-285; F-149 to P-285; L-150 to P-285; S-151 to P-285; C-152 to P-285; W-153 to P-285; A-154 to P-285; P-155 to P-285; N-156 to P-285; I-157 to P-285; S-158 to P-285; E-159 to P-285; I-160 to P-285; T-161 to P-285; Y-162 to P-285; S-163 to P-285; W-164 to P-285; R-165 to P-285; R-166 to P-285; E-167 to P-285; T-168 to P-285; T-169 to P-285; M-170 to P-285; D-171 to P-285; F-172 to P-285; G-173 to P-285; M-174 to P-285; E-175 to P-285; P-176 to P-285; H-177 to P-285; S-178 to P-285; L-179 to P-285; F-180 to P-285; T-181 to P-285; D-182 to P-285; G-183 to P-285; Q-184 to P-285; V-185 to P-285; L-186 to P-285; S-187 to P-285; I-188 to P-285; S-189 to P-285; L-190 to P-285; G-191 to P-285; P-192 to P-285; G-193 to P-285; D-194 to P-285; R-195 to P-285; D-196 to P-285; V-197 to P-285; A-198 to P-285; Y-199 to P-285; S-200 to P-285; C-201 to P-285; I-202 to P-285; V-203 to P-285; S-204 to P-285; N-205 to P-285; P-206 to P-285; V-207 to P-285; S-208 to P-285; W-209 to P-285; D-210 to P-285; L-211 to P-285; A-212 to P-285; T-213 to P-285; V-214 to P-285; T-215 to P-285; P-216 to P-285; W-217 to P-285; D-218 to P-285; S-219 to P-285; C-220 to P-285; H-221 to P-285; H-222 to P-285; E-223 to P-285; A-224 to P-285; A-225 to P-285; P-226 to P-285; G-227 to P-285; K-228 to P-285; A-229 to P-285; S-230 to P-285; Y-231 to P-285; K-232 to P-285; D-233 to P-285; V-234 to P-285; L-235 to P-285; L-236 to P-285; V-237 to P-285; V-238 to P-285; V-239 to P-285; P-240 to P-285; V-241 to P-285; S-242 to P-285; L-243 to P-285; L-244 to P-285; L-245 to P-285; M-246 to P-285; L-247 to P-285; V-248 to P-285; T-249 to P-285; L-250 to P-285; F-251 to P-285; S-252 to P-285; A-253 to P-285; W-254 to P-285; H-255 to P-285; W-256 to P-285; C-257 to P-285; P-258 to P-285; C-259 to P-285; S-260 to P-285; G-261 to P-285; K-262 to P-285; K-263 to P-285; K-264 to P-285; K-265 to P-285; D-266 to P-285; V-267 to P-285; H-268 to P-285; A-269 to P-285; D-270 to P-285; R-271 to P-285; V-272 to P-285; G-273 to P-285; P-274 to P-285; E-275 to P-285; T-276 to P-285; E-277 to P-285; N-278 to P-285; P-279 to P-285; L-280 to P-285; of SEQ ID NO:2. Polynucleotides encoding these polypeptides are also encompassed by the invention.

Moreover, C-terminal deletions of the D-SLAM polypeptide can also be described by the general formula 1−n, where n is an integer from 2 to 284, where n corresponds to the position of amino acid residue identified in SEQ ID NO:2. More in particular, the invention provides polynucleotides encoding polypeptides comprising, or alternatively consisting of, the amino acid sequence of residues of: M-1 to L-284; M-1 to D-283; M-1 to Q-282; M-1 to V-281; M-1 to L-280; M-1 to P-279; M-1 to N-278; M-1 to E-277; M-1 to T-276; M-1 to E-275; M-1 to P-274; M-1 to G-273; M-1 to V-272; M-1 to R-271; M-1 to D-270; M-1 to A-269; M-1 to H-268; M-1 to V-267; M-1 to D-266; M-1 to K-265; M-1 to K-264; M-1 to K-263; M-1 to K-262; M-1 to G-261; M-1 to S-260; M-1 to C-259; M-1 to P-258; M-1 to C-257; M-1 to W-256; M-1 to H-255; M-1 to W-254; M-1 to A-253; M-1 to S-252; M-1 to F-251; M-1 to L-250; M-1 to T-249; M-1 to V-248; M-1 to L-247; M-1 to M-246; M-1 to L-245; M-1 to L-244; M-1 to L-243; M-1 to S-242; M-1 to V-241; M-1 to P-240; M-1 to V-239; M-1 to V-238; M-1 to V-237; M-1 to L-236; M-1 to L-235; M-1 to V-234; M-1 to D-233; M-1 to K-232; M-1 to Y-231; M-1 to S-230; M-1 to A-229; M-1 to K-228; M-1 to G-227; M-1 to P-226; M-1 to A-225; M-1 to A-224; M-1 to E-223; M-1 to H-222; M-1 to H-221; M-1 to C-220; M-1 to S-219; M-1 to D-218; M-1 to W-217; M-1 to P-216; M-1 to T-215; M-1 to V-214; M-1 to T-213; M-1 to A-212; M-1 to L-211; M-1 to D-210; M-1 to W-209; M-1 to S-208; M-1 to V-207; M-1 to P-206; M-1 to N-205; M-1 to S-204; M-1 to V-203; M-1 to I-202; M-1 to C-201; M-1 to S-200; M-1 to Y-199; M-1 to A-198; M-1 to V-197; M-1 to D-196; M-1 to R-195; M-1 to D-194; M-1 to G-193; M-1 to P-192; M-1 to G-191; M-1 to L-190; M-1 to S-189; M-1 to I-188; M-1 to S-187; M-1 to L-186; M-1 to V-185; M-1 to Q-184; M-1 to G-183; M-1 to D-182; M-1 to T-181; M-1 to F-180; M-1 to L-179; M-1 to S-178; M-1 to H-177; M-1 to P-176; M-1 to E-175; M-1 to M-174; M-1 to G-173; M-1 to F-172; M-1 to D-171; M-1 to M-170; M-1 to T-169; M-1 to T-168; M-1 to E-167; M-1 to R-166; M-1 to R-165; M-1 to W-164; M-1 to S-163; M-1 to Y-162; M-1 to T-161; M-1 to I-160; M-1 to E-159; M-1 to S-158; M-1 to I-157; M-1 to N-156; M-1 to P-155; M-1 to A-154; M-1 to W-153; M-1 to C-152; M-1 to S-151; M-1 to L-150; M-1 to F-149; M-1 to V-148; M-1 to Q-147; M-1 to C-146; M-1 to T-145; M-1 to K-144; M-1 to S-143; M-1 to P-142; M-1 to Q-141; M-1 to A-140; M-1 to D-139; M-1 to R-138; M-1 to E-137; M-1 to V-136; M-1 to A-135; M-1 to I-134; M-1 to F-133; M-1 to V-132; M-1 to Q-131; M-1 to V-130; M-1 to V-129; M-1 to P-128; M-1 to R-127; M-1 to P-126; M-1 to V-125; M-1 to A-124; M-1 to D-123; M-1 to Y-122; M-1 to V-121; M-1 to K-120; M-1 to L-119; M-1 to Q-118; M-1 to L-117; M-1 to T-116; M-1 to Q-115; M-1 to T-114; M-1 to W-113; M-1 to P-112; M-1 to Q-111; M-1 to G-110; M-1 to R-109; M-1 to T-108; M-1 to D-107; M-1 to V-106; M-1 to M-105; M-1 to L-104; M-1 to V-103; M-1 to S-102; M-1 to F-101; M-1 to N-100; M-1 to G-99; M-1 to S-98; M-1 to D-97; M-1 to G-96; M-1 to S-95; M-1 to E-94; M-1 to L-93; M-1 to P-92; M-1 to G-91; M-1 to L-90; M-1 to E-89; M-1 to L-88; M-1 to S-87; M-1 to L-86; M-1 to N-85; M-1 to S-84; M-1 to H-83; M-1 to L-82; M-1 to Q-81; M-1 to A-80; M-1 to R-79; M-1 to G-78; M-1 to L-77; M-1 to F-76; M-1 to R-75; M-1 to S-74; M-1 to H-73; M-1 to Y-72; M-1 to L-71; M-1 to T-70; M-1 to E-69; M-1 to L-68; M-1 to S-67; M-1 to G-66; M-1 to R-65; M-1 to F-64; M-1 to F-63; M-1 to T-62; M-1 to A-61; M-1 to L-60; M-1 to L-59; M-1 to E-58; M-1 to E-57; M-1 to S-56; M-1 to P-55; M-1 to W-54; M-1 to L-53; M-1 to S-52; M-1 to R-51; M-1 to W-50; M-1 to I-49; M-1 to A-48; M-1 to E-47; M-1 to R-46; M-1 to V-45; M-1 to Q-44; M-1 to F-43; M-1 to G-42; M-1 to P-41; M-1 to P-40; M-1 to R-39; M-1 to A-38; M-1 to A-37; M-1 to V-36; M-1 to L-35; M-1 to L-34; M-1 to V-33; M-1 to S-32; M-1 to G-31; M-1 to G-30; M-1 to V-29; M-1 to K-28; M-1 to S-27; M-1 to L-26; M-1 to V-25; M-1 to Q-24; M-1 to A-23; M-1 to G-22; M-1 to T-21; M-1 to V-20; M-1 to T-19; M-1 to I-18; M-1 to P-17; M-1 to L-16; M-1 to L-15; M-1 to A-14; M-1 to E-13; M-1 to W-12; M-1 to L-11; M-1 to L-10; M-1 to L-9; M-1 to S-8; M-1 to W-7; of SEQ ID NO:2. Polynucleotides encoding these polypeptides are also encompassed by the invention.

In addition, any of the above listed N- or C-terminal deletions can be combined to produce a N- and C-terminal deleted D-SLAM polypeptide. The invention also provides polypeptides having one or more amino acids deleted from both the amino and the carboxyl termini, which may be described generally as having residues m-n of SEQ ID NO:2, where n and m are integers as described above. Polynucleotides encoding these polypeptides are also encompassed by the invention.

Moreover, preferred N- and C-terminal deletion mutants comprise, or in the alternative consists of, the predicted secreted form of D-SLAM. Preferred secreted forms of the D-SLAM include polypeptides comprising the amino acid sequence of residues: M-1 to K-232; V-2 to K-232; M-3 to K-232; R-4 to K-232; P-5 to K-232; L-6 to K-232; W-7 to K-232; S-8 to K-232; L-9 to K-232; L-10 to K-232; L-11 to K-232; W-12 to K-232; E-13 to K-232; A-14 to K-232; L-15 to K-232; L-16 to K-232; P-17 to K-232; I-18 to K-232; T-19 to K-232; V-20 to K-232; T-21 to K-232; G-22 to K-232; A-23 to K-232; Q-24 to K-232; V-25 to K-232; L-26 to K-232; S-27 to K-232; K-28 to K-232; V-29 to K-232; G-30 to K-232; G-31 to K-232; S-32 to K-232; V-33 to K-232; L-34 to K-232; L-35 to K-232; V-36 to K-232; A-37 to K-232; A-38 to K-232; R-39 to K-232; P-40 to K-232; P-41 to K-232; G-42 to K-232; F-43 to K-232; Q-44 to K-232; V-45 to K-232; R-46 to K-232; E-47 to K-232; A-48 to K-232; I-49 to K-232; W-50 to K-232; R-51 to K-232; S-52 to K-232; L-53 to K-232; W-54 to K-232; P-55 to K-232; S-56 to K-232; E-57 to K-232; E-58 to K-232; L-59 to K-232; L-60 to K-232; A-61 to K-232; T-62 to K-232; F-63 to K-232; F-64 to K-232; R-65 to K-232; G-66 to K-232; S-67 to K-232; L-68 to K-232; E-69 to K-232; T-70 to K-232; L-71 to K-232; Y-72 to K-232; H-73 to K-232; S-74 to K-232; R-75 to K-232; F-76 to K-232; L-77 to K-232; G-78 to K-232; R-79 to K-232; A-80 to K-232; Q-81 to K-232; L-82 to K-232; H-83 to K-232; S-84 to K-232; N-85 to K-232; L-86 to K-232; S-87 to K-232; L-88 to K-232; E-89 to K-232; L-90 to K-232; G-91 to K-232; P-92 to K-232; L-93 to K-232; E-94 to K-232; S-95 to K-232; G-96 to K-232; D-97 to K-232; S-98 to K-232; G-99 to K-232; N-100 to K-232; F-101 to K-232; S-102 to K-232; V-103 to K-232; L-104 to K-232; M-105 to K-232; V-106 to K-232; D-107 to K-232; T-108 to K-232; R-109 to K-232; G-110 to K-232; Q-111 to K-232; P-112 to K-232; W-113 to K-232; T-114 to K-232; Q-115 to K-232; T-116 to K-232; L-117 to K-232; Q-118 to K-232; L-119 to K-232; K-120 to K-232; V-121 to K-232; Y-122 to K-232; D-123 to K-232; A-124 to K-232; V-125 to K-232; P-126 to K-232; R-127 to K-232; P-128 to K-232; V-129 to K-232; V-130 to K-232; Q-131 to K-232; V-132 to K-232; F-133 to K-232; I-134 to K-232; A-135 to K-232; V-136 to K-232; E-137 to K-232; R-138 to K-232; D-139 to K-232; A-140 to K-232; Q-141 to K-232; P-142 to K-232; S-143 to K-232; K-144 to K-232; T-145 to K-232; C-146 to K-232; Q-147 to K-232; V-148 to K-232; F-149 to K-232; L-150 to K-232; S-151 to K-232; C-152 to K-232; W-153 to K-232; A-154 to K-232; P-155 to K-232; N-156 to K-232; I-157 to K-232; S-158 to K-232; E-159 to K-232; I-160 to K-232; T-161 to K-232; Y-162 to K-232; S-163 to K-232; W-164 to K-232; R-165 to K-232; R-166 to K-232; E-167 to K-232; T-168 to K-232; T-169 to K-232; M-170 to K-232; D-171 to K-232; F-172 to K-232; G-173 to K-232; M-174 to K-232; E-175 to K-232; P-176 to K-232; H-177 to K-232; S-178 to K-232; L-179 to K-232; F-180 to K-232; T-181 to K-232; D-182 to K-232; G-183 to K-232; Q-184 to K-232; V-185 to K-232; L-186 to K-232; S-187 to K-232; I-188 to K-232; S-189 to K-232; L-190 to K-232; G-191 to K-232; P-192 to K-232; G-193 to K-232; D-194 to K-232; R-195 to K-232; D-196 to K-232; V-197 to K-232; A-198 to K-232; Y-199 to K-232; S-200 to K-232; C-201 to K-232; I-202 to K-232; V-203 to K-232; S-204 to K-232; N-205 to K-232; P-206 to K-232; V-207 to K-232; S-208 to K-232; W-209 to K-232; D-210 to K-232; L-211 to K-232; A-212 to K-232; T-213 to K-232; V-214 to K-232; T-215 to K-232; P-216 to K-232; W-217 to K-232; D-218 to K-232; S-219 to K-232; C-220 to K-232; H-221 to K-232; H-222 to K-232; E-223 to K-232; A-224 to K-232; A-225 to K-232; P-226 to K-232; G-227 to K-232; of SEQ ID NO:2. Polynucleotides encoding these polypeptides are also encompassed by the invention.

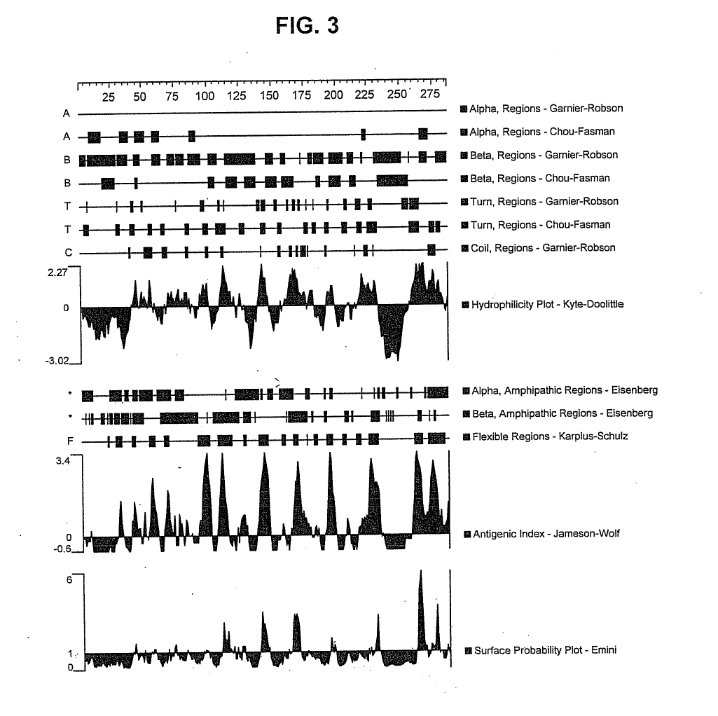

Also preferred are D-SLAM polypeptide and polynucleotide fragments characterized by structural or functional domains. Preferred embodiments of the invention include fragments that comprise alpha-helix and alpha-helix forming regions (“alpha-regions”), beta-sheet and beta-sheet-forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, flexible regions, surface-forming regions, substrate binding region, and high antigenic index regions. As set out in the Figures, such preferred regions include Garnier-Robson alpha-regions, beta-regions, turn-regions, and coil-regions, Chou-Fasman alpha-regions, beta-regions, and turn-regions, Kyte-Doolittle hydrophilic regions and hydrophobic regions, Eisenberg alpha and beta amphipathic regions, Karplus-Schulz flexible regions, Emini surface-forming regions, and Jameson-Wolf high antigenic index regions. Polypeptide fragments of SEQ ID NO:2 falling within conserved domains are specifically contemplated by the present invention. (See FIG. 3.) Moreover, polynucleotide fragments encoding these domains are also contemplated.

Other preferred fragments are biologically active D-SLAM fragments. Biologically active fragments are those exhibiting activity similar, but not necessarily identical, to an activity of the D-SLAM polypeptide. The biological activity of the fragments may include an improved desired activity, or a decreased undesirable activity.

However, many polynucleotide sequences, such as EST sequences, are publicly available and accessible through sequence databases. Some of these sequences are related to SEQ ID NO:1 and may have been publicly available prior to conception of the present invention. Preferably, such related polynucleotides are specifically excluded from the scope of the present invention. For example, the following ESTs are preferably excluded from the present invention: AA917335; AI094818, AI298413; N62522; AA627522; R11635; AA320408; AA379112; R09841; Z20320; N79421; D45800; T98959; AA217290; N30197; AA286132; and AA633983 (hereby incorporated by reference in their entirety.) However, to list every related sequence would be cumbersome. Accordingly, preferably excluded from the present invention are one or more polynucleotides comprising a nucleotide sequence described by the general formula of a−b, where a is any integer between 1 to 3206 of SEQ ID NO:1, b is an integer of 15 to 3220, where both a and b correspond to the positions of nucleotide residues shown in SEQ ID NO:1, and where the b is greater than or equal to a+14.

Epitopes & Antibodies

In the present invention, “epitopes” refer to D-SLAM polypeptide fragments having antigenic or immunogenic activity in an animal, especially in a human. A preferred embodiment of the present invention relates to a D-SLAM polypeptide fragment comprising an epitope, as well as the polynucleotide encoding this fragment. A region of a protein molecule to which an antibody can bind is defined as an “antigenic epitope.” In contrast, an “immunogenic epitope” is defined as a part of a protein that elicits an antibody response. (See, for instance, Geysen et al., Proc. Natl. Acad. Sci. USA 81:3998-4002 (1983).)

Fragments which function as epitopes may be produced by any conventional means. (See, e.g., Houghten, R. A., Proc. Natl. Acad. Sci. USA 82:5131-5135 (1985) further described in U.S. Pat. No. 4,631,211.)

In the present invention, antigenic epitopes preferably contain a sequence of at least seven, more preferably at least nine, and most preferably between about 15 to about 30 amino acids. Antigenic epitopes are useful to raise antibodies, including monoclonal antibodies, that specifically bind the epitope. (See, for instance, Wilson et al., Cell 37:767-778 (1984); Sutcliffe, J. G. et al., Science 219:660-666 (1983).)

Similarly, immunogenic epitopes can be used to induce antibodies according to methods well known in the art. (See, for instance, Sutcliffe et al., supra; Wilson et al., supra; Chow, M. et al., Proc. Natl. Acad. Sci. USA 82:910-914; and Bittle, F. J. et al., J. Gen. Virol. 66:2347-2354 (1985).) A preferred immunogenic epitope includes the secreted protein. The immunogenic epitopes may be presented together with a carrier protein, such as an albumin, to an animal system (such as rabbit or mouse) or, if it is long enough (at least about 25 amino acids), without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting.)

Using DNAstar analysis, SEQ ID NO:2 was found antigenic at amino acids: 29-32, 39-45, 48-50, 52-59, 64-72, 76-78, 91-101, 106-114, 121-128, 136-146, 162-178, 190-198, 216-233, and 257-285. Thus, these regions could be used as epitopes to produce antibodies against the protein encoded by HDPJO39.

As used herein, the term “antibody” (Ab) or “monoclonal antibody” (Mab) is meant to include intact molecules as well as antibody fragments (such as, for example, Fab and F(ab′)2 fragments) which are capable of specifically binding to protein. Fab and F(ab′)2 fragments lack the Fc fragment of intact antibody, clear more rapidly from the circulation, and may have less non-specific tissue binding than an intact antibody. (Wahl et al., J. Nucl. Med. 24:316-325 (1983).) Thus, these fragments are preferred, as well as the products of a FAB or other immunoglobulin expression library. Moreover, antibodies of the present invention include chimeric, single chain, and humanized antibodies.

Fusion Proteins

Any D-SLAM polypeptide can be used to generate fusion proteins. For example, the D-SLAM polypeptide, when fused to a second protein, can be used as an antigenic tag. Antibodies raised against the D-SLAM polypeptide can be used to indirectly detect the second protein by binding to the D-SLAM. Moreover, because secreted proteins target cellular locations based on trafficking signals, the D-SLAM polypeptides can be used as a targeting molecule once fused to other proteins.

Examples of domains that can be fused to D-SLAM polypeptides include not only heterologous signal sequences, but also other heterologous functional regions. The fusion does not necessarily need to be direct, but may occur through linker sequences.

Moreover, fusion proteins may also be engineered to improve characteristics of the D-SLAM polypeptide. For instance, a region of additional amino acids, particularly charged amino acids, may be added to the N-terminus of the D-SLAM polypeptide to improve stability and persistence during purification from the host cell or subsequent handling and storage. Also, peptide moieties may be added to the D-SLAM polypeptide to facilitate purification. Such regions may be removed prior to final preparation of the D-SLAM polypeptide. The addition of peptide moieties to facilitate handling of polypeptides are familiar and routine techniques in the art.

Moreover, D-SLAM polypeptides, including fragments, and specifically epitopes, can be combined with parts of the constant domain of immunoglobulins (IgG), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins. (EP A 394,827; Traunecker et al., Nature 331:84-86 (1988).) Fusion proteins having disulfide-linked dimeric structures (due to the IgG) can also be more efficient in binding and neutralizing other molecules, than the monomeric secreted protein or protein fragment alone. (Fountoulakis et al., J. Biochem. 270:3958-3964 (1995).)

Similarly, EP-A-O 464 533 (Canadian counterpart 2045869) discloses fusion proteins comprising various portions of constant region of immunoglobulin molecules together with another human protein or part thereof. In many cases, the Fc part in a fusion protein is beneficial in therapy and diagnosis, and thus can result in, for example, improved pharmacokinetic properties. (EP-A 0232 262.) Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5. (See, D. Bennett et al., J. Molecular Recognition 8:52-58 (1995); K. Johanson et al., J. Biol. Chem. 270:9459-9471 (1995).)

Moreover, the D-SLAM polypeptides can be fused to marker sequences, such as a peptide which facilitates purification of D-SLAM. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Another peptide tag useful for purification, the “HA” tag, corresponds to an epitope derived from the influenza hemagglutinin protein. (Wilson et al., Cell 37:767 (1984).)

Thus, any of these above fusions can be engineered using the D-SLAM polynucleotides or the polypeptides.

Vectors, Host Cells, and Protein Production

The present invention also relates to vectors containing the D-SLAM polynucleotide, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

D-SLAM polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The D-SLAM polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that D-SLAM polypeptides may in fact be expressed by a host cell lacking a recombinant vector.

D-SLAM polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

D-SLAM polypeptides, and preferably the secreted form, can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the D-SLAM polypeptides may be glycosylated or may be non-glycosylated. In addition, D-SLAM polypeptides may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., D-SLAM coding sequence), and/or to include genetic material (e.g., heterologous polynucleotide sequences) that is operably associated with D-SLAM polynucleotides of the invention, and which activates, alters, and/or amplifies endogenous D-SLAM polynucleotides. For example, techniques known in the art may be used to operably associate heterologous control regions (e.g., promoter and/or enhancer) and endogenous D-SLAM polynucleotide sequences via homologous recombination (see, e.g., U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989), the disclosures of each of which are incorporated by reference in their entireties).

Uses of the D-SLAM Polynucleotides

The D-SLAM polynucleotides identified herein can be used in numerous ways as reagents. The following description should be considered exemplary and utilizes known techniques.

There exists an ongoing need to identify new chromosome markers, since few chromosome marking reagents, based on actual sequence data (repeat polymorphisms), are presently available.

Briefly, sequences can be mapped to chromosomes by preparing PCR primers (preferably 15-25 bp) from the sequences shown in SEQ ID NO:1. Primers can be selected using computer analysis so that primers do not span more than one predicted exon in the genomic DNA. These primers are then used for PCR screening of somatic cell hybrids containing individual human chromosomes. Only those hybrids containing the human D-SLAM gene corresponding to the SEQ ID NO:1 will yield an amplified fragment.

Similarly, somatic hybrids provide a rapid method of PCR mapping the polynucleotides to particular chromosomes. Three or more clones can be assigned per day using a single thermal cycler. Moreover, sublocalization of the D-SLAM polynucleotides can be achieved with panels of specific chromosome fragments. Other gene mapping strategies that can be used include in situ hybridization, prescreening with labeled flow-sorted chromosomes, and preselection by hybridization to construct chromosome specific-cDNA libraries.

Precise chromosomal location of the D-SLAM polynucleotides can also be achieved using fluorescence in situ hybridization (FISH) of a metaphase chromosomal spread. This technique uses polynucleotides as short as 500 or 600 bases; however, polynucleotides 2,000-4,000 bp are preferred. For a review of this technique, see Verma et al., “Human Chromosomes: a Manual of Basic Techniques,” Pergamon Press, New York (1988).

For chromosome mapping, the D-SLAM polynucleotides can be used individually (to mark a single chromosome or a single site on that chromosome) or in panels (for marking multiple sites and/or multiple chromosomes). Preferred polynucleotides correspond to the noncoding regions of the cDNAs because the coding sequences are more likely conserved within gene families, thus increasing the chance of cross hybridization during chromosomal mapping.

Once a polynucleotide has been mapped to a precise chromosomal location, the physical position of the polynucleotide can be used in linkage analysis. Linkage analysis establishes coinheritance between a chromosomal location and presentation of a particular disease. (Disease mapping data are found, for example, in V. McKusick, Mendelian Inheritance in Man (available on line through Johns Hopkins University Welch Medical Library).) Assuming 1 megabase mapping resolution and one gene per 20 kb, a cDNA precisely localized to a chromosomal region associated with the disease could be one of 50-500 potential causative genes.

Thus, once coinheritance is established, differences in the D-SLAM polynucleotide and the corresponding gene between affected and unaffected individuals can be examined. First, visible structural alterations in the chromosomes, such as deletions or translocations, are examined in chromosome spreads or by PCR. If no structural alterations exist, the presence of point mutations are ascertained. Mutations observed in some or all affected individuals, but not in normal individuals, indicates that the mutation may cause the disease. However, complete sequencing of the D-SLAM polypeptide and the corresponding gene from several normal individuals is required to distinguish the mutation from a polymorphism. If a new polymorphism is identified, this polymorphic polypeptide can be used for further linkage analysis.

Furthermore, increased or decreased expression of the gene in affected individuals as compared to unaffected individuals can be assessed using D-SLAM polynucleotides. Any of these alterations (altered expression, chromosomal rearrangement, or mutation) can be used as a diagnostic or prognostic marker.**

In addition to the foregoing, a D-SLAM polynucleotide can be used to control gene expression through triple helix formation or antisense DNA or RNA. Both methods rely on binding of the polynucleotide to DNA or RNA. For these techniques, preferred polynucleotides are usually 20 to 40 bases in length and complementary to either the region of the gene involved in transcription (triple helix—see Lee et al., Nucl. Acids Res. 6:3073 (1979); Cooney et al., Science 241:456 (1988); and Dervan et al., Science 251:1360 (1991)) or to the mRNA itself (antisense—Okano, J. Neurochem. 56:560 (1991); Oligodeoxy-nucleotides as Antisense Inhibitors of Gene Expression, CRC Press, Boca Raton, Fla. (1988).) Triple helix formation optimally results in a shut-off of RNA transcription from DNA, while antisense RNA hybridization blocks translation of an mRNA molecule into polypeptide. Both techniques are effective in model systems, and the information disclosed herein can be used to design antisense or triple helix polynucleotides in an effort to treat disease.

D-SLAM polynucleotides are also useful in gene therapy. One goal of gene therapy is to insert a normal gene into an organism having a defective gene, in an effort to correct the genetic defect. D-SLAM offers a means of targeting such genetic defects in a highly accurate manner. Another goal is to insert a new gene that was not present in the host genome, thereby producing a new trait in the host cell.

The D-SLAM polynucleotides are also useful for identifying individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identifying personnel. This method does not suffer from the current limitations of “Dog Tags” which can be lost, switched, or stolen, making positive identification difficult. The D-SLAM polynucleotides can be used as additional DNA markers for RFLP.

The D-SLAM polynucleotides can also be used as an alternative to RFLP, by determining the actual base-by-base DNA sequence of selected portions of an individual's genome. These sequences can be used to prepare PCR primers for amplifying and isolating such selected DNA, which can then be sequenced. Using this technique, individuals can be identified because each individual will have a unique set of DNA sequences. Once an unique ID database is established for an individual, positive identification of that individual, living or dead, can be made from extremely small tissue samples.

Forensic biology also benefits from using DNA-based identification techniques as disclosed herein. DNA sequences taken from very small biological samples such as tissues, e.g., hair or skin, or body fluids, e.g., blood, saliva, semen, etc., can be amplified using PCR. In one prior art technique, gene sequences amplified from polymorphic loci, such as DQa class II HLA gene, are used in forensic biology to identify individuals. (Erlich, H., PCR Technology, Freeman and Co. (1992).) Once these specific polymorphic loci are amplified, they are digested with one or more restriction enzymes, yielding an identifying set of bands on a Southern blot probed with DNA corresponding to the DQa class II HLA gene. Similarly, D-SLAM polynucleotides can be used as polymorphic markers for forensic purposes.

There is also a need for reagents capable of identifying the source of a particular tissue. Such need arises, for example, in forensics when presented with tissue of unknown origin. Appropriate reagents can comprise, for example, DNA probes or primers specific to particular tissue prepared from D-SLAM sequences. Panels of such reagents can identify tissue by species and/or by organ type. In a similar fashion, these reagents can be used to screen tissue cultures for contamination.

Because D-SLAM is found expressed in dendritic cells, T cell lymphoma, lymph node, spleen, thymus, small intestine, and uterus, D-SLAM polynucleotides are useful as hybridization probes for differential identification of the tissue(s) or cell type(s) present in a biological sample. Similarly, polypeptides and antibodies directed to D-SLAM polypeptides are useful to provide immunological probes for differential identification of the tissue(s) or cell type(s). In addition, for a number of disorders of the above tissues or cells, particularly of the immune system, significantly higher or lower levels of D-SLAM gene expression may be detected in certain tissues (e.g., cancerous and wounded tissues) or bodily fluids (e.g., serum, plasma, urine, synovial fluid or spinal fluid) taken from an individual having such a disorder, relative to a “standard” D-SLAM gene expression level, i.e., the D-SLAM expression level in healthy tissue from an individual not having the immune system disorder.

Thus, the invention provides a diagnostic method of a disorder, which involves: (a) assaying D-SLAM gene expression level in cells or body fluid of an individual; (b) comparing the D-SLAM gene expression level with a standard D-SLAM gene expression level, whereby an increase or decrease in the assayed D-SLAM gene expression level compared to the standard expression level is indicative of disorder in the immune system.

In the very least, the D-SLAM polynucleotides can be used as molecular weight markers on Southern gels, as diagnostic probes for the presence of a specific mRNA in a particular cell type, as a probe to “subtract-out” known sequences in the process of discovering novel polynucleotides, for selecting and making oligomers for attachment to a “gene chip” or other support, to raise anti-DNA antibodies using DNA immunization techniques, and as an antigen to elicit an immune response.

Uses of D-SLAM Polypeptides

D-SLAM polypeptides can be used in numerous ways. The following description should be considered exemplary and utilizes known techniques.

D-SLAM polypeptides can be used to assay protein levels in a biological sample using antibody-based techniques. For example, protein expression in tissues can be studied with classical immunohistological methods. (Jalkanen, M., et al., J. Cell. Biol. 101:976-985 (1985); Jalkanen, M., et al., J. Cell. Biol. 105:3087-3096 (1987).) Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (125I, 121I), carbon (14C), sulfur (35S), tritium (3H), indium (112In), and technetium (99mTc), and fluorescent labels, such as fluorescein and rhodamine, and biotin.

In addition to assaying secreted protein levels in a biological sample, proteins can also be detected in vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A protein-specific antibody or antibody fragment which has been labeled with an appropriate detectable imaging moiety, such as a radioisotope (for example, 131I, 112In, 99mTc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).)

Thus, the invention provides a diagnostic method of a disorder, which involves (a) assaying the expression of D-SLAM polypeptide in cells or body fluid of an individual; (b) comparing the level of gene expression with a standard gene expression level, whereby an increase or decrease in the assayed D-SLAM polypeptide gene expression level compared to the standard expression level is indicative of a disorder.

Moreover, D-SLAM polypeptides can be used to treat disease. For example, patients can be administered D-SLAM polypeptides in an effort to replace absent or decreased levels of the D-SLAM polypeptide (e.g., insulin), to supplement absent or decreased levels of a different polypeptide (e.g., hemoglobin S for hemoglobin B), to inhibit the activity of a polypeptide (e.g., an oncogene), to activate the activity of a polypeptide (e.g., by binding to a receptor), to reduce the activity of a membrane bound receptor by competing with it for free ligand (e.g., soluble TNF receptors used in reducing inflammation), or to bring about a desired response (e.g., blood vessel growth).

Similarly, antibodies directed to D-SLAM polypeptides can also be used to treat disease. For example, administration of an antibody directed to a D-SLAM polypeptide can bind and reduce overproduction of the polypeptide. Similarly, administration of an antibody can activate the polypeptide, such as by binding to a polypeptide bound to a membrane (receptor).

At the very least, the D-SLAM polypeptides can be used as molecular weight markers on SDS-PAGE gels or on molecular sieve gel filtration columns using methods well known to those of skill in the art. D-SLAM polypeptides can also be used to raise antibodies, which in turn are used to measure protein expression from a recombinant cell, as a way of assessing transformation of the host cell. Moreover, D-SLAM polypeptides can be used to test the following biological activities.

Biological Activities of D-SLAM

D-SLAM polynucleotides and polypeptides can be used in assays to test for one or more biological activities. If D-SLAM polynucleotides and polypeptides do exhibit activity in a particular assay, it is likely that D-SLAM may be involved in the diseases associated with the biological activity. Therefore, D-SLAM could be used to treat the associated disease.

D-SLAM is a cell surface receptor homologous to members of the Secreted Lymphocyte Activation Molecule (SLAM) family, and thus should have activity similar to other SLAM family members. Current studies in the literature demonstrate that SLAM can associate with itself, and that this homotypic interaction can activate B- and T-cells. Therefore, D-SLAM may interact specifically with SLAM, with D-SLAM (a homotypic interaction), or other B- and T-cell receptor molecules on the surface of B- and T-cells to affect the activation, proliferation, survival, and/or differentiation of immune cells. Similarly, soluble D-SLAM may be an important costimulatory molecule for therapeutic uses or immune modulation. Ligands, such as antibodies, may mimic the action of soluble D-SLAM by binding to D-SLAM, SLAM, or other dendritic cell receptors.

Binding of D-SLAM induces the production of interferon-gamma from other cell types, particularly T- and B-cells (data not shown.) The binding may occur through homotypic association with membrane bound D-SLAM, association with SLAM, or association with other T- or B-cell receptors. Ligands, such as antibodies, may mimic the induction of interferon-gamma by soluble D-SLAM by binding to D-SLAM, SLAM, or other dendritic cell receptors.

Moreover, because of the tissue distribution of D-SLAM, this protein may also play a role in stimulating dendritic or antigen presenting cells. For example, a secreted form of D-SLAM, containing the extracellular domain or the full-length form, may bind to and stimulate D-SLAM molecules located on the surface of dendritic or antigen-presenting cells in homotypic manner. Binding may also occur to SLAM, or other dendritic cell surface receptors. This binding may regulate the survival, proliferation, differentiation, activation or maturation of dendritic cells or antigen presenting cells, effecting antigen recognition and immune response. Moreover, ligands, such as antibodies, may mimic the action of soluble D-SLAM by binding to D-SLAM, SLAM, or other dendritic cell receptors.

Thus, D-SLAM may be useful as a therapeutic molecule. It could be used to control the proliferation, activation, maturation, survival, and/or differentiation of hematopoietic cells, in particular B- and T-cells. Particularly, D-SLAM may be a useful therapeutic to mediate immune modulation, and may influence the Th0-TH1-TH2 profile of a patient's immune system. For example, D-SLAM may drive immune response to the Th0-TH1 pathway. This control of immune cells would be particularly important in the treatment of immune disorders, such as autoimmune diseases or immunosuppression (see below). Preferably, treatment of immune disorders could be carried out using a secreted form of D-SLAM, gene therapy, or ex vivo applications. Moreover, inhibitors of D-SLAM, either blocking antibodies or mutant forms, could modulate the expression of D-SLAM. These inhibitors may be useful to treat diseases associated with the misregulation of D-SLAM, such as T cell lymphoma.

Immune Activity

D-SLAM polypeptides or polynucleotides may be useful in treating deficiencies or disorders of the immune system, by activating or inhibiting the proliferation, differentiation, or mobilization (chemotaxis) of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune deficiencies or disorders may be genetic, somatic, such as cancer or some autoimmune disorders, acquired (e.g., by chemotherapy or toxins), or infectious. Moreover, D-SLAM polynucleotides or polypeptides can be used as a marker or detector of a particular immune system disease or disorder.

D-SLAM polynucleotides or polypeptides may be useful in treating or detecting deficiencies or disorders of hematopoietic cells. D-SLAM polypeptides or polynucleotides could be used to increase differentiation and proliferation of hematopoietic cells, including the pluripotent stem cells, in an effort to treat those disorders associated with a decrease in certain (or many) types hematopoietic cells. Examples of immunologic deficiency syndromes include, but are not limited to: blood protein disorders (e.g. agammaglobulinemia, dysgammaglobulinemia), ataxia telangiectasia, common variable immunodeficiency, Digeorge Syndrome, HIV infection, HRLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

Moreover, D-SLAM polypeptides or polynucleotides can also be used to modulate hemostatic (the stopping of bleeding) or thrombolytic activity (clot formation). For example, by increasing hemostatic or thrombolytic activity, D-SLAM polynucleotides or polypeptides could be used to treat blood coagulation disorders (e.g., afibrinogenemia, factor deficiencies), blood platelet disorders (e.g. thrombocytopenia), or wounds resulting from trauma, surgery, or other causes. Alternatively, D-SLAM polynucleotides or polypeptides that can decrease hemostatic or thrombolytic activity could be used to inhibit or dissolve clotting, important in the treatment of heart attacks (infarction), strokes, or scarring.

D-SLAM polynucleotides or polypeptides may also be useful in treating or detecting autoimmune disorders. Many autoimmune disorders result from inappropriate recognition of self as foreign material by immune cells. This inappropriate recognition results in an immune response leading to the destruction of the host tissue. Therefore, the administration of D-SLAM polypeptides or polynucleotides that can inhibit an immune response, particularly the proliferation, differentiation, or chemotaxis of T-cells, may be an effective therapy in preventing autoimmune disorders.

Examples of autoimmune disorders that can be treated or detected by D-SLAM include, but are not limited to: Addison's Disease, hemolytic anemia, antiphospholipid syndrome, rheumatoid arthritis, dermatitis, allergic encephalomyelitis, glomerulonephritis, Goodpasture's Syndrome, Graves' Disease, Multiple Sclerosis, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, and autoimmune inflammatory eye disease.

Similarly, allergic reactions and conditions, such as asthma (particularly allergic asthma) or other respiratory problems, may also be treated by D-SLAM polypeptides or polynucleotides. Moreover, D-SLAM can be used to treat anaphylaxis, hypersensitivity to an antigenic molecule, or blood group incompatibility.

D-SLAM polynucleotides or polypeptides may also be used to treat and/or prevent organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of D-SLAM polypeptides or polynucleotides that inhibits an immune response, particularly the proliferation, differentiation, or chemotaxis of T-cells, may be an effective therapy in preventing organ rejection or GVHD.

Similarly, D-SLAM polypeptides or polynucleotides may also be used to modulate inflammation. For example, D-SLAM polypeptides or polynucleotides may inhibit the proliferation and differentiation of cells involved in an inflammatory response. These molecules can be used to treat inflammatory conditions, both chronic and acute conditions, including inflammation associated with infection (e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease, Crohn's disease, or resulting from over production of cytokines (e.g., TNF or IL-1.)

Hyperproliferative Disorders

D-SLAM polypeptides or polynucleotides can be used to treat or detect hyperproliferative disorders, including neoplasms. D-SLAM polypeptides or polynucleotides may inhibit the proliferation of the disorder through direct or indirect interactions. Alternatively, D-SLAM polypeptides or polynucleotides may proliferate other cells which can inhibit the hyperproliferative disorder.

For example, by increasing an immune response, particularly increasing antigenic qualities of the hyperproliferative disorder or by proliferating, differentiating, or mobilizing T-cells, hyperproliferative disorders can be treated. This immune response may be increased by either enhancing an existing immune response, or by initiating a new immune response. Alternatively, decreasing an immune response may also be a method of treating hyperproliferative disorders, such as a chemotherapeutic agent.

Examples of hyperproliferative disorders that can be treated or detected by D-SLAM polynucleotides or polypeptides include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Similarly, other hyperproliferative disorders can also be treated or detected by D-SLAM polynucleotides or polypeptides. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

Infectious Disease

D-SLAM polypeptides or polynucleotides can be used to treat or detect infectious agents. For example, by increasing the immune response, particularly increasing the proliferation and differentiation of B and/or T cells, infectious diseases may be treated. The immune response may be increased by either enhancing an existing immune response, or by initiating a new immune response. Alternatively, D-SLAM polypeptides or polynucleotides may also directly inhibit the infectious agent, without necessarily eliciting an immune response.

Viruses are one example of an infectious agent that can cause disease or symptoms that can be treated or detected by D-SLAM polynucleotides or polypeptides. Examples of viruses, include, but are not limited to the following DNA and RNA viral families: Arbovirus, Adenoviridae, Arenaviridae, Arterivirus, Birnaviridae, Bunyaviridae, Caliciviridae, Circoviridae, Coronaviridae, Flaviviridae, Hepadnaviridae (Hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza), Papovaviridae, Parvoviridae, Picornaviridae, Poxviridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiolitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposi's, warts), and viremia. D-SLAM polypeptides or polynucleotides can be used to treat or detect any of these symptoms or diseases.

Similarly, bacterial or fungal agents that can cause disease or symptoms and that can be treated or detected by D-SLAM polynucleotides or polypeptides include, but not limited to, the following Gram-Negative and Gram-positive bacterial families and fungi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g., Acinetobacter, Gonorrhea, Menigococcal), Pasteurellacea Infections (e.g., Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections. D-SLAM polypeptides or polynucleotides can be used to treat or detect any of these symptoms or diseases.

Moreover, parasitic agents causing disease or symptoms that can be treated or detected by D-SLAM polynucleotides or polypeptides include, but not limited to, the following families: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, and Trichomonas. These parasites can cause a variety of diseases or symptoms, including, but not limited to: Scabies, Trombiculiasis, eye infections, intestinal disease (e.g., dysentery, giardiasis), liver disease, lung disease, opportunistic infections (e.g., AIDS related), Malaria, pregnancy complications, and toxoplasmosis. D-SLAM polypeptides or polynucleotides can be used to treat or detect any of these symptoms or diseases.

Preferably, treatment using D-SLAM polypeptides or polynucleotides could either be by administering an effective amount of D-SLAM polypeptide to the patient, or by removing cells from the patient, supplying the cells with D-SLAM polynucleotide, and returning the engineered cells to the patient (ex vivo therapy). Moreover, the D-SLAM polypeptide or polynucleotide can be used as an antigen in a vaccine to raise an immune response against infectious disease.

Regeneration

D-SLAM polynucleotides or polypeptides can be used to differentiate, proliferate, and attract cells, leading to the regeneration of tissues. (See, Science 276:59-87 (1997).) The regeneration of tissues could be used to repair, replace, or protect tissue damaged by congenital defects, trauma (wounds, burns, incisions, or ulcers), age, disease (e.g. osteoporosis, osteocarthritis, periodontal disease, liver failure), surgery, including cosmetic plastic surgery, fibrosis, reperfusion injury, or systemic cytokine damage.

Tissues that could be regenerated using the present invention include organs (e.g., pancreas, liver, intestine, kidney, skin, endothelium), muscle (smooth, skeletal or cardiac), vasculature (including vascular and lymphatics), nervous, hematopoietic, and skeletal (bone, cartilage, tendon, and ligament) tissue. Preferably, regeneration occurs without or decreased scarring. Regeneration also may include angiogenesis.

Moreover, D-SLAM polynucleotides or polypeptides may increase regeneration of tissues difficult to heal. For example, increased tendon/ligament regeneration would quicken recovery time after damage. D-SLAM polynucleotides or polypeptides of the present invention could also be used prophylactically in an effort to avoid damage. Specific diseases that could be treated include of tendinitis, carpal tunnel syndrome, and other tendon or ligament defects. A further example of tissue regeneration of non-healing wounds includes pressure ulcers, ulcers associated with vascular insufficiency, surgical, and traumatic wounds.

Similarly, nerve and brain tissue could also be regenerated by using D-SLAM polynucleotides or polypeptides to proliferate and differentiate nerve cells. Diseases that could be treated using this method include central and peripheral nervous system diseases, neuropathies, or mechanical and traumatic disorders (e.g., spinal cord disorders, head trauma, cerebrovascular disease, and stoke). Specifically, diseases associated with peripheral nerve injuries, peripheral neuropathy (e.g., resulting from chemotherapy or other medical therapies), localized neuropathies, and central nervous system diseases (e.g., Alzheimer's disease, Parkinson's disease, Huntington's disease, amyotrophic lateral sclerosis, and Shy-Drager syndrome), could all be treated using the D-SLAM polynucleotides or polypeptides.

Chemotaxis

D-SLAM polynucleotides or polypeptides may have chemotaxis activity. A chemotaxic molecule attracts or mobilizes cells (e.g., monocytes, fibroblasts, neutrophils, T-cells, mast cells, eosinophils, epithelial and/or endothelial cells) to a particular site in the body, such as inflammation, infection, or site of hyperproliferation. The mobilized cells can then fight off and/or heal the particular trauma or abnormality.

D-SLAM polynucleotides or polypeptides may increase chemotaxic activity of particular cells. These chemotactic molecules can then be used to treat inflammation, infection, hyperproliferative disorders, or any immune system disorder by increasing the number of cells targeted to a particular location in the body. For example, chemotaxic molecules can be used to treat wounds and other trauma to tissues by attracting immune cells to the injured location. As a chemotactic molecule, D-SLAM could also attract fibroblasts, which can be used to treat wounds.

It is also contemplated that D-SLAM polynucleotides or polypeptides may inhibit chemotactic activity. These molecules could also be used to treat disorders. Thus, D-SLAM polynucleotides or polypeptides could be used as an inhibitor of chemotaxis.

Binding Activity

D-SLAM polypeptides may be used to screen for molecules that bind to D-SLAM or for molecules to which D-SLAM binds. The binding of D-SLAM and the molecule may activate (agonist), increase, inhibit (antagonist), or decrease activity of the D-SLAM or the molecule bound. Examples of such molecules include antibodies, oligonucleotides, proteins (e.g., receptors), or small molecules.

Preferably, the molecule is closely related to the natural ligand of D-SLAM, e.g., a fragment of the ligand, or a natural substrate, a ligand, a structural or functional mimetic. (See, Coligan et al., Current Protocols in Immunology 1(2):Chapter 5 (1991).) Similarly, the molecule can be closely related to the natural receptor to which D-SLAM binds, or at least, a fragment of the receptor capable of being bound by D-SLAM (e.g., active site). In either case, the molecule can be rationally designed using known techniques.

Preferably, the screening for these molecules involves producing appropriate cells which express D-SLAM, either as a secreted protein or on the cell membrane. Preferred cells include cells from mammals, yeast, Drosophila, or E. coli. Cells expressing D-SLAM (or cell membrane containing the expressed polypeptide) are then preferably contacted with a test compound potentially containing the molecule to observe binding, stimulation, or inhibition of activity of either D-SLAM or the molecule.

The assay may simply test binding of a candidate compound to D-SLAM, wherein binding is detected by a label, or in an assay involving competition with a labeled competitor. Further, the assay may test whether the candidate compound results in a signal generated by binding to D-SLAM.

Alternatively, the assay can be carried out using cell-free preparations, polypeptide/molecule affixed to a solid support, chemical libraries, or natural product mixtures. The assay may also simply comprise the steps of mixing a candidate compound with a solution containing D-SLAM, measuring D-SLAM/molecule activity or binding, and comparing the D-SLAM/molecule activity or binding to a standard.

Preferably, an ELISA assay can measure D-SLAM level or activity in a sample (e.g., biological sample) using a monoclonal or polyclonal antibody. The antibody can measure D-SLAM level or activity by either binding, directly or indirectly, to D-SLAM or by competing with D-SLAM for a substrate.

All of these above assays can be used as diagnostic or prognostic markers. The molecules discovered using these assays can be used to treat disease or to bring about a particular result in a patient (e.g., blood vessel growth) by activating or inhibiting the D-SLAM/molecule. Moreover, the assays can discover agents which may inhibit or enhance the production of D-SLAM from suitably manipulated cells or tissues.

Therefore, the invention includes a method of identifying compounds which bind to D-SLAM comprising the steps of: (a) incubating a candidate binding compound with D-SLAM; and (b) determining if binding has occurred. Moreover, the invention includes a method of identifying agonists/antagonists comprising the steps of: (a) incubating a candidate compound with D-SLAM, (b) assaying a biological activity, and (b) determining if a biological activity of D-SLAM has been altered.

Other Activities

D-SLAM polypeptides or polynucleotides may also increase or decrease the differentiation or proliferation of embryonic stem cells, besides, as discussed above, hematopoietic lineage.

D-SLAM polypeptides or polynucleotides may also be used to modulate mammalian characteristics, such as body height, weight, hair color, eye color, skin, percentage of adipose tissue, pigmentation, size, and shape (e.g., cosmetic surgery). Similarly, D-SLAM polypeptides or polynucleotides may be used to modulate mammalian metabolism affecting catabolism, anabolism, processing, utilization, and storage of energy.

D-SLAM polypeptides or polynucleotides may be used to change a mammal's mental state or physical state by influencing biorhythms, caricadic rhythms, depression (including depressive disorders), tendency for violence, tolerance for pain, reproductive capabilities (preferably by Activin or Inhibin-like activity), hormonal or endocrine levels, appetite, libido, memory, stress, or other cognitive qualities.

D-SLAM polypeptides or polynucleotides may also be used as a food additive or preservative, such as to increase or decrease storage capabilities, fat content, lipid, protein, carbohydrate, vitamins, minerals, cofactors or other nutritional components.

Having generally described the invention, the same will be more readily understood by reference to the following examples, which are provided by way of illustration and are not intended as limiting.

### EXAMPLES

### Example 1

### Isolation of the D-SLAM cDNA Clone from the Deposited Sample

The cDNA for D-SLAM is inserted into the SalI/NotI multiple cloning site of pCMVSport 3.0. (Life Technologies, Inc., P.O. Box 6009, Gaithersburg, Md. 20897.) pCMVSport 3.0 contains an ampicillin resistance gene and may be transformed into E. coli strain DH10B, also available from Life Technologies. (See, for instance, Gruber, C. E., et al., Focus 15:59- (1993).)

Two approaches can be used to isolate D-SLAM from the deposited sample. First, a specific polynucleotide of SEQ ID NO:1 with 30-40 nucleotides is synthesized using an Applied Biosystems DNA synthesizer according to the sequence reported. The oligonucleotide is labeled, for instance, with ³²P-γ-ATP using T4 polynucleotide kinase and purified according to routine methods. (E.g., Maniatis et al., Molecular Cloning: A Laboratory Manual, Cold Spring Harbor Press, Cold Spring, N.Y. (1982).) The plasmid mixture is transformed into a suitable host (such as XL-1 Blue (Stratagene)) using techniques known to those of skill in the art, such as those provided by the vector supplier or in related publications or patents. The transformants are plated on 1.5% agar plates (containing the appropriate selection agent, e.g., ampicillin) to a density of about 150 transformants (colonies) per plate. These plates are screened using Nylon membranes according to routine methods for bacterial colony screening (e.g., Sambrook et al., Molecular Cloning: A Laboratory Manual, 2nd Edit., (1989), Cold Spring Harbor Laboratory Press, pages 1.93 to 1.104), or other techniques known to those of skill in the art.

Alternatively, two primers of 17-20 nucleotides derived from both ends of the SEQ ID NO:1 (i.e., within the region of SEQ ID NO:1 bounded by the 5′ NT and the 3′ NT of the clone) are synthesized and used to amplify the D-SLAM cDNA using the deposited cDNA plasmid as a template. The polymerase chain reaction is carried out under routine conditions, for instance, in 25 μl of reaction mixture with 0.5 ug of the above cDNA template. A convenient reaction mixture is 1.5-5 mM MgCl₂, 0.01% (w/v) gelatin, 20 μM each of dATP, dCTP, dGTP, dTTP, 25 pmol of each primer and 0.25 Unit of Taq polymerase. Thirty five cycles of PCR (denaturation at 94 degree C. for 1 min; annealing at 55 degree C. for 1 min; elongation at 72 degree C. for 1 min) are performed with a Perkin-Elmer Cetus automated thermal cycler. The amplified product is analyzed by agarose gel electrophoresis and the DNA band with expected molecular weight is excised and purified. The PCR product is verified to be the selected sequence by subcloning and sequencing the DNA product.

Several methods are available for the identification of the 5′ or 3′ non-coding portions of the D-SLAM gene which may not be present in the deposited clone. These methods include but are not limited to, filter probing, clone enrichment using specific probes, and protocols similar or identical to 5′ and 3′ “RACE” protocols which are well known in the art. For instance, a method similar to 5′ RACE is available for generating the missing 5′ end of a desired full-length transcript. (Fromont-Racine et al., Nucleic Acids Res. 21(7):1683-1684 (1993).)

Briefly, a specific RNA oligonucleotide is ligated to the 5′ ends of a population of RNA presumably containing full-length gene RNA transcripts. A primer set containing a primer specific to the ligated RNA oligonucleotide and a primer specific to a known sequence of the D-SLAM gene of interest is used to PCR amplify the 5′ portion of the D-SLAM full-length gene. This amplified product may then be sequenced and used to generate the full length gene.

This above method starts with total RNA isolated from the desired source, although poly-A+ RNA can be used. The RNA preparation can then be treated with phosphatase if necessary to eliminate 5′ phosphate groups on degraded or damaged RNA which may interfere with the later RNA ligase step. The phosphatase should then be inactivated and the RNA treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase.

This modified RNA preparation is used as a template for first strand cDNA synthesis using a gene specific oligonucleotide. The first strand synthesis reaction is used as a template for PCR amplification of the desired 5′ end using a primer specific to the ligated RNA oligonucleotide and a primer specific to the known sequence of the gene of interest. The resultant product is then sequenced and analyzed to confirm that the 5′ end sequence belongs to the D-SLAM gene.

### Example 2

### Isolation of D-SLAM Genomic Clones

A human genomic P1 library (Genomic Systems, Inc.) is screened by PCR using primers selected for the cDNA sequence corresponding to SEQ ID NO:1, according to the method described in Example 1. (See also, Sambrook.)

### Example 3

### Tissue Distribution of D-SLAM Polypeptides

Tissue distribution of mRNA expression of D-SLAM is determined using protocols for Northern blot analysis, described by, among others, Sambrook et al. For example, a D-SLAM probe produced by the method described in Example 1 is labeled with P³² using the rediprime™ DNA labeling system (Amersham Life Science), according to manufacturer's instructions. After labeling, the probe is purified using CHROMA SPIN-100™ column (Clontech Laboratories, Inc.), according to manufacturer's protocol number PT1200-1. The purified labeled probe is then used to examine various human tissues for mRNA expression.

Multiple Tissue Northern (MTN) blots containing various human tissues (H) or human immune system tissues (IM) (Clontech) are examined with the labeled probe using ExpressHyb™ hybridization solution (Clontech) according to manufacturer's protocol number PT1190-1. Following hybridization and washing, the blots are mounted and exposed to film at −70 degree C. overnight, and the films developed according to standard procedures.

### Example 4

### Chromosomal Mapping of D-SLAM

An oligonucleotide primer set is designed according to the sequence at the 5′ end of SEQ ID NO:1. This primer preferably spans about 100 nucleotides. This primer set is then used in a polymerase chain reaction under the following set of conditions: 30 seconds, 95 degree C.; 1 minute, 56 degree C.; 1 minute, 70 degree C. This cycle is repeated 32 times followed by one 5 minute cycle at 70 degree C. Human, mouse, and hamster DNA is used as template in addition to a somatic cell hybrid panel containing individual chromosomes or chromosome fragments (Bios, Inc). The reactions is analyzed on either 8% polyacrylamide gels or 3.5% agarose gels. Chromosome mapping is determined by the presence of an approximately 100 bp PCR fragment in the particular somatic cell hybrid.

### Example 5

### Bacterial Expression of D-SLAM

D-SLAM polynucleotide encoding a D-SLAM polypeptide invention is amplified using PCR oligonucleotide primers corresponding to the 5′ and 3′ ends of the DNA sequence, as outlined in Example 1, to synthesize insertion fragments. The primers used to amplify the cDNA insert should preferably contain restriction sites, such as BamHI and XbaI, at the 5′ end of the primers in order to clone the amplified product into the expression vector. For example, BamHI and XbaI correspond to the restriction enzyme sites on the bacterial expression vector pQE-9. (Qiagen, Inc., Chatsworth, Calif.). This plasmid vector encodes antibiotic resistance (Amp^{r}), a bacterial origin of replication (ori), an IPTG-regulatable promoter/operator (P/O), a ribosome binding site (RBS), a 6-histidine tag (6-His), and restriction enzyme cloning sites.

The pQE-9 vector is digested with BamHI and XbaI and the amplified fragment is ligated into the pQE-9 vector maintaining the reading frame initiated at the bacterial RBS. The ligation mixture is then used to transform the E. coli strain M15/rep4 (Qiagen, Inc.) which contains multiple copies of the plasmid pREP4, which expresses the lacI repressor and also confers kanamycin resistance (Kan^{r}). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 ug/ml) and Kan (25 ug/ml). The O/N culture is used to inoculate a large culture at a ratio of 1:100 to 1:250. The cells are grown to an optical density 600 (O.D.⁶⁰⁰) of between 0.4 and 0.6. IPTG (Isopropyl-B-D-thiogalactopyranoside) is then added to a final concentration of 1 mM. IPTG induces by inactivating the lacI repressor, clearing the P/O leading to increased gene expression.

Cells are grown for an extra 3 to 4 hours. Cells are then harvested by centrifugation (20 rains at 6000×g). The cell pellet is solubilized in the chaotropic agent 6 Molar Guanidine HCl by stirring for 3-4 hours at 4 degree C. The cell debris is removed by centrifugation, and the supernatant containing the polypeptide is loaded onto a nickel-nitrilo-tri-acetic acid (“Ni-NTA”) affinity resin column (available from QIAGEN, Inc., supra). Proteins with a 6×His tag bind to the Ni-NTA resin with high affinity and can be purified in a simple one-step procedure (for details see: The QIAexpressionist (1995) QIAGEN, Inc., supra).

Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified D-SLAM protein is then renatured by dialyzing it against phosphate-buffered saline (PBS) or 50 mM Na-acetate, pH 6 buffer plus 200 mM NaCl. Alternatively, the D-SLAM protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins are eluted by the addition of 250 mM immidazole. Immidazole is removed by a final dialyzing step against PBS or 50 mM sodium acetate pH 6 buffer plus 200 mM NaCl. The purified D-SLAM protein is stored at 4 degree C. or frozen at −80 degree C.

In addition to the above expression vector, the present invention further includes an expression vector comprising phage operator and promoter elements operatively linked to a D-SLAM polynucleotide, called pHE4a. (ATCC™ Accession Number 209645, deposited Feb. 25, 1998.) This vector contains: 1) a neomycinphosphotransferase gene as a selection marker, 2) an E. coli origin of replication, 3) a T5 phage promoter sequence, 4) two lac operator sequences, 5) a Shine-Delgarno sequence, and 6) the lactose operon repressor gene (lacIq). The origin of replication (oriC) is derived from pUC19 (LTI, Gaithersburg, Md.). The promoter sequence and operator sequences are made synthetically.

DNA can be inserted into the pHEa by restricting the vector with NdeI and XbaI, BamHI, XhoI, or Asp718, running the restricted product on a gel, and isolating the larger fragment (the stuffer fragment should be about 310 base pairs). The DNA insert is generated according to the PCR protocol described in Example 1, using PCR primers having restriction sites for NdeI (5′ primer) and XbaI, BamHI, XhoI, or Asp718 (3′ primer). The PCR insert is gel purified and restricted with compatible enzymes. The insert and vector are ligated according to standard protocols.

The engineered vector could easily be substituted in the above protocol to express protein in a bacterial system.

### Example 6

### Purification of D-SLAM Polypeptide from an Inclusion Body

The following alternative method can be used to purify D-SLAM polypeptide expressed in E coli when it is present in the form of inclusion bodies. Unless otherwise specified, all of the following steps are conducted at 4-10 degree C.

Upon completion of the production phase of the E. coli fermentation, the cell culture is cooled to 4-10 degree C. and the cells harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfuidics, Corp. or APV Gaulin, Inc.) twice at 4000-6000 psi. The homogenate is then mixed with NaCl solution to a final concentration of 0.5 M NaCl, followed by centrifugation at 7000×g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000×g centrifugation for 15 min., the pellet is discarded and the polypeptide containing supernatant is incubated at 4 degree C. overnight to allow further GuHCl extraction.

Following high speed centrifugation (30,000×g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4 degree C. without mixing for 12 hours prior to further purification steps.

To clarify the refolded polypeptide solution, a previously prepared tangential filtration unit equipped with 0.16 um membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the D-SLAM polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the polypeptide (determined, for instance, by 16% SDS-PAGE) are then pooled.

The resultant D-SLAM polypeptide should exhibit greater than 95% purity after the above refolding and purification steps. No major contaminant bands should be observed from Commassie blue stained 16% SDS-PAGE gel when 5 ug of purified protein is loaded. The purified D-SLAM protein can also be tested for endotoxin/LPS contamination, and typically the LPS content is less than 0.1 ng/ml according to LAL assays.

### Example 7

### Cloning and Expression of D-SLAM in a Baculovirus Expression System

In this example, the plasmid shuttle vector pA2 is used to insert D-SLAM polynucleotide into a baculovirus to express D-SLAM. This expression vector contains the strong polyhedrin promoter of the Autographa californica nuclear polyhedrosis virus (AcMNPV) followed by convenient restriction sites such as BamHI, Xba I and Asp718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned D-SLAM polynucleotide.

Many other baculovirus vectors can be used in place of the vector above, such as pAc373, pVL941, and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

Specifically, the D-SLAM cDNA sequence contained in the deposited clone, including the AUG initiation codon and any naturally associated leader sequence, is amplified using the PCR protocol described in Example 1. If the naturally occurring signal sequence is used to produce the secreted protein, the pA2 vector does not need a second signal peptide. Alternatively, the vector can be modified (pA2 GP) to include a baculovirus leader sequence, using the standard methods described in Summers et al., “A Manual of Methods for Baculovirus Vectors and Insect Cell Culture Procedures,” Texas Agricultural Experimental Station Bulletin No. 1555 (1987).

The amplified fragment is isolated from a 1% agarose gel using a commercially available kit (“Geneclean,” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five ug of a plasmid containing the polynucleotide is co-transfected with 1.0 ug of a commercially available linearized baculovirus DNA (“BaculoGold™ baculovirus DNA”, Pharmingen, San Diego, Calif.), using the lipofection method described by Felgner et al., Proc. Natl. Acad. Sci. USA 84:7413-7417 (1987). One ug of BaculoGold™ virus DNA and 5 ug of the plasmid are mixed in a sterile well of a microtiter plate containing 50 ul of serum-free Grace's medium (Life Technologies Inc., Gaithersburg, Mo.). Afterwards, 10 ul Lipofectin plus 90 ul Grace's medium are added, mixed and incubated for 15 minutes at room temperature. Then the transfection mixture is added drop-wise to Sf9 insect cells (ATCC™ CRL 1711) seeded in a 35 mm tissue culture plate with 1 ml Grace's medium without serum. The plate is then incubated for 5 hours at 27 degrees C. The transfection solution is then removed from the plate and 1 ml of Grace's insect medium supplemented with 10% fetal calf serum is added. Cultivation is then continued at 27 degrees C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 ul of Grace's medium and the suspension containing the recombinant baculovirus is used to infect Sf9 cells seeded in 35 mm dishes. Four days later the supernatants of these culture dishes are harvested and then they are stored at 4 degree C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 uCi of ³⁵S-methionine and 5 uCi ³⁵S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the produced D-SLAM protein.

### Example 8

### Expression of D-SLAM in Mammalian Cells

D-SLAM polypeptide can be expressed in a mammalian cell. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC™ 37152), pSV2DHFR (ATCC™ 37146), pBC12MI (ATCC™ 67109), pCMVSport 2.0, and pCMVSport 3.0. Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, D-SLAM polypeptide can be expressed in stable cell lines containing the D-SLAM polynucleotide integrated into a chromosome. The co-transfection with a selectable marker such as DHFR, gpt, neomycin, hygromycin allows the identification and isolation of the transfected cells.

The transfected D-SLAM gene can also be amplified to express large amounts of the encoded protein. The DHFR (dihydrofolate reductase) marker is useful in developing cell lines that carry several hundred or even several thousand copies of the gene of interest. (See, e.g., Alt, F. W., et al., J. Biol. Chem. 253:1357-1370 (1978); Hamlin, J. L. and Ma, C., Biochem. et Biophys. Acta, 1097:107-143 (1990); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10:169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

Derivatives of the plasmid pSV2-DHFR (ATCC™ Accession No. 37146), the expression vectors pC4 (ATCC™ Accession No. 209646) and pC6 (ATCC™ Accession No. 209647) contain the strong promoter (LTR) of the Rous Sarcoma Virus (Cullen et al., Molecular and Cellular Biology, 438-447 (March, 1985)) plus a fragment of the CMV-enhancer (Boshart et al., Cell 41:521-530 (1985).) Multiple cloning sites, e.g., with the restriction enzyme cleavage sites BamHI, XbaI and Asp718, facilitate the cloning of D-SLAM. The vectors also contain the 3′ intron, the polyadenylation and termination signal of the rat preproinsulin gene, and the mouse DHFR gene under control of the SV40 early promoter.

Specifically, the plasmid pC6 or pC4 is digested appropriate restriction enzymes and then dephosphorylated using calf intestinal phosphates by procedures known in the art. The vector is then isolated from a 1% agarose gel.

D-SLAM polynucleotide is amplified according to the protocol outlined in Example 1. If a naturally occurring signal sequence is used to produce a secreted protein, the vector does not need a second signal peptide. Alternatively, if a naturally occurring signal sequence is not used, the vector can be modified to include a heterologous signal sequence in an effort to secrete the protein from the cell. (See, e.g., WO 96/34891.)

The amplified fragment is isolated from a 1% agarose gel using a commercially available kit (“Geneclean,” BIO 101 Inc., La Jolla, Calif.). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligated with T4 DNA ligase. E. coli BB 101 or XL-1 Blue cells are then transformed and bacteria are identified that contain the fragment inserted into plasmid pC6 or pC4 using, for instance, restriction enzyme analysis.

Chinese hamster ovary cells lacking an active DEER gene is used for transfection. Five jug of the expression plasmid pC6 or pC4 is cotransfected with 0.5 ug of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of methotrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 uM, 2 uM, 5 uM, 10 mM, 20 mM). The same procedure is repeated until clones are obtained which grow at a concentration of 100-200 uM. Expression of D-SLAM is analyzed, for instance, by SDS-PAGE and Western blot or by reversed phase HPLC analysis.

### Example 9

### Construction of N-Terminal and/or C-Terminal Deletion Mutants

The following general approach may be used to clone a N-terminal or C-terminal deletion D-SLAM deletion mutant. Generally, two oligonucleotide primers of about 15-25 nucleotides are derived from the desired 5′ and 3′ positions of a polynucleotide of SEQ ID NO:1. The 5′ and 3′ positions of the primers are determined based on the desired D-SLAM polynucleotide fragment. An initiation and stop codon are added to the 5′ and 3′ primers respectively, if necessary, to express the D-SLAM polypeptide fragment encoded by the polynucleotide fragment. Preferred D-SLAM polynucleotide fragments are those encoding the N-terminal and C-terminal deletion mutants disclosed above in the “Polynucleotide and Polypeptide Fragments” section of the Specification.

Additional nucleotides containing restriction sites to facilitate cloning of the D-SLAM polynucleotide fragment in a desired vector may also be added to the 5′ and 3′ primer sequences. The D-SLAM polynucleotide fragment is amplified from genomic DNA or from the deposited cDNA clone using the appropriate PCR oligonucleotide primers and conditions discussed herein or known in the art. The D-SLAM polypeptide fragments encoded by the D-SLAM polynucleotide fragments of the present invention may be expressed and purified in the same general manner as the full length polypeptides, although routine modifications may be necessary due to the differences in chemical and physical properties between a particular fragment and full length polypeptide.

As a means of exemplifying but not limiting the present invention, the polynucleotide encoding the D-SLAM polypeptide fragment Leu-35 to Thr-276 is amplified and cloned as follows: A 5′ primer is generated comprising a restriction enzyme site followed by an initiation codon in frame with the polynucleotide sequence encoding the N-terminal portion of the polypeptide fragment beginning with Leu-35. A complementary 3′ primer is generated comprising a restriction enzyme site followed by a stop codon in frame with the polynucleotide sequence encoding C-terminal portion of the D-SLAM polypeptide fragment ending with Thr-276.

The amplified polynucleotide fragment and the expression vector are digested with restriction enzymes which recognize the sites in the primers. The digested polynucleotides are then ligated together. The D-SLAM polynucleotide fragment is inserted into the restricted expression vector, preferably in a manner which places the D-SLAM polypeptide fragment coding region downstream from the promoter. The ligation mixture is transformed into competent E. coli cells using standard procedures and as described in the Examples herein. Plasmid DNA is isolated from resistant colonies and the identity of the cloned DNA confirmed by restriction analysis, PCR and DNA sequencing.

### Example 10

### Protein Fusions of D-SLAM

D-SLAM polypeptides are preferably fused to other proteins. These fusion proteins can be used for a variety of applications. For example, fusion of D-SLAM polypeptides to His-tag, HA-tag, protein A, IgG domains, and maltose binding protein facilitates purification. (See Example 5; see also EP A 394,827; Traunecker, et al., Nature 331:84-86 (1988).) Similarly, fusion to IgG-1, IgG-3, and albumin increases the halflife time in vivo. Nuclear localization signals fused to D-SLAM polypeptides can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein. All of the types of fusion proteins described above can be made by modifying the following protocol, which outlines the fusion of a polypeptide to an IgG molecule, or the protocol described in Example 5.

Briefly, the human Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector.

For example, if pC4 (Accession No. 209646) is used, the human Fc portion can be ligated into the BamHI cloning site. Note that the 3′ BamHI site should be destroyed. Next, the vector containing the human Fc portion is re-restricted with BamHI, linearizing the vector, and D-SLAM polynucleotide, isolated by the PCR protocol described in Example 1, is ligated into this BamHI site. Note that the polynucleotide is cloned without a stop codon, otherwise a fusion protein will not be produced.

If the naturally occurring signal sequence is used to produce the secreted protein, pC4 does not need a second signal peptide. Alternatively, if the naturally occurring signal sequence is not used, the vector can be modified to include a heterologous signal sequence. (See, e.g., WO 96/34891.)

Human IgG Fc Region:

(SEQ ID NO:4)

GGGATCCGGAGCCCAAATCTTCTGACAAAACTCACACATGCCCACCGTGC
CCAGCACCTGAATTCGAGGGTGCACCGTCAGTCTTCCTCTTCCCCCCAAA
ACCCAAGGACACCCTCATGATCTCCCGGACTCCTGAGGTCACATGCGTGG
TGGTGGACGTAAGCCACGAAGACCCTGAGGTCAAGTTCAACTGGTACGTG
GACGGCGTGGAGGTGCATAATGCCAAGACAAAGCCGCGGGAGGAGCAGTA
CAACAGCACGTACCGTGTGGTCAGCGTCCTCACCGTCCTGCACCAGGACT
GGCTGAATGGCAAGGAGTACAAGTGCAAGGTCTCCAACAAAGCCCTCCCA
ACCCCCATCGAGAAAACCATCTCCAAAGCCAAAGGGCAGCCCCGAGAACC
ACAGGTGTACACCCTGCCCCCATCCCGGGATGAGCTGACCAAGAACCAGG
TCAGCCTGACCTGCCTGGTCAAAGGCTTCTATCCAAGCGACATCGCCGTG
GAGTGGGAGAGCAATGGGCAGCCGGAGAACAACTACAAGACCACGCCTCC
CGTGCTGGACTCCGACGGCTCCTTCTTCCTCTACAGCAAGCTCACCGTGG
ACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGG
TAAATGAGTGCGACGGCCGCGACTCTAGAGGAT

### Example 11

### Production of an Antibody

The antibodies of the present invention can be prepared by a variety of methods. (See, Current Protocols, Chapter 2.) For example, cells expressing D-SLAM is administered to an animal to induce the production of sera containing polyclonal antibodies. In a preferred method, a preparation of D-SLAM protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal

antibodies can be prepared using hybridoma technology. (Köhler et al., Nature 256:495 (1975); Köhler et al., Eur. J. Immunol. 6:511 (1976); Köhler et al., Eur. J. Immunol. 6:292 (1976); Hammerling et al., in: Monoclonal Antibodies and T-Cell Hybridomas, Elsevier, N.Y., pp. 563-681 (1981).) In general, such procedures involve immunizing an animal (preferably a mouse) with D-SLAM polypeptide or, more preferably, with a secreted D-SLAM polypeptide-expressing cell. Such cells may be cultured in any suitable tissue culture medium; however, it is preferable to culture cells in Earle's modified Eagle's medium supplemented with 10% fetal bovine serum (inactivated at about 56 degree C.), and supplemented with about 10 g/l of nonessential amino acids, about 1,000 U/ml of penicillin, and about 100 ug/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP2O), available from the ATCC™. After fusion, the resulting hybridoma cells are selectively maintained in HAT medium, and then cloned by limiting dilution as described by Wands et al. (Gastroenterology 80:225-232 (1981).) The hybridoma cells obtained through such a selection are then assayed to identify clones which secrete antibodies capable of binding the D-SLAM polypeptide.

Alternatively, additional antibodies capable of binding to D-SLAM polypeptide can be produced in a two-step procedure using anti-idiotypic antibodies. Such a method makes use of the fact that antibodies are themselves antigens, and therefore, it is possible to obtain an antibody which binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the D-SLAM protein-specific antibody can be blocked by D-SLAM. Such antibodies comprise anti-idiotypic antibodies to the D-SLAM protein-specific antibody and can be used to immunize an animal to induce formation of further D-SLAM protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, secreted D-SLAM protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

### Example 12

### Production of D-SLAM Protein for High-Throughput Screening Assays

The following protocol produces a supernatant containing D-SLAM polypeptide to be tested. This supernatant can then be used in the Screening Assays described in Examples 14-21.

First, dilute Poly-D-Lysine (644 587 Boehringer-Mannheim) stock solution (1 mg/ml in PBS) 1:20 in PBS (w/o calcium or magnesium 17-516F Biowhittaker) for a working solution of 50 ug/ml. Add 200 ul of this solution to each well (24 well plates) and incubate at RT for 20 minutes. Be sure to distribute the solution over each well (note: a 12-channel pipetter may be used with tips on every other channel). Aspirate off the Poly-D-Lysine solution and rinse with 1 ml PBS (Phosphate Buffered Saline). The PBS should remain in the well until just prior to plating the cells and plates may be poly-lysine coated in advance for up to two weeks.

Plate 293T cells (do not carry cells past P+20) at 2×10⁵ cells/well in 0.5 ml DMEM (Dulbecco's Modified Eagle Medium) (with 4.5 G/L glucose and L-glutamine (12-604F Biowhittaker))/10% heat inactivated FBS (14-503F Biowhittaker)/1×Penstrep (17-602E Biowhittaker). Let the cells grow overnight.

The next day, mix together in a sterile solution basin: 300 ul Lipofectamine (18324-012 Gibco/BRL) and 5 ml Optimem I (31985070 Gibco/BRL)/96-well plate. With a small volume multi-channel pipetter, aliquot approximately 2 ug of an expression vector containing a polynucleotide insert, produced by the methods described in Examples 8-10, into an appropriately labeled 96-well round bottom plate. With a multi-channel pipetter, add 50 ul of the Lipofectamine/Optimem I mixture to each well. Pipette up and down gently to mix. Incubate at RT 15-45 minutes. After about 20 minutes, use a multi-channel pipetter to add 150 ul Optimem I to each well. As a control, one plate of vector DNA lacking an insert should be transfected with each set of transfections.

Preferably, the transfection should be performed by tag-teaming the following tasks. By tag-teaming, hands on time is cut in half, and the cells do not spend too much time on PBS. First, person A aspirates off the media from four 24-well plates of cells, and then person B rinses each well with 0.5-1 ml PBS. Person A then aspirates off PBS rinse, and person B, using a 12-channel pipetter with tips on every other channel, adds the 200 ul of DNA/Lipofectamine/Optimem I complex to the odd wells first, then to the even wells, to each row on the 24-well plates. Incubate at 37 degree C. for 6 hours.

While cells are incubating, prepare appropriate media, either 1% BSA in DMEM with 1×penstrep, or HGS CHO-5 media (116.6 mg/L of CaCl2 (anhyd); 0.00130 mg/L CuSO₄-5H₂O; 0.050 mg/L of Fe(NO₃)₃-9H₂O; 0.417 mg/L of FeSO₄-7H₂O; 311.80 mg/L of Kcl; 28.64 mg/L of MgCl₂; 48.84 mg/L of MgSO₄; 6995.50 mg/L of NaCl; 2400.0 mg/L of NaHCO₃; 62.50 mg/L of NaH₂PO₄—H₂0; 71.02 mg/L of Na₂HPO4; 0.4320 mg/L of ZnSO₄-7H₂O; 0.002 mg/L of Arachidonic Acid; 1.022 mg/L of Cholesterol; 0.070 mg/L of DL-alpha-Tocopherol-Acetate; 0.0520 mg/L of Linoleic Acid; 0.010 mg/L of Linolenic Acid; 0.010 mg/L of Myristic Acid; 0.010 mg/L of Oleic Acid; 0.010 mg/L of Palmitric Acid; 0.010 mg/L of Palmitic Acid; 100 mg/L of Pluronic F-68; 0.010 mg/L of Stearic Acid; 2.20 mg/L of Tween 80; 4551 mg/L of D-Glucose; 130.85 mg/ml of L-Alanine; 147.50 mg/ml of L-Arginine-HCL; 7.50 mg/ml of L-Asparagine-H₂0; 6.65 mg/ml of L-Aspartic Acid; 29.56 mg/ml of L-Cystine-2HCL-H₂0; 31.29 mg/ml of L-Cystine-2HCL; 7.35 mg/ml of L-Glutamic Acid; 365.0 mg/ml of L-Glutamine; 18.75 mg/ml of Glycine; 52.48 mg/ml of L-Histidine-HCL-H₂0; 106.97 mg/ml of L-Isoleucine; 111.45 mg/ml of L-Leucine; 163.75 mg/ml of L-Lysine HCL; 32.34 mg/ml of L-Methionine; 68.48 mg/ml of L-Phenylalanine; 40.0 mg/ml of L-Proline; 26.25 mg/ml of L-Serine; 101.05 mg/ml of L-Threonine; 19.22 mg/ml of L-Tryptophan; 91.79 mg/ml of L-Tyrosine-2Na-2H₂0; and 99.65 mg/ml of L-Valine; 0.0035 mg/L of Biotin; 3.24 mg/L of D-Ca Pantothenate; 11.78 mg/L of Choline Chloride; 4.65 mg/L of Folic Acid; 15.60 mg/L of i-Inositol; 3.02 mg/L of Niacinamide; 3.00 mg/L of Pyridoxal HCL; 0.031 mg/L of Pyridoxine HCL; 0.319 mg/L of Riboflavin; 3.17 mg/L of Thiamine HCL; 0.365 mg/L of Thymidine; 0.680 mg/L of Vitamin B₁₂; 25 mM of HEPES Buffer; 2.39 mg/L of Na Hypoxanthine; 0.105 mg/L of Lipoic Acid; 0.081 mg/L of Sodium Putrescine-2HCL; 55.0 mg/L of Sodium Pyruvate; 0.0067 mg/L of Sodium Selenite; 20 uM of Ethanolamine; 0.122 mg/L of Ferric Citrate; 41.70 mg/L of Methyl-B-Cyclodextrin complexed with Linoleic Acid; 33.33 mg/L of Methyl-B-Cyclodextrin complexed with Oleic Acid; 10 mg/L of Methyl-B-Cyclodextrin complexed with Retinal Acetate. Adjust osmolarity to 327 mOsm) with 2 mm glutamine and 1×penstrep. (BSA (81-068-3 Bayer) 100 gm dissolved in 1 L DMEM for a 10% BSA stock solution). Filter the media and collect 50 ul for endotoxin assay in 15 ml polystyrene conical.

The transfection reaction is terminated, preferably by tag-teaming, at the end of the incubation period. Person A aspirates off the transfection media, while person B adds 1.5 ml appropriate media to each well. Incubate at 37 degree C. for 45 or 72 hours depending on the media used: 1% BSA for 45 hours or CHO-5 for 72 hours.

On day four, using a 300 ul multichannel pipetter, aliquot 600 ul in one 1 ml deep well plate and the remaining supernatant into a 2 ml deep well. The supernatants from each well can then be used in the assays described in Examples 14-21.

It is specifically understood that when activity is obtained in any of the assays described below using a supernatant, the activity originates from either the D-SLAM polypeptide directly (e.g., as a secreted protein) or by D-SLAM inducing expression of other proteins, which are then secreted into the supernatant. Thus, the invention further provides a method of identifying the protein in the supernatant characterized by an activity in a particular assay.

### Example 13

### Construction of GAS Reporter Construct

One signal transduction pathway involved in the differentiation and proliferation of cells is called the Jaks-STATs pathway. Activated proteins in the Jaks-STATs pathway bind to gamma activation site “GAS” elements or interferon-sensitive responsive element (“ISRE”), located in the promoter of many genes. The binding of a protein to these elements alter the expression of the associated gene.

GAS and ISRE elements are recognized by a class of transcription factors called Signal Transducers and Activators of Transcription, or “STATs.” There are six members of the STATs family. Stat1 and Stat3 are present in many cell types, as is Stat2 (as response to IFN-alpha is widespread). Stat4 is more restricted and is not in many cell types though it has been found in T helper class I, cells after treatment with IL-12. Stat5 was originally called mammary growth factor, but has been found at higher concentrations in other cells including myeloid cells. It can be activated in tissue culture cells by many cytokines.

The STATs are activated to translocate from the cytoplasm to the nucleus upon tyrosine phosphorylation by a set of kinases known as the Janus Kinase (“Jaks”) family. Jaks represent a distinct family of soluble tyrosine kinases and include Tyk2, Jak1, Jak2, and Jak3. These kinases display significant sequence similarity and are generally catalytically inactive in resting cells.

The Jaks are activated by a wide range of receptors summarized in the Table below. (Adapted from review by Schidler and Darnell, Ann. Rev. Biochem. 64:621-51 (1995).) A cytokine receptor family, capable of activating Jaks, is divided into two groups: (a) Class 1 includes receptors for IL-2, IL-3, IL-4, IL-6, IL-7, IL-9, IL-11, IL-12, IL-15, Epo, PRL, GH, G-CSF, GM-CSF, LIF, CNTF, and thrombopoietin; and (b) Class 2 includes IFN-a, IFN-g, and IL-10. The Class 1 receptors share a conserved cysteine motif (a set of four conserved cysteines and one tryptophan) and a WSXWS motif (a membrane proxial region encoding Trp-Ser-Xxx-Trp-Ser (SEQ ID NO:5)).

Thus, on binding of a ligand to a receptor, Jaks are activated, which in turn activate STATs, which then translocate and bind to GAS elements. This entire process is encompassed in the Jaks-STATs signal transduction pathway.

Therefore, activation of the Jaks-STATs pathway, reflected by the binding of the GAS or the ISRE element, can be used to indicate proteins involved in the proliferation and differentiation of cells. For example, growth factors and cytokines are known to activate the Jaks-STATs pathway. (See Table below.) Thus, by using GAS elements linked to reporter molecules, activators of the Jaks-STATs pathway can be identified.

| | JAKs | | | | | |
|---|---|---|---|---|---|---|
| Ligand | tyk2 | Jak1 | Jak2 | Jak3 | STATS | GAS(elements) or ISRE |
| IFN family | | | | | | |
| IFN-a/B | + | + | − | − | 1, 2, 3 | ISRE |
| IFN-g | | + | + | − | 1 | GAS (IRF1 > Lys6 > IFP) |
| Il-10 | + | ? | ? | − | 1, 3 | |
| gp130 family | | | | | | |
| IL-6 (Pleiotrohic) | + | + | + | ? | 1, 3 | GAS (IRF1 > Lys6 > IFP) |
| Il-11 (Pleiotrohic) | ? | + | ? | ? | 1, 3 | |
| OnM (Pleiotrohic) | ? | + | + | ? | 1, 3 | |
| LIF (Pleiotrohic) | ? | + | + | ? | 1, 3 | |
| CNTF (Pleiotrohic) | −/+ | + | + | ? | 1, 3 | |
| G-CSF (Pleiotrohic) | ? | + | ? | ? | 1, 3 | |
| IL-12 (Pleiotrohic) | + | − | + | + | 1, 3 | |
| g-C family | | | | | | |
| IL-2 (lymphocytes) | − | + | − | + | 1, 3, 5 | GAS |
| IL-4 (lymph/myeloid) | − | + | − | + | 6 | GAS (IRF1 = IFP >> Ly6)(IgH) |
| IL-7 (lymphocytes) | − | + | − | + | 5 | GAS |
| IL-9 (lymphocytes) | − | + | − | + | 5 | GAS |
| IL-13 (lymphocyte) | − | + | ? | ? | 6 | GAS |
| IL-15 | ? | + | ? | + | 5 | GAS |
| gp140 family | | | | | | |
| IL-3 (myeloid) | − | − | + | − | 5 | GAS (IRF1 > IFP >> Ly6) |
| IL-5 (myeloid) | − | − | + | − | 5 | GAS |
| GM-CSF (myeloid) | − | − | + | − | 5 | GAS |
| Growth hormone family | | | | | | |
| GH | ? | − | + | − | 5 | |
| PRL | ? | +/− | + | − | 1, 3, 5 | |
| EPO | ? | − | + | − | 5 | GAS (B-CAS > IRF1 = IFP >> Ly6) |
| Receptor Tyrosine Kinases | | | | | | |
| EGF | ? | + | + | − | 1, 3 | GAS (IRF1) |
| PDGF | ? | + | + | − | 1, 3 | |
| CSF-1 | ? | + | + | − | 1, 3 | GAS (not IRF1) |

To construct a synthetic GAS containing promoter element, which is used in the Biological Assays described in Examples 14-15, a PCR based strategy is employed to generate a GAS-SV40 promoter sequence. The 5′ primer contains four tandem copies of the GAS binding site found in the IRF1 promoter and previously demonstrated to bind STATs upon induction with a range of cytokines (Rothman et al., Immunity 1:457-468 (1994).), although other GAS or ISRE elements can be used instead. The 5′ primer also contains 18 bp of sequence complementary to the SV40 early promoter sequence and is flanked with an XhoI site. The sequence of the 5′ primer is:

(SEQ ID NO:6)

5′:GCGCCTCGAGATTTCCCCGAAATCTAGATTTCCCCGAAATGATTTCC
CCGAAATGATTTCCCCGAAATATCTGCCATCTCAATTAG:3′

The downstream primer is complementary to the SV40 promoter and is flanked with a Hind III site: 5′:GCGGCAAGCTTTTTGCAAAGCCTAGGC:3′ (SEQ ID NO:7)

PCR amplification is performed using the SV40 promoter template present in the B-gal:promoter plasmid obtained from Clontech. The resulting PCR fragment is digested with XhoI/Hind III and subcloned into BLSK2−. (Stratagene.) Sequencing with forward and reverse primers confirms that the insert contains the following sequence:

(SEQ ID NO:8)

5′:CTCGAGATTTCCCCGAAATCTAGATTTCCCCGAAATGATTTCCCCGA
AATGATTTCCCCGAAATATCTGCCATCTCAATTAGTCAGCAACCATAGTC
CCGCCCCTAACTCCGCCCATCCCGCCCCTAACTCCGCCCAGTTCCGCCCA
TTCTCCGCCCCATGGCTGACTAATTTTTTTTATTTATGCAGAGGCCGAGG
CCGCCTCGGCCTCTGAGCTATTCCAGAAGTAGTGAGGAGCTTTTTTGGAG
GCCTAGGCTTTTGCAAAAAGCTT:3′

With this GAS promoter element linked to the SV40 promoter, a GAS:SEAP2 reporter construct is next engineered. Here, the reporter molecule is a secreted alkaline phosphatase, or “SEAP.” Clearly, however, any reporter molecule can be instead of SEAP, in this or in any of the other Examples. Well known reporter molecules that can be used instead of SEAP include chloramphenicol acetyltransferase (CAT), luciferase, alkaline phosphatase, B-galactosidase, green fluorescent protein (GFP), or any protein detectable by an antibody.

The above sequence confirmed synthetic GAS-SV40 promoter element is subcloned into the pSEAP-Promoter vector obtained from Clontech using HindIII and XhoI, effectively replacing the SV40 promoter with the amplified GAS:SV40 promoter element, to create the GAS-SEAP vector. However, this vector does not contain a neomycin resistance gene, and therefore, is not preferred for mammalian expression systems.

Thus, in order to generate mammalian stable cell lines expressing the GAS-SEAP reporter, the GAS-SEAP cassette is removed from the GAS-SEAP vector using SalI and NotI, and inserted into a backbone vector containing the neomycin resistance gene, such as pGFP-1 (Clontech), using these restriction sites in the multiple cloning site, to create the GAS-SEAP/Neo vector. Once this vector is transfected into mammalian cells, this vector can then be used as a reporter molecule for GAS binding as described in Examples 14-15.

Other constructs can be made using the above description and replacing GAS with a different promoter sequence. For example, construction of reporter molecules containing NFK-B and EGR promoter sequences are described in Examples 16 and 17. However, many other promoters can be substituted using the protocols described in these Examples. For instance, SRE, IL-2, NFAT, or Osteocalcin promoters can be substituted, alone or in combination (e.g., GAS/NF-KB/EGR, GAS/NF-KB, Il-2/NFAT, or NF-KB/GAS). Similarly, other cell lines can be used to test reporter construct activity, such as HELA (epithelial), HUVEC (endothelial), Reh (B-cell), Saos-2 (osteoblast), HUVAC (aortic), or Cardiomyocyte.

### Example 14

### High-Throughput Screening Assay for T-Cell Activity

The following protocol is used to assess T-cell activity of D-SLAM by determining whether D-SLAM supernatant proliferates and/or differentiates T-cells. T-cell activity is assessed using the GAS/SEAP/Neo construct produced in Example 13. Thus, factors that increase SEAP activity indicate the ability to activate the Jaks-STATS signal transduction pathway. The T-cell used in this assay is Jurkat T-cells (ATCC™ Accession No. TIB-152), although Molt-3 cells (ATCC™ Accession No. CRL-1552) and Molt-4 cells (ATCC™ Accession No. CRL-1582) cells can also be used.

Jurkat T-cells are lymphoblastic CD4+ Th1 helper cells. In order to generate stable cell lines, approximately 2 million Jurkat cells are transfected with the GAS-SEAP/neo vector using DMRIE-C (Life Technologies) (transfection procedure described below). The transfected cells are seeded to a density of approximately 20,000 cells per well and transfectants resistant to 1 mg/ml genticin selected. Resistant colonies are expanded and then tested for their response to increasing concentrations of interferon gamma. The dose response of a selected clone is demonstrated.

Specifically, the following protocol will yield sufficient cells for 75 wells containing 200 ul of cells. Thus, it is either scaled up, or performed in multiple to generate sufficient cells for multiple 96 well plates. Jurkat cells are maintained in RPMI+10% serum with 1% Pen-Strep. Combine 2.5 mls of OPTI-MEM (Life Technologies) with 10 ug of plasmid DNA in a T25 flask. Add 2.5 ml OPTI-MEM containing 50 ul of DMRIE-C and incubate at room temperature for 15-45 mins.

During the incubation period, count cell concentration, spin down the required number of cells (10⁷ per transfection), and resuspend in OPTI-MEM to a final concentration of 10⁷ cells/ml. Then add 1 ml of 1×10⁷ cells in OPTI-MEM to T25 flask and incubate at 37 degree C. for 6 hrs. After the incubation, add 10 ml of RPMI+15% serum.

The Jurkat:GAS-SEAP stable reporter lines are maintained in RPMI+10% serum, 1 mg/ml Genticin, and 1% Pen-Strep. These cells are treated with supernatants containing D-SLAM polypeptides or D-SLAM induced polypeptides as produced by the protocol described in Example 12.

On the day of treatment with the supernatant, the cells should be washed and resuspended in fresh RPMI+10% serum to a density of 500,000 cells per ml. The exact number of cells required will depend on the number of supernatants being screened. For one 96 well plate, approximately 10 million cells (for 10 plates, 100 million cells) are required.

Transfer the cells to a triangular reservoir boat, in order to dispense the cells into a 96 well dish, using a 12 channel pipette. Using a 12 channel pipette, transfer 200 ul of cells into each well (therefore adding 100,000 cells per well).

After all the plates have been seeded, 50 ul of the supernatants are transferred directly from the 96 well plate containing the supernatants into each well using a 12 channel pipette. In addition, a dose of exogenous interferon gamma (0.1, 1.0, 10 ng) is added to wells H9, H10, and H11 to serve as additional positive controls for the assay.

The 96 well dishes containing Jurkat cells treated with supernatants are placed in an incubator for 48 hrs (note: this time is variable between 48-72 hrs). 35 ul samples from each well are then transferred to an opaque 96 well plate using a 12 channel pipette. The opaque plates should be covered (using cellophane covers) and stored at −20 degree C. until SEAP assays are performed according to Example 18. The plates containing the remaining treated cells are placed at 4 degree C. and serve as a source of material for repeating the assay on a specific well if desired.

As a positive control, 100 Unit/ml interferon gamma can be used which is known to activate Jurkat T cells. Over 30 fold induction is typically observed in the positive control wells.

### Example 15

### High-Throughput Screening Assay Identifying Myeloid Activity

The following protocol is used to assess myeloid activity of D-SLAM by determining whether D-SLAM proliferates and/or differentiates myeloid cells. Myeloid cell activity is assessed using the GAS/SEAP/Neo construct produced in Example 13. Thus, factors that increase SEAP activity indicate the ability to activate the Jaks-STATS signal transduction pathway. The myeloid cell used in this assay is U937, a pre-monocyte cell line, although TF-1, HL60, or KG1 can be used.

To transiently transfect U937 cells with the GAS/SEAP/Neo construct produced in Example 13, a DEAE-Dextran method (Kharbanda et. al., 1994, Cell Growth & Differentiation, 5:259-265) is used. First, harvest 2×10e⁷ U937 cells and wash with PBS. The U937 cells are usually grown in RPMI 1640 medium containing 10% heat-inactivated fetal bovine serum (FBS) supplemented with 100 units/ml penicillin and 100 mg/ml streptomycin.

Next, suspend the cells in 1 ml of 20 mM Tris-HCl (pH 7.4) buffer containing 0.5 mg/ml DEAE-Dextran, 8 ug GAS-SEAP2 plasmid DNA, 140 mM NaCl, 5 mM KCl, 375 uM Na₂HPO₄.7H₂O, 1 mM MgCl₂, and 675 uM CaCl₂. Incubate at 37 degree C. for 45 min.

Wash the cells with RPMI 1640 medium containing 10% FBS and then resuspend in 10 ml complete medium and incubate at 37 degree C. for 36 hr.

The GAS-SEAP/U937 stable cells are obtained by growing the cells in 400 ug/ml G418. The G418-free medium is used for routine growth but every one to two months, the cells should be re-grown in 400 ug/ml G418 for couple of passages.

These cells are tested by harvesting 1×10⁸ cells (this is enough for ten 96-well plates assay) and wash with PBS. Suspend the cells in 200 ml above described growth medium, with a final density of 5×10⁵ cells/ml. Plate 200 ul cells per well in the 96-well plate (or 1×10⁵ cells/well).

Add 50 ul of the supernatant prepared by the protocol described in Example 12. Incubate at 37 degree C. for 48 to 72 hr. As a positive control, 100 Unit/ml interferon gamma can be used which is known to activate U937 cells. Over 30 fold induction is typically observed in the positive control wells. SEAP assay the supernatant according to the protocol described in Example 18.

### Example 16

### High-Throughput Screening Assay Identifying Neuronal Activity

When cells undergo differentiation and proliferation, a group of genes are activated through many different signal transduction pathways. One of these genes, EGR1 (early growth response gene 1), is induced in various tissues and cell types upon activation. The promoter of EGR1 is responsible for such induction. Using the EGR1 promoter linked to reporter molecules, activation of cells can be assessed by D-SLAM.

Particularly, the following protocol is used to assess neuronal activity in PC12 cell lines. PC12 cells (rat phenochromocytoma cells) are known to proliferate and/or differentiate by activation with a number of mitogens, such as TPA (tetradecanoyl phorbol acetate), NGF (nerve growth factor), and EGF (epidermal growth factor). The EGR1 gene expression is activated during this treatment. Thus, by stably transfecting PC12 cells with a construct containing an EGR promoter linked to SEAP reporter, activation of PC12 cells by D-SLAM can be assessed.

The EGR/SEAP reporter construct can be assembled by the following protocol. The EGR-1 promoter sequence (−633 to +1) (Sakamoto K et al., Oncogene 6:867-871 (1991)) can be PCR amplified from human genomic DNA using the following primers:

(SEQ ID NO:9)

5′ GCGCTCGAGGGATGACAGCGATAGAACCCCGG-3′

(SEQ ID NO:10)

5′ GCGAAGCTTCGCGACTCCCCGGATCCGCCTC-3′

Using the GAS:SEAP/Neo vector produced in Example 13, EGR1 amplified product can then be inserted into this vector. Linearize the GAS:SEAP/Neo vector using restriction enzymes XhoI/HindIII, removing the GAS/SV40 stuffer. Restrict the EGR1 amplified product with these same enzymes. Ligate the vector and the EGR1 promoter.

To prepare 96 well-plates for cell culture, two mls of a coating solution (1:30 dilution of collagen type I (Upstate Biotech Inc. Cat#08-115) in 30% ethanol (filter sterilized)) is added per one 10 cm plate or 50 ml per well of the 96-well plate, and allowed to air dry for 2 hr.

PC12 cells are routinely grown in RPMI-1640 medium (Bio Whittaker) containing 10% horse serum (JRH BIOSCIENCES, Cat. # 12449-78P), 5% heat-inactivated fetal bovine serum (FBS) supplemented with 100 units/ml penicillin and 100 ug/ml streptomycin on a precoated 10 cm tissue culture dish. One to four split is done every three to four days. Cells are removed from the plates by scraping and resuspended with pipetting up and down for more than 15 times.

Transfect the EGR/SEAP/Neo construct into PC12 using the Lipofectamine protocol described in Example 12. EGR-SEAP/PC12 stable cells are obtained by growing the cells in 300 ug/ml G418. The G418-free medium is used for routine growth but every one to two months, the cells should be re-grown in 300 ug/ml G418 for couple of passages.

To assay for neuronal activity, a 10 cm plate with cells around 70 to 80% confluent is screened by removing the old medium. Wash the cells once with PBS (Phosphate buffered saline). Then starve the cells in low serum medium (RPMI-1640 containing 1% horse serum and 0.5% FBS with antibiotics) overnight.

The next morning, remove the medium and wash the cells with PBS. Scrape off the cells from the plate, suspend the cells well in 2 ml low serum medium. Count the cell number and add more low serum medium to reach final cell density as 5×10⁵ cells/ml.

Add 200 ul of the cell suspension to each well of 96-well plate (equivalent to 1×10⁵ cells/well). Add 50 ul supernatant produced by Example 12, 37 degree C. for 48 to 72 hr. As a positive control, a growth factor known to activate PC12 cells through EGR can be used, such as 50 ng/t of Neuronal Growth Factor (NGF). Over fifty-fold induction of SEAP is typically seen in the positive control wells. SEAP assay the supernatant according to Example 18.

### Example 17

### High-Throughput Screening Assay for T-Cell Activity

NF-KB (Nuclear Factor KB) is a transcription factor activated by a wide variety of agents including the inflammatory cytokines IL-1 and TNF, CD30 and CD40, lymphotoxin-alpha and lymphotoxin-beta, by exposure to LPS or thrombin, and by expression of certain viral gene products. As a transcription factor, NF-KB regulates the expression of genes involved in immune cell activation, control of apoptosis (NF-KB appears to shield cells from apoptosis), B and T-cell development, anti-viral and antimicrobial responses, and multiple stress responses.

In non-stimulated conditions, NF-KB is retained in the cytoplasm with I-KB (Inhibitor KB). However, upon stimulation, I-KB is phosphorylated and degraded, causing NF-KB to shuttle to the nucleus, thereby activating transcription of target genes. Target genes activated by NF-KB include IL-2, IL-6, GM-CSF, ICAM-1 and class 1 MHC.

Due to its central role and ability to respond to a range of stimuli, reporter constructs utilizing the NF-KB promoter element are used to screen the supernatants produced in Example 12. Activators or inhibitors of NF-KB would be useful in treating diseases. For example, inhibitors of NF-KB could be used to treat those diseases related to the acute or chronic activation of NF-KB, such as rheumatoid arthritis.

To construct a vector containing the NF-KB promoter element, a PCR based strategy is employed. The upstream primer contains four tandem copies of the NF-KB binding site (GGGGACTTTCCC) (SEQ ID NO:11), 18 bp of sequence complementary to the 5′ end of the SV40 early promoter sequence, and is flanked with an XhoI site:

(SEQ ID NO:12)

5′:GCGGCCTCGAGGGGACTTTCCCGGGGACTTTCCGGGGACTTTCCGGG
ACTTTCCATCCTGCCATCTCAATTAG:3′

The downstream primer is complementary to the 3′ end of the SV40 promoter and is flanked with a Hind III site:

| | |
|---|---|
| 5′:GCGGCAAGCTTTTTGCAAAGCCTAGGC:3′ | (SEQ ID NO:7) |

PCR amplification is performed using the SV40 promoter template present in the pB-gal:promoter plasmid obtained from Clontech. The resulting PCR fragment is digested with XhoI and Hind III and subcloned into BLSK2−. (Stratagene) Sequencing with the T7 and T3 primers confirms the insert contains the following sequence:

(SEQ ID NO:13)

5′:CTCGAGGGGACTTTCCCGGGGACTTTCCGGGGACTTTCCGGGACTTT
CCATCTGCCATCTCAATTAGTCAGCAACCATAGTCCCGCCCCTAACTCCG
CCCATCCCGCCCCTAACTCCGCCCAGTTCCGCCCATTCTCCGCCCCATGG
CTGACTAATTTTTTTTATTTATGCAGAGGCCGAGGCCGCCTCGGCCTCTG
AGCTATTCCAGAAGTAGTGAGGAGGCTTTTTTGGAGGCCTAGGCTTTTGC
AAAAAGCTT:3′.

Next, replace the SV40 minimal promoter element present in the pSEAP2-promoter plasmid (Clontech) with this NF-KB/SV40 fragment using XhoI and HindIII. However, this vector does not contain a neomycin resistance gene, and therefore, is not preferred for mammalian expression systems.

In order to generate stable mammalian cell lines, the NF-κB/SV40/SEAP cassette is removed from the above NF-KB/SEAP vector using restriction enzymes SalI and NotI, and inserted into a vector containing neomycin resistance. Particularly, the NF-KB/SV40/SEAP cassette was inserted into pGFP-1 (Clontech), replacing the GFP gene, after restricting pGFP-1 with SalI and NotI.

Once NF-KB/SV40/SEAP/Neo vector is created, stable Jurkat T-cells are created and maintained according to the protocol described in Example 14. Similarly, the method for assaying supernatants with these stable Jurkat T-cells is also described in Example 14. As a positive control, exogenous TNF alpha (0.1, 1, 10 ng) is added to wells H9, H10, and H11, with a 5-10 fold activation typically observed.

### Example 18

### Assay for SEAP Activity

As a reporter molecule for the assays described in Examples 14-17, SEAP activity is assayed using the Tropix Phospho-light Kit (Cat. BP-400) according to the following general procedure. The Tropix Phospho-light Kit supplies the Dilution, Assay, and Reaction Buffers used below.

Prime a dispenser with the 2.5× Dilution Buffer and dispense 15 ul of 2.5× dilution buffer into Optiplates containing 35 μl of a supernatant. Seal the plates with a plastic sealer and incubate at 65 degree C. for 30 min. Separate the Optiplates to avoid uneven heating.

Cool the samples to room temperature for 15 minutes. Empty the dispenser and prime with the Assay Buffer. Add 50 μl Assay Buffer and incubate at room temperature 5 min. Empty the dispenser and prime with the Reaction Buffer (see the table below). Add 50 ul Reaction Buffer and incubate at room temperature for 20 minutes. Since the intensity of the chemiluminescent signal is time dependent, and it takes about 10 minutes to read 5 plates on luminometer, one should treat 5 plates at each time and start the second set 10 minutes later.

Read the relative light unit in the luminometer. Set H12 as blank, and print the results. An increase in chemiluminescence indicates reporter activity.

Reaction Buffer Formulation:

| # of plates | Rxn buffer diluent (ml) | CSPD (ml) |
|---|---|---|
| 10 | 60 | 3 |
| 11 | 65 | 3.25 |
| 12 | 70 | 3.5 |
| 13 | 75 | 3.75 |
| 14 | 80 | 4 |
| 15 | 85 | 4.25 |
| 16 | 90 | 4.5 |
| 17 | 95 | 4.75 |
| 18 | 100 | 5 |
| 19 | 105 | 5.25 |
| 20 | 110 | 5.5 |
| 21 | 115 | 5.75 |
| 22 | 120 | 6 |
| 23 | 125 | 6.25 |
| 24 | 130 | 6.5 |
| 25 | 135 | 6.75 |
| 26 | 140 | 7 |
| 27 | 145 | 7.25 |
| 28 | 150 | 7.5 |
| 29 | 155 | 7.75 |
| 30 | 160 | 8 |
| 31 | 165 | 8.25 |
| 32 | 170 | 8.5 |
| 33 | 175 | 8.75 |
| 34 | 180 | 9 |
| 35 | 185 | 9.25 |
| 36 | 190 | 9.5 |
| 37 | 195 | 9.75 |
| 38 | 200 | 10 |
| 39 | 205 | 10.25 |
| 40 | 210 | 10.5 |
| 41 | 215 | 10.75 |
| 42 | 220 | 11 |
| 43 | 225 | 11.25 |
| 44 | 230 | 11.5 |
| 45 | 235 | 11.75 |
| 46 | 240 | 12 |
| 47 | 245 | 12.25 |
| 48 | 250 | 12.5 |
| 49 | 255 | 12.75 |
| 50 | 260 | 13 |

### Example 19

### High-Throughput Screening Assay Identifying Changes in Small Molecule Concentration and Membrane Permeability

Binding of a ligand to a receptor is known to alter intracellular levels of small molecules, such as calcium, potassium, sodium, and pH, as well as alter membrane potential. These alterations can be measured in an assay to identify supernatants which bind to receptors of a particular cell. Although the following protocol describes an assay for calcium, this protocol can easily be modified to detect changes in potassium, sodium, pH, membrane potential, or any other small molecule which is detectable by a fluorescent probe.

The following assay uses Fluorometric Imaging Plate Reader (“FLIPR”) to measure changes in fluorescent molecules (Molecular Probes) that bind small molecules. Clearly, any fluorescent molecule detecting a small molecule can be used instead of the calcium fluorescent molecule, fluo-3, used here.

For adherent cells, seed the cells at 10,000-20,000 cells/well in a Co-star black 96-well plate with clear bottom. The plate is incubated in a CO₂ incubator for 20 hours. The adherent cells are washed two times in Biotek washer with 200 ul of HBSS (Hank's Balanced Salt Solution) leaving 100 μl of buffer after the final wash.

A stock solution of 1 mg/ml fluo-3 is made in 10% pluronic acid DMSO. To load the cells with fluo-3, 50 ul of 12 ug/ml fluo-3 is added to each well. The plate is incubated at 37 degree C. in a CO₂ incubator for 60 min. The plate is washed four times in the Biotek washer with BSS leaving 100 ul of buffer.

For non-adherent cells, the cells are spun down from culture media. Cells are re-suspended to 2-5×10⁶ cells/ml with HBSS in a 50-ml conical tube. 4 ul of 1 mg/ml fluo-3 solution in 10% pluronic acid DMSO is added to each ml of cell suspension. The tube is then placed in a 37 degree C. water bath for 30-60 min. The cells are washed twice with HBSS, resuspended to 1×10⁶ cells/ml, and dispensed into a microplate, 100 μl/well. The plate is centrifuged at 1000 rpm for 5 min. The plate is then washed once in Denley CellWash with 200 ul, followed by an aspiration step to 100 μL final volume.

For a non-cell based assay, each well contains a fluorescent molecule, such as fluo-3. The supernatant is added to the well, and a change in fluorescence is detected.

To measure the fluorescence of intracellular calcium, the FLIPR is set for the following parameters: (1) System gain is 300-800 mW; (2) Exposure time is 0.4 second; (3) Camera F/stop is F/2; (4) Excitation is 488 nm; (5) Emission is 530 nm; and (6) Sample addition is 50 μl. Increased emission at 530 nm indicates an extracellular signaling event caused by the a molecule, either D-SLAM or a molecule induced by D-SLAM, which has resulted in an increase in the intracellular Ca⁺⁺ concentration.

### Example 20

### High-Throughput Screening Assay Identifying Tyrosine Kinase Activity

The Protein Tyrosine Kinases (PTK) represent a diverse group of transmembrane and cytoplasmic kinases. Within the Receptor Protein Tyrosine Kinase RPTK) group are receptors for a range of mitogenic and metabolic growth factors including the PDGF, FGF, EGF, NGF, HGF and Insulin receptor subfamilies. In addition there are a large family of RPTKs for which the corresponding ligand is unknown. Ligands for RPTKs include mainly secreted small proteins, but also membrane-bound and extracellular matrix proteins.

Activation of RPTK by ligands involves ligand-mediated receptor dimerization, resulting in transphosphorylation of the receptor subunits and activation of the cytoplasmic tyrosine kinases. The cytoplasmic tyrosine kinases include receptor associated tyrosine kinases of the src-family (e.g., src, yes, lck, lyn, fyn) and non-receptor linked and cytosolic protein tyrosine kinases, such as the Jak family, members of which mediate signal transduction triggered by the cytokine superfamily of receptors (e.g., the Interleukins, Interferons, GM-CSF, and Leptin).

Because of the wide range of known factors capable of stimulating tyrosine kinase activity, identifying whether D-SLAM or a molecule induced by D-SLAM is capable of activating tyrosine kinase signal transduction pathways is of interest. Therefore, the following protocol is designed to identify such molecules capable of activating the tyrosine kinase signal transduction pathways.

Seed target cells (e.g., primary keratinocytes) at a density of approximately 25,000 cells per well in a 96 well Loprodyne Silent Screen Plates purchased from Nalge Nunc (Naperville, Ill.). The plates are sterilized with two 30 minute rinses with 100% ethanol, rinsed with water and dried overnight. Some plates are coated for 2 hr with 100 ml of cell culture grade type I collagen (50 mg/ml), gelatin (2%) or polylysine (50 mg/ml), all of which can be purchased from Sigma Chemicals (St. Louis, Mo.) or 10% Matrigel purchased from Becton Dickinson (Bedford, Mass.), or calf serum, rinsed with PBS and stored at 4 degree C. Cell growth on these plates is assayed by seeding 5,000 cells/well in growth medium and indirect quantitation of cell number through use of alamarBlue as described by the manufacturer Alamar Biosciences, Inc. (Sacramento, Calif.) after 48 hr. Falcon plate covers #3071 from Becton Dickinson (Bedford, Mass.) are used to cover the Loprodyne Silent Screen Plates. Falcon Microtest III cell culture plates can also be used in some proliferation experiments.

To prepare extracts, A431 cells are seeded onto the nylon membranes of Loprodyne plates (20,000/200 ml/well) and cultured overnight in complete medium. Cells are quiesced by incubation in serum-free basal medium for 24 hr. After 5-20 minutes treatment with EGF (60 ng/ml) or 50 ul of the supernatant produced in Example 12, the medium was removed and 100 ml of extraction buffer ((20 mM HEPES pH 7.5, 0.15 M NaCl, 1% Triton X-100, 0.1% SDS, 2 mM Na3VO4, 2 mM Na4P2O7 and a cocktail of protease inhibitors (# 1836170) obtained from Boeheringer Mannheim (Indianapolis, Ind.) is added to each well and the plate is shaken on a rotating shaker for 5 minutes at 4° C. The plate is then placed in a vacuum transfer manifold and the extract filtered through the 0.45 mm membrane bottoms of each well using house vacuum. Extracts are collected in a 96-well catch/assay plate in the bottom of the vacuum manifold and immediately placed on ice. To obtain extracts clarified by centrifugation, the content of each well, after detergent solubilization for 5 minutes, is removed and centrifuged for 15 minutes at 4 degree C. at 16,000×g.

Test the filtered extracts for levels of tyrosine kinase activity. Although many methods of detecting tyrosine kinase activity are known, one method is described here.

Generally, the tyrosine kinase activity of a supernatant is evaluated by determining its ability to phosphorylate a tyrosine residue on a specific substrate (a biotinylated peptide). Biotinylated peptides that can be used for this purpose include PSK1 (corresponding to amino acids 6-20 of the cell division kinase cdc2-p34) and PSK2 (corresponding to amino acids 1-17 of gastrin). Both peptides are substrates for a range of tyrosine kinases and are available from Boehringer Mannheim.

The tyrosine kinase reaction is set up by adding the following components in order. First, add 10 ul of 5 uM Biotinylated Peptide, then 10 ul ATP/Mg₂₊ (5 mM ATP/50 mM MgCl₂), then 10 ul of 5× Assay Buffer (40 mM imidazole hydrochloride, pH7.3, 40 mM beta-glycerophosphate, 1 mM EGTA, 100 mM MgCl₂, 5 mM MnCl₂, 0.5 mg/ml BSA), then 5 ul of Sodium Vanadate (1 mM), and then 5 ul of water. Mix the components gently and preincubate the reaction mix at 30 degree C. for 2 min. Initial the reaction by adding 10 ul of the control enzyme or the filtered supernatant.

The tyrosine kinase assay reaction is then terminated by adding 10 ul of 120 mm EDTA and place the reactions on ice.

Tyrosine kinase activity is determined by transferring 50 ul aliquot of reaction mixture to a microtiter plate (MTP) module and incubating at 37 degree C. for 20 min. This allows the streptavadin coated 96 well plate to associate with the biotinylated peptide. Wash the MTP module with 300 ul/well of PBS four times. Next add 75 ul of anti-phospotyrosine antibody conjugated to horse radish peroxidase (anti-P-Tyr-POD (0.5 u/ml)) to each well and incubate at 37 degree C. for one hour. Wash the well as above.

Next add 100 ul of peroxidase substrate solution (Boehringer Mannheim) and incubate at room temperature for at least 5 mins (up to 30 min). Measure the absorbance of the sample at 405 nm by using ELISA reader. The level of bound peroxidase activity is quantitated using an ELISA reader and reflects the level of tyrosine kinase activity.

### Example 21

### High-Throughput Screening Assay Identifying Phosphorylation Activity

As a potential alternative and/or compliment to the assay of protein tyrosine kinase activity described in Example 20, an assay which detects activation (phosphorylation) of major intracellular signal transduction intermediates can also be used. For example, as described below one particular assay can detect tyrosine phosphorylation of the Erk-1 and Erk-2 kinases. However, phosphorylation of other molecules, such as Raf, JNK, p38 MAP, Map kinase kinase (MEK), MEK kinase, Src, Muscle specific kinase (MuSK), IRAK, Tec, and Janus, as well as any other phosphoserine, phosphotyrosine, or phosphothreonine molecule, can be detected by substituting these molecules for Erk-1 or Erk-2 in the following assay.

Specifically, assay plates are made by coating the wells of a 96-well ELISA plate with 0.1 ml of protein G (1 ug/ml) for 2 hr at room temp, (RT). The plates are then rinsed with PBS and blocked with 3% BSA/PBS for 1 hr at RT. The protein G plates are then treated with 2 commercial monoclonal antibodies (10 ng/well) against Erk-1 and Erk-2 (1 hr at RT) (Santa Cruz Biotechnology). (To detect other molecules, this step can easily be modified by substituting a monoclonal antibody detecting any of the above described molecules.) After 3-5 rinses with PBS, the plates are stored at 4 degree C. until use.

A431 cells are seeded at 20,000/well in a 96-well Loprodyne filterplate and cultured overnight in growth medium. The cells are then starved for 48 hr in basal medium (DMEM) and then treated with EGF (6 ng/well) or 50 ul of the supernatants obtained in Example 12 for 5-20 minutes. The cells are then solubilized and extracts filtered directly into the assay plate.

After incubation with the extract for 1 hr at RT, the wells are again rinsed. As a positive control, a commercial preparation of MAP kinase (10 ng/well) is used in place of A431 extract. Plates are then treated with a commercial polyclonal (rabbit) antibody (1 ug/ml) which specifically recognizes the phosphorylated epitope of the Erk-1 and Erk-2 kinases (1 hr at RT). This antibody is biotinylated by standard procedures. The bound polyclonal antibody is then quantitated by successive incubations with Europium-streptavidin and Europium fluorescence enhancing reagent in the Wallac DELFIA instrument (time-resolved fluorescence). An increased fluorescent signal over background indicates a phosphorylation by D-SLAM or a molecule induced by D-SLAM.

### Example 22

### Method of Determining Alterations in the D-SLAM Gene

RNA isolated from entire families or individual patients presenting with a phenotype of interest (such as a disease) is be isolated. cDNA is then generated from these RNA samples using protocols known in the art. (See, Sambrook.) The cDNA is then used as a template for PCR, employing primers surrounding regions of interest in SEQ ID NO:1. Suggested PCR conditions consist of 35 cycles at 95 degree C. for 30 seconds; 60-120 seconds at 52-58 degree C.; and 60-120 seconds at 70 degree C., using buffer solutions described in Sidransky, D., et al., Science 252:706 (1991).

PCR products are then sequenced using primers labeled at their 5′ end with T4 polynucleotide kinase, employing SequiTherm Polymerase. (Epicentre Technologies). The intron-exon borders of selected exons of D-SLAM is also determined and genomic PCR products analyzed to confirm the results. PCR products harboring suspected mutations in D-SLAM is then cloned and sequenced to validate the results of the direct sequencing.

PCR products of D-SLAM are cloned into T-tailed vectors as described in Holton, T. A. and Graham, M. W., Nucleic Acids Research, 19:1156 (1991) and sequenced with T7 polymerase (United States Biochemical). Affected individuals are identified by mutations in D-SLAM not present in unaffected individuals.

Genomic rearrangements are also observed as a method of determining alterations in the D-SLAM gene. Genomic clones isolated according to Example 2 are nick-translated with digoxigenindeoxy-uridine 5′-triphosphate (Boehringer Manheim), and FISH performed as described in Johnson, Cg. et al., Methods Cell Biol. 35:73-99 (1991). Hybridization with the labeled probe is carried out using a vast excess of human cot-1 DNA for specific hybridization to the D-SLAM genomic locus.

Chromosomes are counterstained with 4,6-diamino-2-phenylidole and propidium iodide, producing a combination of C- and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters. (Johnson, Cv. et al., Genet. Anal. Tech. Appl., 8:75 (1991).) Image collection, analysis and chromosomal fractional length measurements are performed using the ISee Graphical Program System. (Inovision Corporation, Durham, N.C.) Chromosome alterations of the genomic region of D-SLAM (hybridized by the probe) are identified as insertions, deletions, and translocations. These D-SLAM alterations are used as a diagnostic marker for an associated disease.

### Example 23

### Method of Detecting Abnormal Levels of D-SLAM in a Biological Sample

D-SLAM polypeptides can be detected in a biological sample, and if an increased or decreased level of D-SLAM is detected, this polypeptide is a marker for a particular phenotype. Methods of detection are numerous, and thus, it is understood that one skilled in the art can modify the following assay to fit their particular needs.

For example, antibody-sandwich ELISAs are used to detect D-SLAM in a sample, preferably a biological sample. Wells of a microtiter plate are coated with specific antibodies to D-SLAM, at a final concentration of 0.2 to 10 ug/ml. The antibodies are either monoclonal or polyclonal and are produced by the method described in Example 11. The wells are blocked so that non-specific binding of D-SLAM to the well is reduced.

The coated wells are then incubated for >2 hours at RT with a sample containing D-SLAM. Preferably, serial dilutions of the sample should be used to validate results. The plates are then washed three times with deionized or distilled water to remove unbounded D-SLAM.

Next, 50 ul of specific antibody-alkaline phosphatase conjugate, at a concentration of 25-400 ng, is added and incubated for 2 hours at room temperature. The plates are again washed three times with deionized or distilled water to remove unbounded conjugate.

Add 75 ul of 4-methylumbelliferyl phosphate (MUP) or p-nitrophenyl phosphate (NPP) substrate solution to each well and incubate 1 hour at room temperature. Measure the reaction by a microtiter plate reader. Prepare a standard curve, using serial dilutions of a control sample, and plot D-SLAM polypeptide concentration on the X-axis (log scale) and fluorescence or absorbance of the Y-axis (linear scale). Interpolate the concentration of the D-SLAM in the sample using the standard curve.

### Example 24

### Formulating a Polypeptide

The D-SLAM composition will be formulated and dosed in a fashion consistent with good medical practice, taking into account the clinical condition of the individual patient (especially the side effects of treatment with the D-SLAM polypeptide alone), the site of delivery, the method of administration, the scheduling of administration, and other factors known to practitioners. The “effective amount” for purposes herein is thus determined by such considerations.

As a general proposition, the total pharmaceutically effective amount of D-SLAM administered parenterally per dose will be in the range of about 1 ug/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, D-SLAM is typically administered at a dose rate of about 1 ug/kg/hour to about 50 ug/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing D-SLAM are administered orally, rectally, parenterally, intracisternally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

D-SLAM is also suitably administered by sustained-release systems. Suitable examples of sustained-release compositions include semi-permeable polymer matrices in the form of shaped articles, e.g., films, or microcapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly(2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al.) or poly-D-(−)-3-hydroxybutyric acid (EP 133,988). Sustained-release compositions also include liposomally entrapped D-SLAM polypeptides. Liposomes containing the D-SLAM are prepared by methods known per se: DE 3,218,121; Epstein et al., Proc. Natl. Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal secreted polypeptide therapy.

For parenteral administration, in one embodiment, D-SLAM is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting D-SLAM uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

D-SLAM is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of polypeptide salts.

D-SLAM used for therapeutic administration can be sterile. Sterility is readily accomplished by filtration through sterile filtration membranes (e.g., 0.2 micron membranes). Therapeutic polypeptide compositions generally are placed into a container having a sterile access port, for example, an intravenous solution bag or vial having a stopper pierceable by a hypodermic injection needle.

D-SLAM polypeptides ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous D-SLAM polypeptide solution, and the resulting mixture is lyophilized. The infusion solution is prepared by reconstituting the lyophilized D-SLAM polypeptide using bacteriostatic Water-for-Injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, D-SLAM may be employed in conjunction with other therapeutic compounds.

### Example 25

### Method of Treating Decreased Levels of D-SLAM

The present invention relates to a method for treating an individual in need of a decreased level of D-SLAM activity in the body comprising, administering to such an individual a composition comprising a therapeutically effective amount of D-SLAM antagonist. Preferred antagonists for use in the present invention are D-SLAM-specific antibodies.

Moreover, it will be appreciated that conditions caused by a decrease in the standard or normal expression level of D-SLAM in an individual can be treated by administering D-SLAM, preferably in the secreted form. Thus, the invention also provides a method of treatment of an individual in need of an increased level of D-SLAM polypeptide comprising administering to such an individual a pharmaceutical composition comprising an amount of D-SLAM to increase the activity level of D-SLAM in such an individual.

For example, a patient with decreased levels of D-SLAM polypeptide receives a daily dose 0.1-100 ug/kg of the polypeptide for six consecutive days. Preferably, the polypeptide is in the secreted form. The exact details of the dosing scheme, based on administration and formulation, are provided in Example 24.

### Example 26

### Method of Treating Increased Levels of D-SLAM

The present invention also relates to a method for treating an individual in need of an increased level of D-SLAM activity in the body comprising administering to such an individual a composition comprising a therapeutically effective amount of D-SLAM or an agonist thereof.

Antisense technology is used to inhibit production of D-SLAM. This technology is one example of a method of decreasing levels of D-SLAM polypeptide, preferably a secreted form, due to a variety of etiologies, such as cancer.

For example, a patient diagnosed with abnormally increased levels of D-SLAM is administered intravenously antisense polynucleotides at 0.5, 1.0, 1.5, 2.0 and 3.0 mg/kg day for 21 days. This treatment is repeated after a 7-day rest period if the treatment was well tolerated. The formulation of the antisense polynucleotide is provided in Example 24.

### Example 27

### Method of Treatment Using Gene Therapy—Ex Vivo

One method of gene therapy transplants fibroblasts, which are capable of expressing D-SLAM polypeptides, onto a patient. Generally, fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin) is added. The flasks are then incubated at 37 degree C. for approximately one week.

At this time, fresh media is added and subsequently changed every several days. After an additional two weeks in culture, a monolayer of fibroblasts emerge. The monolayer is trypsinized and scaled into larger flasks.

pMV-7 (Kirschmeier, P. T. et al., DNA, 7:219-25 (1988)), flanked by the long terminal repeats of the Moloney murine sarcoma virus, is digested with EcoRI and HindIII and subsequently treated with calf intestinal phosphatase. The linear vector is fractionated on agarose gel and purified, using glass beads.

The cDNA encoding D-SLAM can be amplified using PCR primers which correspond to the 5′ and 3′ end sequences respectively as set forth in Example 1. Preferably, the 5′ primer contains an EcoRI site and the 3′ primer includes a HindIII site. Equal quantities of the Moloney murine sarcoma virus linear backbone and the amplified EcoRI and HindIII fragment are added together, in the presence of T4 DNA ligase. The resulting mixture is maintained under conditions appropriate for ligation of the two fragments. The ligation mixture is then used to transform bacteria HB101, which are then plated onto agar containing kanamycin for the purpose of confirming that the vector contains properly inserted D-SLAM.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the D-SLAM gene is then added to the media and the packaging cells transduced with the vector. The packaging cells now produce infectious viral particles containing the D-SLAM gene (the packaging cells are now referred to as producer cells).

Fresh media is added to the transduced producer cells, and subsequently, the media is harvested from a 10 cm plate of confluent producer cells. The spent media, containing the infectious viral particles, is filtered through a millipore filter to remove detached producer cells and this media is then used to infect fibroblast cells. Media is removed from a sub-confluent plate of fibroblasts and quickly replaced with the media from the producer cells. This media is removed and replaced with fresh media. If the titer of virus is high, then virtually all fibroblasts will be infected and no selection is required. If the titer is very low, then it is necessary to use a retroviral vector that has a selectable marker, such as neo or his. Once the fibroblasts have been efficiently infected, the fibroblasts are analyzed to determine whether D-SLAM protein is produced.

The engineered fibroblasts are then transplanted onto the host, either alone or after having been grown to confluence on cytodex 3 microcarrier beads.

### Example 28

### Method of Treatment Using Gene Therapy—In Vivo

Another aspect of the present invention is using in vivo gene therapy methods to treat disorders, diseases and conditions. The gene therapy method relates to the introduction of naked nucleic acid (DNA, RNA, and antisense DNA or RNA) D-SLAM sequences into an animal to increase or decrease the expression of the D-SLAM polypeptide. The D-SLAM polynucleotide may be operatively linked to a promoter or any other genetic elements necessary for the expression of the D-SLAM polypeptide by the target tissue. Such gene therapy and delivery techniques and methods are known in the art, see, for example, WO 90/11092, WO 98/11779; U.S. Pat. Nos. 5,693,622, 5,705,151, 5,580,859; Tabata H. et al. (1997) Cardiovasc. Res. 35(3):470-479, Chao J et al. (1997) Pharmacol. Res. 35(6):517-522, Wolff J. A. (1997) Neuromuscul. Disord. 7(5):314-318, Schwartz B. et al. (1996) Gene Ther. 3(5):405-411, Tsurumi Y. et al. (1996) Circulation 94(12):3281-3290 (incorporated herein by reference).

The D-SLAM polynucleotide constructs may be delivered by any method that delivers injectable materials to the cells of an animal, such as, injection into the interstitial space of tissues (heart, muscle, skin, lung, liver, intestine and the like). The D-SLAM polynucleotide constructs can be delivered in a pharmaceutically acceptable liquid or aqueous carrier.

The term “naked” polynucleotide, DNA or RNA, refers to sequences that are free from any delivery vehicle that acts to assist, promote, or facilitate entry into the cell, including viral sequences, viral particles, liposome formulations, lipofectin or precipitating agents and the like. However, the D-SLAM polynucleotides may also be delivered in liposome formulations (such as those taught in Felgner P. L. et al. (1995) Ann. NY Acad. Sci. 772:126-139 and Abdallah B. et al. (1995) Biol. Cell 85(1):1-7) which can be prepared by methods well known to those skilled in the art.

The D-SLAM polynucleotide vector constructs used in the gene therapy method are preferably constructs that will not integrate into the host genome nor will they contain sequences that allow for replication. Any strong promoter known to those skilled in the art can be used for driving the expression of DNA. Unlike other gene therapies techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The D-SLAM polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked D-SLAM polynucleotide injection, an effective dosage amount of DNA or RNA will be in the range of from about 0.05 g/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration. The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked D-SLAM polynucleotide constructs can be delivered to arteries during angioplasty by the catheter used in the procedure.

The dose response effects of injected D-SLAM polynucleotide in muscle in vivo is determined as follows. Suitable D-SLAM template DNA for production of mRNA coding for D-SLAM polypeptide is prepared in accordance with a standard recombinant DNA methodology. The template DNA, which may be either circular or linear, is either used as naked DNA or complexed with liposomes. The quadriceps muscles of mice are then injected with various amounts of the template DNA.

Five to six week old female and male Balb/C mice are anesthetized by intraperitoneal injection with 0.3 ml of 2.5% Avertin. A 1.5 cm incision is made on the anterior thigh, and the quadriceps muscle is directly visualized. The D-SLAM template DNA is injected in 0.1 ml of carrier in a 1 cc syringe through a 27 gauge needle over one minute, approximately 0.5 cm from the distal insertion site of the muscle into the knee and about 0.2 cm deep. A suture is placed over the injection site for future localization, and the skin is closed with stainless steel clips.

After an appropriate incubation time (e.g., 7 days) muscle extracts are prepared by excising the entire quadriceps. Every fifth 15 um cross-section of the individual quadriceps muscles is histochemically stained for D-SLAM protein expression. A time course for D-SLAM protein expression may be done in a similar fashion except that quadriceps from different mice are harvested at different times. Persistence of D-SLAM DNA in muscle following injection may be determined by Southern blot analysis after preparing total cellular DNA and HIRT supernatants from injected and control mice. The results of the above experimentation in mice can be use to extrapolate proper dosages and other treatment parameters in humans and other animals using D-SLAM naked DNA.

### Example 29

### D-SLAM Transgenic Animals

The D-SLAM polypeptides can also be expressed in transgenic animals. Animals of any species, including, but not limited to, mice, rats, rabbits, hamsters, guinea pigs, pigs, micro-pigs, goats, sheep, cows and non-human primates, e.g., baboons, monkeys, and chimpanzees may be used to generate transgenic animals. In a specific embodiment, techniques described herein or otherwise known in the art, are used to express polypeptides of the invention in humans, as part of a gene therapy protocol.

Any technique known in the art may be used to introduce the transgene (i.e., polynucleotides of the invention) into animals to produce the founder lines of transgenic animals. Such techniques include, but are not limited to, pronuclear microinjection (Paterson et al., Appl. Microbiol. Biotechnol. 40:691-698 (1994); Carver et al., Biotechnology (NY) 11:1263-1270 (1993); Wright et al., Biotechnology (NY) 9:830-834 (1991); and Hoppe et al., U.S. Pat. No. 4,873,191 (1989)); retrovirus mediated gene transfer into germ lines (Van der Putten et al., Proc. Natl. Acad. Sci., USA 82:6148-6152 (1985)), blastocysts or embryos; gene targeting in embryonic stem cells (Thompson et al., Cell 56:313-321 (1989)); electroporation of cells or embryos (Lo, 1983, Mol Cell. Biol. 3:1803-1814 (1983)); introduction of the polynucleotides of the invention using a gene gun (see, e.g., Ulmer et al., Science 259:1745 (1993); introducing nucleic acid constructs into embryonic pleuripotent stem cells and transferring the stem cells back into the blastocyst; and sperm-mediated gene transfer (Lavitrano et al., Cell 57:717-723 (1989); etc. For a review of such techniques, see Gordon, “Transgenic Animals,” Intl. Rev. Cytol. 115:171-229 (1989), which is incorporated by reference herein in its entirety.

Any technique known in the art may be used to produce transgenic clones containing polynucleotides of the invention, for example, nuclear transfer into enucleated oocytes of nuclei from cultured embryonic, fetal, or adult cells induced to quiescence (Campell et al., Nature 380:64-66 (1996); Wilmut et al., Nature 385:810-813 (1997)).

The present invention provides for transgenic animals that carry the transgene in all their cells, as well as animals which carry the transgene in some, but not all their cells, i.e., mosaic animals or chimeric. The transgene may be integrated as a single transgene or as multiple copies such as in concatamers, e.g., head-to-head tandems or head-to-tail tandems. The transgene may also be selectively introduced into and activated in a particular cell type by following, for example, the teaching of Lasko et al. (Lasko et al., Proc. Natl. Acad. Sci. USA 89:6232-6236 (1992)). The regulatory sequences required for such a cell-type specific activation will depend upon the particular cell type of interest, and will be apparent to those of skill in the art. When it is desired that the polynucleotide transgene be integrated into the chromosomal site of the endogenous gene, gene targeting is preferred.

Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al. (Gu et al., Science 265:103-106 (1994)). The regulatory sequences required for such a cell-type specific inactivation will depend upon the particular cell type of interest, and will be apparent to those of skill in the art.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR (rt-PCR). Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic animals of the invention have uses which include, but are not limited to, animal model systems useful in elaborating the biological function of D-SLAM polypeptides, studying conditions and/or disorders associated with aberrant D-SLAM expression, and in screening for compounds effective in ameliorating such conditions and/or disorders.

### Example 30

### D-SLAM Knock-Out Animals

Endogenous D-SLAM gene expression can also be reduced by inactivating or “knocking out” the D-SLAM gene and/or its promoter using targeted homologous recombination. (E.g., see Smithies et al., Nature 317:230-234 (1985); Thomas & Capecchi, Cell 51:503-512 (1987); Thompson et al., Cell 5:313-321 (1989); each of which is incorporated by reference herein in its entirety). For example, a mutant, non-functional polynucleotide of the invention (or a completely unrelated DNA sequence) flanked by DNA homologous to the endogenous polynucleotide sequence (either the coding regions or regulatory regions of the gene) can be used, with or without a selectable marker and/or a negative selectable marker, to transfect cells that express polypeptides of the invention in vivo. In another embodiment, techniques known in the art are used to generate knockouts in cells that contain, but do not express the gene of interest. Insertion of the DNA construct, via targeted homologous recombination, results in inactivation of the targeted gene. Such approaches are particularly suited in research and agricultural fields where modifications to embryonic stem cells can be used to generate animal offspring with an inactive targeted gene (e.g., see Thomas & Capecchi 1987 and Thompson 1989, supra). However this approach can be routinely adapted for use in humans provided the recombinant DNA constructs are directly administered or targeted to the required site in vivo using appropriate viral vectors that will be apparent to those of skill in the art.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from the patient (i.e., animal, including human) or an MHC compatible donor and can include, but are not limited to fibroblasts, bone marrow cells, blood cells (e.g., lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and preferably vectors that integrate the transgene into the cell genome) or transfection procedures, including, but not limited to, the use of plasmids, cosmids, YACs, naked DNA, electroporation, liposomes, etc. The coding sequence of the polypeptides of the invention can be placed under the control of a strong constitutive or inducible promoter or promoter/enhancer to achieve expression, and preferably secretion, of the D-SLAM polypeptides. The engineered cells which express and preferably secrete the polypeptides of the invention can be introduced into the patient systemically, e.g., in the circulation, or intraperitoneally.

Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. (See, for example, Anderson et al. U.S. Pat. No. 5,399,349; and Mulligan & Wilson, U.S. Pat. No. 5,460,959 each of which is incorporated by reference herein in its entirety).

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Knock-out animals of the invention have uses which include, but are not limited to, animal model systems useful in elaborating the biological function of D-SLAM polypeptides, studying conditions and/or disorders associated with aberrant D-SLAM expression, and in screening for compounds effective in ameliorating such conditions and/or disorders.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference. Moreover, the sequence listing is herein incorporated by reference.

TABLE 1A

| | | Garni . . . | Chou- . . . | Garni . . . | Chou- . . . | Garni . . . | Chou- . . . | Garni . . . | Kyte- . . . |
|---|---|---|---|---|---|---|---|---|---|
| Res | Pos | Alpha | Alpha | Beta | Beta | Turn | Turn | Coil | Hydro . . . |
| Met | 1 | — | — | B | — | — | — | — | −0.21 |
| Val | 2 | — | — | B | — | — | — | — | −0.63 |
| Met | 3 | — | — | B | — | — | — | — | −0.53 |
| Arg | 4 | — | — | B | — | — | T | — | −0.44 |
| Pro | 5 | — | — | B | — | — | T | — | −0.87 |
| Leu | 6 | — | — | — | — | T | T | — | −1.08 |
| Trp | 7 | — | — | B | — | — | T | — | −1.03 |
| Ser | 8 | — | A | B | — | — | — | — | −0.72 |
| Leu | 9 | — | A | B | — | — | — | — | −0.83 |
| Leu | 10 | — | A | B | — | — | — | — | −1.21 |
| Leu | 11 | — | A | B | — | — | — | — | −1.21 |
| Trp | 12 | — | A | B | — | — | — | — | −1.73 |
| Glu | 13 | — | A | B | — | — | — | — | −1.64 |
| Ala | 14 | — | A | B | — | — | — | — | −1.72 |
| Leu | 15 | — | A | B | — | — | — | — | −1.22 |
| Leu | 16 | — | A | B | — | — | — | — | −1.27 |
| Pro | 17 | — | A | B | — | — | — | — | −1.29 |
| Ile | 18 | — | — | B | B | — | — | — | −1.63 |
| Thr | 19 | — | — | B | B | — | — | — | −1.63 |
| Val | 20 | — | — | B | B | — | — | — | −0.82 |
| Thr | 21 | — | — | B | B | — | — | — | −0.87 |
| Gly | 22 | — | — | B | B | — | — | — | −1.47 |
| Ala | 23 | — | — | B | B | — | — | — | −0.88 |
| Gln | 24 | — | — | B | B | — | — | — | −0.52 |
| Val | 25 | — | — | B | B | — | — | — | −0.52 |
| Leu | 26 | — | — | B | B | — | — | — | −0.56 |
| Ser | 27 | — | — | B | B | — | — | — | −0.56 |
| Lys | 28 | — | — | B | B | — | — | — | −0.27 |
| Val | 29 | — | — | B | — | — | T | — | −1.12 |
| Gly | 30 | — | — | — | — | T | T | — | −1.08 |
| Gly | 31 | — | — | B | — | — | T | — | −1.08 |
| Ser | 32 | — | — | B | — | — | T | — | −1.63 |
| Val | 33 | — | A | B | — | — | — | — | −2.27 |
| Leu | 34 | — | A | B | — | — | — | — | −2.00 |

| | | Eisen . . . | Eisen . . . | Karpl . . . | James . . . | Emini |
|---|---|---|---|---|---|---|
| Res | Pos | Alpha | Beta | Flexi . . . | Antig . . . | Surfa . . . |
| Met | 1 | — | — | — | −0.10 | 0.53 |
| Val | 2 | — | — | — | −0.10 | 0.56 |
| Met | 3 | — | — | — | −0.40 | 0.42 |
| Arg | 4 | — | — | — | −0.20 | 0.44 |
| Pro | 5 | — | — | — | −0.20 | 0.80 |
| Leu | 6 | — | — | — | 0.20 | 0.67 |
| Trp | 7 | — | — | — | −0.20 | 0.28 |
| Ser | 8 | — | — | — | −0.60 | 0.15 |
| Leu | 9 | — | — | — | −0.60 | 0.19 |
| Leu | 10 | — | — | — | −0.60 | 0.31 |
| Leu | 11 | — | — | — | −0.60 | 0.24 |
| Trp | 12 | — | — | — | −0.60 | 0.24 |
| Glu | 13 | — | — | — | −0.60 | 0.24 |
| Ala | 14 | — | — | — | −0.60 | 0.44 |
| Leu | 15 | — | — | — | −0.60 | 0.30 |
| Leu | 16 | — | — | — | −0.60 | 0.25 |
| Pro | 17 | — | — | — | −0.60 | 0.18 |
| Ile | 18 | — | — | — | −0.60 | 0.32 |
| Thr | 19 | — | — | — | −0.60 | 0.38 |
| Val | 20 | — | — | — | −0.60 | 0.25 |
| Thr | 21 | — | — | F | −0.45 | 0.61 |
| Gly | 22 | — | — | F | −0.45 | 0.32 |
| Ala | 23 | — | — | — | −0.60 | 0.35 |
| Gln | 24 | — | — | — | −0.60 | 0.33 |
| Val | 25 | — | — | — | −0.30 | 0.66 |
| Leu | 26 | — | — | — | −0.30 | 0.48 |
| Ser | 27 | — | — | F | −0.10 | 0.28 |
| Lys | 28 | — | — | F | −0.05 | 0.37 |
| Val | 29 | — | — | F | 1.00 | 0.60 |
| Gly | 30 | — | — | F | 1.45 | 0.33 |
| Gly | 31 | — | — | F | 0.50 | 0.14 |
| Ser | 32 | — | — | F | 0.15 | 0.15 |
| Val | 33 | — | — | — | −0.45 | 0.11 |
| Leu | 34 | — | — | — | −0.50 | 0.12 |

TABLE 1B

| | | Garni . . . | Chou- . . . | Garni . . . | Chou- . . . | Garni . . . | Chou- . . . | Garni . . . | Kyte- . . . |
|---|---|---|---|---|---|---|---|---|---|
| Res | Pos | Alpha | Alpha | Beta | Beta | Turn | Turn | Coil | Hydro . . . |
| Leu | 35 | — | A | B | — | — | — | — | −1.51 |
| Val | 36 | — | A | B | — | — | — | — | −1.41 |
| Ala | 37 | — | A | B | — | — | — | — | −1.32 |
| Ala | 38 | — | A | B | — | — | — | — | −0.81 |
| Arg | 39 | — | A | — | — | — | — | C | −0.70 |
| Pro | 40 | — | — | — | — | — | T | C | 0.11 |
| Pro | 41 | — | — | — | — | T | T | — | 0.11 |
| Gly | 42 | — | — | — | — | T | T | — | 0.81 |
| Phe | 43 | — | — | B | — | — | T | — | 1.40 |
| Gln | 44 | — | A | B | B | — | — | — | 0.70 |
| Val | 45 | — | A | B | B | — | — | — | 0.02 |
| Arg | 46 | — | A | B | B | — | — | — | −0.06 |
| Glu | 47 | — | A | B | — | — | — | — | 0.40 |
| Ala | 48 | — | A | B | — | — | — | — | 0.80 |
| Ile | 49 | — | A | — | — | T | — | — | −0.01 |
| Trp | 50 | — | A | — | — | T | — | — | 0.56 |
| Arg | 51 | — | A | — | — | — | — | C | 0.23 |
| Ser | 52 | — | — | — | — | — | — | C | −0.07 |
| Leu | 53 | — | — | — | — | — | — | C | 0.52 |
| Trp | 54 | — | — | — | — | — | T | C | 1.41 |
| Pro | 55 | — | — | — | — | — | T | C | 0.89 |
| Ser | 56 | — | — | — | — | — | T | C | −0.03 |
| Glu | 57 | — | — | — | — | — | T | C | −0.32 |
| Glu | 58 | — | A | B | — | — | — | — | 0.18 |
| Leu | 59 | — | A | B | — | — | — | — | −0.23 |
| Leu | 60 | — | A | B | — | — | — | — | −0.72 |
| Ala | 61 | — | A | B | — | — | — | — | −0.31 |
| Thr | 62 | — | A | B | — | — | — | — | −0.66 |
| Phe | 63 | — | A | B | — | — | — | — | −0.96 |
| Phe | 64 | — | — | B | — | — | T | — | −0.96 |
| Arg | 65 | — | — | — | — | — | T | C | −0.14 |
| Gly | 66 | — | — | — | — | — | T | C | 0.13 |
| Ser | 67 | — | — | — | — | — | T | C | −0.37 |
| Leu | 68 | — | — | — | — | — | — | C | 0.09 |

| | | Eisen . . . | Eisen . . . | Karpl . . . | James . . . | Emini |
|---|---|---|---|---|---|---|
| Res | Pos | Alpha | Beta | Flexi . . . | Antig . . . | Surfa . . . |
| Leu | 35 | — | — | — | −0.55 | 0.09 |
| Val | 36 | — | — | — | −0.60 | 0.23 |
| Ala | 37 | — | — | — | −0.60 | 0.43 |
| Ala | 38 | — | — | — | −0.30 | 0.81 |
| Arg | 39 | — | — | — | 0.65 | 1.08 |
| Pro | 40 | — | — | F | 0.45 | 0.93 |
| Pro | 41 | — | — | F | 1.40 | 1.59 |
| Gly | 42 | — | — | F | 0.65 | 0.60 |
| Phe | 43 | — | — | — | 0.10 | 0.76 |
| Gln | 44 | — | — | — | 0.30 | 0.86 |
| Val | 45 | — | — | — | 0.30 | 0.87 |
| Arg | 46 | — | — | — | −0.30 | 0.71 |
| Glu | 47 | — | — | — | −0.30 | 0.43 |
| Ala | 48 | — | — | — | 0.45 | 1.13 |
| Ile | 49 | — | — | — | 1.00 | 0.78 |
| Trp | 50 | — | — | — | 0.10 | 0.37 |
| Arg | 51 | — | — | — | −0.16 | 0.38 |
| Ser | 52 | — | — | — | 0.28 | 0.85 |
| Leu | 53 | — | — | F | 0.82 | 1.08 |
| Trp | 54 | — | — | F | 2.01 | 0.96 |
| Pro | 55 | — | — | F | 2.40 | 1.24 |
| Ser | 56 | — | — | F | 1.56 | 1.24 |
| Glu | 57 | — | — | F | 1.17 | 0.97 |
| Glu | 58 | — | — | F | 0.93 | 0.63 |
| Leu | 59 | — | — | — | 0.54 | 0.68 |
| Leu | 60 | — | — | — | −0.30 | 0.34 |
| Ala | 61 | — | — | — | −0.60 | 0.17 |
| Thr | 62 | — | — | — | −0.60 | 0.40 |
| Phe | 63 | — | — | — | −0.60 | 0.49 |
| Phe | 64 | — | — | — | 0.27 | 0.64 |
| Arg | 65 | — | — | F | 0.49 | 0.37 |
| Gly | 66 | — | — | F | 0.96 | 0.74 |
| Ser | 67 | — | — | F | 1.88 | 1.23 |
| Leu | 68 | — | — | F | 1.70 | 0.52 |

TABLE 1C

| | | Garni . . . | Chou- . . . | Garni . . . | Chou- . . . | Garni . . . | Chou- . . . | Garni . . . | Kyte- . . . |
|---|---|---|---|---|---|---|---|---|---|
| Res | Pos | Alpha | Alpha | Beta | Beta | Turn | Turn | Coil | Hydro . . . |
| Glu | 69 | — | — | — | — | — | — | C | 0.76 |
| Thr | 70 | — | — | B | — | — | — | — | 0.34 |
| Leu | 71 | — | — | B | — | — | — | — | 0.80 |
| Tyr | 72 | — | — | B | — | — | — | — | 0.40 |
| His | 73 | — | — | B | — | — | — | — | 0.40 |
| Ser | 74 | — | — | B | — | — | — | — | 0.06 |
| Arg | 75 | — | — | B | — | — | — | — | 0.48 |
| Phe | 76 | — | — | — | — | T | — | — | 0.70 |
| Leu | 77 | — | — | B | — | — | — | — | 0.94 |
| Gly | 78 | — | — | B | — | — | — | — | 0.17 |
| Arg | 79 | — | — | B | — | — | — | — | 0.43 |
| Ala | 80 | — | — | B | — | — | — | — | 0.02 |
| Gln | 81 | — | — | B | — | — | — | — | 0.72 |
| Leu | 82 | — | — | B | — | — | — | — | 0.72 |
| His | 83 | — | — | — | — | — | T | C | 0.77 |
| Ser | 84 | — | — | — | — | — | T | C | −0.16 |
| Asn | 85 | — | — | — | — | — | T | C | 0.43 |
| Leu | 86 | — | — | B | — | — | T | — | −0.38 |
| Ser | 87 | — | A | B | — | — | — | — | 0.09 |
| Leu | 88 | — | A | B | — | — | — | — | −0.09 |
| Glu | 89 | — | A | B | — | — | — | — | −0.60 |
| Leu | 90 | — | A | B | — | — | — | — | −0.60 |
| Gly | 91 | — | A | B | — | — | — | — | −0.09 |
| Pro | 92 | — | — | B | — | — | — | — | −0.13 |
| Leu | 93 | — | — | B | — | — | — | — | 0.68 |
| Glu | 94 | — | — | B | — | — | — | — | 0.38 |
| Ser | 95 | — | — | B | — | T | — | — | 0.84 |
| Gly | 96 | — | — | — | — | T | — | — | 1.19 |
| Asp | 97 | — | — | — | — | T | T | — | 0.70 |
| Ser | 98 | — | — | — | — | — | T | C | 1.21 |
| Gly | 99 | — | — | — | — | — | T | C | 0.36 |
| Asn | 100 | — | — | — | — | — | T | C | −0.16 |
| Phe | 101 | — | — | B | B | — | — | — | −0.41 |
| Ser | 102 | — | — | B | B | — | — | — | −1.27 |

| | | Eisen . . . | Eisen . . . | Karpl . . . | James . . . | Emini |
|---|---|---|---|---|---|---|
| Res | Pos | Alpha | Beta | Flexi . . . | Antig . . . | Surfa . . . |
| Glu | 69 | — | — | F | 0.63 | 0.82 |
| Thr | 70 | — | — | — | 0.11 | 0.83 |
| Leu | 71 | — | — | — | 0.09 | 1.35 |
| Tyr | 72 | — | — | — | 0.82 | 1.53 |
| His | 73 | — | — | — | −0.40 | 0.92 |
| Ser | 74 | — | — | — | −0.40 | 0.92 |
| Arg | 75 | — | — | — | −0.40 | 0.58 |
| Phe | 76 | — | — | — | 0.90 | 0.83 |
| Leu | 77 | — | — | — | 0.50 | 0.63 |
| Gly | 78 | — | — | — | 0.50 | 0.56 |
| Arg | 79 | — | — | — | −0.40 | 0.53 |
| Ala | 80 | — | — | — | −0.10 | 0.87 |
| Gln | 81 | — | — | — | 0.65 | 1.18 |
| Leu | 82 | — | — | — | 0.50 | 0.97 |
| His | 83 | — | — | — | 0.00 | 0.79 |
| Ser | 84 | — | — | — | 0.00 | 0.61 |
| Asn | 85 | — | — | — | 0.00 | 0.61 |
| Leu | 86 | — | — | — | 0.10 | 0.78 |
| Ser | 87 | — | — | — | −0.30 | 0.48 |
| Leu | 88 | — | — | — | −0.30 | 0.30 |
| Glu | 89 | — | — | — | −0.30 | 0.55 |
| Leu | 90 | — | — | — | −0.30 | 0.34 |
| Gly | 91 | — | — | F | 0.45 | 0.72 |
| Pro | 92 | — | — | F | 0.65 | 0.55 |
| Leu | 93 | — | — | F | 0.39 | 0.66 |
| Glu | 94 | — | — | F | 1.78 | 1.12 |
| Ser | 95 | — | — | F | 2.37 | 0.97 |
| Gly | 96 | — | — | F | 2.86 | 1.17 |
| Asp | 97 | — | — | F | 3.40 | 1.08 |
| Ser | 98 | — | — | F | 2.41 | 0.70 |
| Gly | 99 | — | — | F | 2.07 | 0.95 |
| Asn | 100 | — | — | F | 1.13 | 0.42 |
| Phe | 101 | — | — | — | −0.26 | 0.26 |
| Ser | 102 | — | — | — | −0.60 | 0.26 |

TABLE 1D

| | | Garni . . . | Chou- . . . | Garni . . . | Chou- . . . | Garni . . . | Chou- . . . | Garni . . . | Kyte- . . . |
|---|---|---|---|---|---|---|---|---|---|
| Res | Pos | Alpha | Alpha | Beta | Beta | Turn | Turn | Coil | Hydro . . . |
| Val | 103 | — | — | B | B | — | — | — | −0.97 |
| Leu | 104 | — | — | B | B | — | — | — | −0.93 |
| Mel | 105 | — | — | B | B | — | — | — | −0.82 |
| Val | 106 | — | — | B | B | — | — | — | −0.47 |
| Asp | 107 | — | — | B | — | — | T | — | −0.17 |
| Thr | 108 | — | — | — | — | T | T | — | 0.48 |
| Arg | 109 | — | — | — | — | T | T | — | 1.00 |
| Gly | 110 | — | — | — | — | — | T | C | 1.29 |
| Gln | 111 | — | — | — | — | — | T | C | 2.14 |
| Pro | 112 | — | — | — | — | — | T | C | 1.83 |
| Trp | 113 | — | — | — | — | T | T | — | 1.33 |
| Thr | 114 | — | — | B | — | — | T | — | 1.22 |
| Gln | 115 | — | — | B | B | — | — | — | 0.76 |
| Thr | 116 | — | — | B | B | — | — | — | 0.80 |
| Leu | 117 | — | — | B | B | — | — | — | 0.16 |
| Gln | 118 | — | — | B | B | — | — | — | 0.20 |
| Leu | 119 | — | — | B | B | — | — | — | 0.51 |
| Lys | 120 | — | — | B | B | — | — | — | −0.08 |
| Val | 121 | — | — | B | B | — | — | — | −0.62 |
| Tyr | 122 | — | — | B | B | — | — | — | −0.02 |
| Asp | 123 | — | — | B | B | — | — | — | 0.09 |
| Ala | 124 | — | — | B | — | — | — | — | 0.69 |
| Val | 125 | — | — | B | — | — | T | — | −0.21 |
| Pro | 126 | — | — | B | — | — | T | — | −0.21 |
| Arg | 127 | — | — | B | — | — | T | — | 0.03 |
| Pro | 128 | — | — | B | — | — | T | — | −0.82 |
| Val | 129 | — | — | B | B | — | — | — | −0.93 |
| Val | 130 | — | — | B | B | — | — | — | −0.97 |
| Gln | 131 | — | — | B | B | — | — | — | −1.34 |
| Val | 132 | — | — | B | B | — | — | — | −2.31 |
| Phe | 133 | — | — | B | B | — | — | — | −2.10 |
| Ile | 134 | — | — | B | B | — | — | — | −1.13 |
| Ala | 135 | — | — | B | B | — | — | — | −0.28 |
| Val | 136 | — | — | B | B | — | — | — | −0.87 |

| | | Eisen . . . | Eisen . . . | Karpl . . . | James . . . | Emini |
|---|---|---|---|---|---|---|
| Res | Pos | Alpha | Beta | Flexi . . . | Antig . . . | Surfa . . . |
| Val | 103 | — | — | — | −0.60 | 0.12 |
| Leu | 104 | — | — | — | −0.60 | 0.23 |
| Mel | 105 | — | — | — | −0.26 | 0.25 |
| Val | 106 | — | — | — | 0.38 | 0.66 |
| Asp | 107 | — | — | F | 1.87 | 0.79 |
| Thr | 108 | — | — | F | 2.76 | 1.38 |
| Arg | 109 | — | — | F | 3.40 | 2.87 |
| Gly | 110 | — | — | F | 2.56 | 1.81 |
| Gln | 111 | — | — | F | 1.62 | 1.81 |
| Pro | 112 | — | — | F | 1.28 | 1.60 |
| Trp | 113 | — | — | F | 0.84 | 2.33 |
| Thr | 114 | — | — | F | 0.10 | 1.11 |
| Gln | 115 | — | — | F | −0.30 | 1.24 |
| Thr | 116 | — | — | F | −0.45 | 0.97 |
| Leu | 117 | — | — | F | 0.00 | 1.35 |
| Gln | 118 | — | — | — | −0.30 | 0.58 |
| Leu | 119 | — | — | — | −0.60 | 0.63 |
| Lys | 120 | — | — | — | −0.15 | 1.27 |
| Val | 121 | — | — | — | 0.30 | 0.74 |
| Tyr | 122 | — | — | — | −0.30 | 0.67 |
| Asp | 123 | — | — | — | 0.30 | 0.52 |
| Ala | 124 | — | — | — | 0.65 | 1.36 |
| Val | 125 | — | — | — | 0.85 | 1.34 |
| Pro | 126 | — | — | F | 0.85 | 0.60 |
| Arg | 127 | — | — | F | 0.25 | 0.44 |
| Pro | 128 | — | — | F | 0.40 | 1.02 |
| Val | 129 | — | — | — | −0.30 | 0.49 |
| Val | 130 | — | — | — | −0.60 | 0.22 |
| Gln | 131 | — | — | — | −0.60 | 0.10 |
| Val | 132 | — | — | — | −0.60 | 0.13 |
| Phe | 133 | — | — | — | −0.60 | 0.13 |
| Ile | 134 | — | — | — | −0.60 | 0.13 |
| Ala | 135 | — | — | — | −0.30 | 0.35 |
| Val | 136 | — | — | — | 0.30 | 0.68 |

TABLE 1E

| | | Garni . . . | Chou- . . . | Garni . . . | Chou- . . . | Garni . . . | Chou- . . . | Garni . . . | Kyte- . . . |
|---|---|---|---|---|---|---|---|---|---|
| Res | Pos | Alpha | Alpha | Beta | Beta | Turn | Turn | Coil | Hydro . . . |
| Glu | 137 | | — | B | B | — | — | — | −0.01 |
| Arg | 138 | — | — | — | — | T | — | — | 0.48 |
| Asp | 139 | — | — | — | — | T | — | — | 1.07 |
| Ala | 140 | — | — | — | — | T | — | — | 1.70 |
| Gln | 141 | — | — | — | — | — | T | C | 2.24 |
| Pro | 142 | — | — | — | — | T | T | — | 1.58 |
| Ser | 143 | — | — | — | — | T | T | — | 1.47 |
| Lys | 144 | — | — | — | — | T | T | — | 0.61 |
| Thr | 145 | — | — | B | B | — | — | — | 0.50 |
| Cys | 146 | — | — | B | B | — | — | — | −0.31 |
| Gln | 147 | — | — | B | B | — | — | — | −0.40 |
| Val | 148 | — | — | B | B | — | — | — | −0.77 |
| Phe | 149 | — | — | B | B | — | — | — | −1.10 |
| Leu | 150 | — | — | B | B | — | — | — | −1.38 |
| Ser | 151 | — | — | B | B | — | — | — | −0.92 |
| Cys | 152 | — | — | — | B | T | — | — | −0.92 |
| Trp | 153 | — | — | — | B | T | — | — | −0.96 |
| Ala | 154 | — | — | — | — | — | T | C | −0.56 |
| Pro | 155 | — | — | — | — | — | T | C | 0.26 |
| Asn | 156 | — | — | — | — | — | T | C | −0.33 |
| Ile | 157 | — | — | B | — | — | T | — | 0.02 |
| Ser | 158 | — | — | B | B | — | — | — | 0.07 |
| Glu | 159 | — | — | B | B | — | — | — | 0.36 |
| Ile | 160 | — | — | B | B | — | — | — | 0.28 |
| Thr | 161 | — | — | B | B | — | — | — | 0.39 |
| Tyr | 162 | — | — | — | B | T | — | — | 1.39 |
| Ser | 163 | — | — | — | B | — | — | C | 1.69 |
| Trp | 164 | — | — | — | B | — | — | C | 1.30 |
| Arg | 165 | — | — | — | B | — | — | C | 1.96 |
| Arg | 166 | — | — | | B | T | — | — | 1.67 |
| Glu | 167 | — | — | — | B | T | — | — | 1.91 |
| Thr | 168 | — | — | — | — | T | — | — | 1.51 |
| Thr | 169 | — | — | — | — | — | — | C | 1.46 |
| Met | 170 | — | — | — | — | — | — | C | 0.74 |

| | | Eisen . . . | Eisen . . . | Karpl . . . | James . . . | Emini |
|---|---|---|---|---|---|---|
| Res | Pos | Alpha | Beta | Flexi . . . | Antig . . . | Surfa . . . |
| Glu | 137 | — | — | — | 0.60 | 0.98 |
| Arg | 138 | — | — | F | 1.84 | 1.69 |
| Asp | 139 | — | — | F | 2.18 | 3.51 |
| Ala | 140 | — | — | F | 2.52 | 2.72 |
| Gln | 141 | — | — | F | 2.86 | 2.78 |
| Pro | 142 | — | — | F | 3.40 | 2.40 |
| Ser | 143 | — | — | F | 2.76 | 1.27 |
| Lys | 144 | — | — | F | 2.42 | 1.27 |
| Thr | 145 | — | — | F | 1.13 | 0.61 |
| Cys | 146 | — | — | — | 0.04 | 0.39 |
| Gln | 147 | — | — | — | −0.60 | 0.16 |
| Val | 148 | — | — | — | −0.60 | 0.15 |
| Phe | 149 | — | — | — | −0.60 | 0.15 |
| Leu | 150 | — | — | — | −0.60 | 0.09 |
| Ser | 151 | — | — | — | −0.60 | 0.12 |
| Cys | 152 | — | — | — | −0.20 | 0.22 |
| Trp | 153 | — | — | — | −0.20 | 0.43 |
| Ala | 154 | — | — | — | 0.00 | 0.23 |
| Pro | 155 | — | — | — | 0.00 | 0.57 |
| Asn | 156 | — | — | F | 0.45 | 0.94 |
| Ile | 157 | — | — | F | 0.25 | 0.65 |
| Ser | 158 | — | — | F | −0.15 | 0.61 |
| Glu | 159 | — | — | F | −0.45 | 0.59 |
| Ile | 160 | — | — | — | −0.15 | 1.13 |
| Thr | 161 | — | — | | −0.60 | 0.89 |
| Tyr | 162 | — | — | — | 0.25 | 1.00 |
| Ser | 163 | — | — | — | 0.05 | 2.80 |
| Trp | 164 | — | — | — | 0.95 | 3.36 |
| Arg | 165 | — | — | F | 1.40 | 3.10 |
| Arg | 166 | — | — | F | 2.20 | 3.34 |
| Glu | 167 | — | — | F | 2.20 | 3.14 |
| Thr | 168 | — | — | F | 3.00 | 2.68 |
| Thr | 169 | — | — | F | 2.50 | 1.18 |
| Met | 170 | — | — | — | 1.60 | 0.68 |

TABLE 1F

| | | Garni . . . | Chou- . . . | Garni . . . | Chou- . . . | Garni . . . | Chou- . . . | Garni . . . | Kyte- . . . |
|---|---|---|---|---|---|---|---|---|---|
| Res | Pos | Alpha | Alpha | Beta | Beta | Turn | Turn | Coil | Hydro . . . |
| Asp | 171 | — | — | | — | T | — | — | 0.63 |
| Phe | 172 | — | | B | — | — | — | C | 0.42 |
| Gly | 173 | — | — | — | — | — | — | C | 0.70 |
| Met | 174 | — | — | | — | — | — | C | 0.71 |
| Glu | 175 | | — | — | — | — | T | C | 0.50 |
| Pro | 176 | — | — | — | — | — | T | C | −0.20 |
| His | 177 | — | — | — | — | T | T | — | 0.19 |
| Ser | 178 | — | — | — | — | — | T | C | 0.53 |
| Leu | 179 | — | — | B | — | — | — | — | 0.79 |
| Phe | 180 | — | — | B | — | — | — | — | 0.79 |
| Thr | 181 | — | — | B | — | — | T | — | 0.14 |
| Asp | 182 | — | — | — | — | T | T | — | −0.63 |
| Gly | 183 | — | — | B | — | — | T | — | −0.63 |
| Gln | 184 | — | — | B | — | — | T | — | −0.71 |
| Val | 185 | — | — | B | B | — | — | — | −0.31 |
| Leu | 186 | — | — | B | B | — | — | — | −0.81 |
| Ser | 187 | — | — | B | B | — | — | — | −0.16 |
| Ile | 188 | — | — | B | B | — | — | — | −1.02 |
| Ser | 189 | — | — | B | — | — | — | — | −1.37 |
| Leu | 190 | — | — | B | — | — | — | — | −0.51 |
| Gly | 191 | — | — | — | — | — | T | C | 0.41 |
| Pro | 192 | — | — | — | — | — | T | C | 0.71 |
| Gly | 193 | — | — | — | — | T | T | — | 0.74 |
| Asp | 194 | — | — | — | — | T | T | — | 0.46 |
| Arg | 195 | — | — | B | B | — | — | — | 1.02 |
| Asp | 196 | — | — | B | B | — | — | — | 1.07 |
| Val | 197 | — | — | B | B | — | — | — | 0.61 |
| Ala | 198 | — | — | B | B | — | — | — | 0.07 |
| Tyr | 199 | — | — | B | B | — | — | — | −0.79 |
| Ser | 200 | | — | B | B | | — | — | −1.20 |
| Cys | 201 | — | — | B | B | — | — | — | −1.20 |
| Ile | 202 | — | — | B | B | — | — | — | −0.56 |
| Val | 203 | — | | B | B | — | — | — | −0.82 |
| Ser | 204 | — | | B | B | | — | — | −0.88 |

| | | Eisen . . . | Eisen . . . | Karpl . . . | James . . . | Emini |
|---|---|---|---|---|---|---|
| Res | Pos | Alpha | Beta | Flexi . . . | Antig . . . | Surfa . . . |
| Asp | 171 | — | — | — | 0.60 | 0.46 |
| Phe | 172 | — | — | — | 1.00 | 0.56 |
| Gly | 173 | — | — | — | 0.70 | 0.87 |
| Met | 174 | — | — | — | 0.70 | 0.71 |
| Glu | 175 | — | — | — | 0.45 | 1.10 |
| Pro | 176 | — | — | F | 0.45 | 0.91 |
| His | 177 | — | — | — | 0.20 | 0.80 |
| Ser | 178 | — | — | — | 0.30 | 0.67 |
| Leu | 179 | — | — | — | −0.10 | 0.72 |
| Phe | 180 | — | — | — | −0.10 | 0.52 |
| Thr | 181 | — | — | F | 0.25 | 0.68 |
| Asp | 182 | — | — | F | 0.35 | 0.61 |
| Gly | 183 | — | — | F | −0.05 | 0.58 |
| Gln | 184 | — | — | F | 0.25 | 0.54 |
| Val | 185 | — | — | — | −0.30 | 0.23 |
| Leu | 186 | — | — | — | −0.60 | 0.31 |
| Ser | 187 | — | — | — | −0.60 | 0.15 |
| Ile | 188 | — | — | — | −0.60 | 0.19 |
| Ser | 189 | — | — | — | −0.06 | 0.36 |
| Leu | 190 | — | — | — | 0.28 | 0.27 |
| Gly | 191 | — | — | F | 1.47 | 0.64 |
| Pro | 192 | — | — | F | 2.71 | 0.94 |
| Gly | 193 | — | — | F | 3.40 | 1.90 |
| Asp | 194 | — | — | F | 3.06 | 1.42 |
| Arg | 195 | — | — | F | 1.77 | 0.93 |
| Asp | 196 | — | — | F | 1.58 | 1.47 |
| Val | 197 | — | — | — | 1.09 | 1.18 |
| Ala | 198 | — | — | — | 0.30 | 0.32 |
| Tyr | 199 | — | — | — | −0.60 | 0.14 |
| Ser | 200 | — | — | — | −0.60 | 0.14 |
| Cys | 201 | — | — | — | −0.60 | 0.18 |
| Ile | 202 | — | — | — | −0.60 | 0.18 |
| Val | 203 | — | — | — | −0.60 | 0.21 |
| Ser | 204 | — | — | F | −0.45 | 0.29 |

TABLE 1G

| | | Garni . . . | Chou- . . . | Garni . . . | Chou- . . . | Garni . . . | Chou- . . . | Garni . . . | Kyte- . . . |
|---|---|---|---|---|---|---|---|---|---|
| Res | Pos | Alpha | Alpha | Beta | Beta | Turn | Turn | Coil | Hydro . . . |
| Asn | 205 | — | — | B | — | — | T | — | −0.87 |
| Pro | 206 | — | — | B | — | — | T | — | −0.20 |
| Val | 207 | — | — | — | — | T | T | — | −0.12 |
| Ser | 208 | — | — | — | — | T | T | — | 0.14 |
| Trp | 209 | — | — | B | — | — | — | — | 0.13 |
| Asp | 210 | — | — | B | — | — | — | — | −0.72 |
| Leu | 211 | — | — | B | B | — | — | — | −0.82 |
| Ala | 212 | — | — | B | B | — | — | — | −0.18 |
| Thr | 213 | — | — | B | B | — | — | — | −0.17 |
| Val | 214 | — | — | B | B | — | — | — | 0.12 |
| Thr | 215 | — | — | — | B | — | — | C | −0.18 |
| Pro | 216 | — | — | — | B | T | — | — | −0.03 |
| Trp | 217 | — | — | — | — | T | T | — | 0.52 |
| Asp | 218 | — | — | — | — | T | T | — | 0.80 |
| Ser | 219 | — | — | — | — | T | T | — | 1.66 |
| Cys | 220 | — | — | B | — | — | T | — | 1.38 |
| His | 221 | — | A | B | — | — | — | — | 1.00 |
| His | 222 | — | A | — | — | — | — | C | 1.08 |
| Glu | 223 | — | A | — | — | — | — | C | 0.73 |
| Ala | 224 | — | A | — | — | — | — | C | 1.08 |
| Ala | 225 | — | — | — | — | — | T | C | 1.16 |
| Pro | 226 | — | — | — | — | T | T | — | 0.89 |
| Gly | 227 | — | — | — | — | T | T | — | 0.68 |
| Lys | 228 | — | — | — | — | T | T | — | 0.72 |
| Ala | 229 | — | — | — | — | — | T | C | 1.31 |
| Ser | 230 | — | — | B | — | — | T | — | 1.04 |
| Tyr | 231 | — | — | B | — | — | T | — | 0.44 |
| Lys | 232 | — | — | B | — | — | T | — | −0.02 |
| Asp | 233 | — | — | B | B | — | — | — | −0.92 |
| Val | 234 | — | — | B | B | — | — | — | −1.19 |
| Leu | 235 | — | — | B | B | — | — | — | −1.74 |
| Leu | 236 | — | — | B | B | — | — | — | −1.71 |
| Val | 237 | — | — | B | B | — | — | — | −2.61 |
| Val | 238 | — | — | B | B | — | — | — | −2.91 |

| | | Eisen . . . | Eisen . . . | Karpl . . . | James . . . | Emini |
|---|---|---|---|---|---|---|
| Res | Pos | Alpha | Beta | Flexi . . . | Antig . . . | Surfa . . . |
| Asn | 205 | — | — | F | −0.05 | 0.56 |
| Pro | 206 | — | — | F | −0.05 | 0.80 |
| Val | 207 | — | — | F | 0.65 | 1.00 |
| Ser | 208 | — | — | — | 0.20 | 0.51 |
| Trp | 209 | — | — | — | −0.40 | 0.33 |
| Asp | 210 | — | — | — | −0.40 | 0.65 |
| Leu | 211 | — | — | — | −0.60 | 0.36 |
| Ala | 212 | — | — | — | −0.60 | 0.49 |
| Thr | 213 | — | — | — | −0.30 | 0.46 |
| Val | 214 | — | — | — | −0.60 | 0.58 |
| Thr | 215 | — | — | F | −0.25 | 0.96 |
| Pro | 216 | — | — | F | 0.25 | 0.89 |
| Trp | 217 | — | — | F | 0.35 | 0.65 |
| Asp | 218 | — | — | F | 0.65 | 0.61 |
| Ser | 219 | — | — | — | 0.50 | 0.54 |
| Cys | 220 | — | — | — | 0.70 | 0.88 |
| His | 221 | — | — | — | 0.60 | 0.53 |
| His | 222 | — | — | — | 0.81 | 0.40 |
| Glu | 223 | — | — | — | 0.67 | 1.16 |
| Ala | 224 | — | — | — | 1.43 | 0.84 |
| Ala | 225 | — | — | F | 2.74 | 1.24 |
| Pro | 226 | — | — | F | 3.10 | 0.72 |
| Gly | 227 | — | — | F | 2.49 | 0.96 |
| Lys | 228 | — | — | F | 1.90 | 1.49 |
| Ala | 229 | — | — | F | 2.16 | 1.92 |
| Ser | 230 | — | — | F | 2.12 | 3.25 |
| Tyr | 231 | — | — | F | 1.98 | 1.21 |
| Lys | 232 | — | — | F | 1.70 | 0.98 |
| Asp | 233 | — | — | — | 0.38 | 0.61 |
| Val | 234 | — | — | — | −0.09 | 0.29 |
| Leu | 235 | — | — | — | 0.04 | 0.11 |
| Leu | 236 | — | — | — | −0.43 | 0.05 |
| Val | 237 | — | — | — | −0.60 | 0.10 |
| Val | 238 | — | — | — | −0.60 | 0.09 |

TABLE 1H

| | | Garni . . . | Chou- . . . | Garni . . . | Chou- . . . | Garni . . . | Chou- . . . | Garni . . . | Kyte- . . . |
|---|---|---|---|---|---|---|---|---|---|
| Res | Pos | Alpha | Alpha | Beta | Beta | Turn | Turn | Coil | Hydro . . . |
| Val | 239 | — | — | B | B | — | — | — | −2.87 |
| Pro | 240 | — | — | B | B | — | — | — | −2.87 |
| Val | 241 | — | — | B | B | — | — | — | −2.87 |
| Ser | 242 | — | — | B | B | — | — | — | −2.61 |
| Leu | 243 | — | — | B | B | — | — | — | −2.57 |
| Leu | 244 | — | — | B | B | — | — | — | −2.57 |
| Leu | 245 | — | — | B | B | — | — | — | −2.67 |
| Met | 246 | — | — | B | B | — | — | — | −2.62 |
| Leu | 247 | — | — | B | B | — | — | — | −3.02 |
| Val | 248 | — | — | B | B | — | — | — | −2.51 |
| Thr | 249 | — | — | B | B | — | — | — | −2.29 |
| Leu | 250 | — | — | B | B | — | — | — | −1.77 |
| Phe | 251 | — | — | B | B | — | — | — | −1.20 |
| Ser | 252 | — | — | — | B | T | — | — | −0.68 |
| Ala | 253 | — | — | — | B | T | — | — | −0.49 |
| Trp | 254 | — | — | — | B | T | — | — | −0.39 |
| His | 255 | — | — | — | B | T | — | — | −0.24 |
| Trp | 256 | — | — | — | B | T | — | — | 0.16 |
| Cys | 257 | — | — | B | — | — | T | — | 0.11 |
| Pro | 258 | — | — | — | — | T | T | — | 0.74 |
| Cys | 259 | — | — | — | — | T | T | — | 1.08 |
| Ser | 260 | — | — | — | — | T | T | — | 1.16 |
| Gly | 261 | — | — | — | — | T | T | — | 1.49 |
| Lys | 262 | — | — | — | — | T | T | — | 2.16 |
| Lys | 263 | — | — | — | — | T | T | — | 1.51 |
| Lys | 264 | — | — | — | — | T | T | — | 2.14 |
| Lys | 265 | — | A | B | — | — | — | — | 1.86 |
| Asp | 266 | — | A | B | — | — | — | — | 2.20 |
| Val | 267 | — | A | B | — | — | — | — | 2.27 |
| His | 268 | — | A | B | — | — | — | — | 1.37 |
| Ala | 269 | — | A | B | — | — | — | — | 0.98 |
| Asp | 270 | — | A | B | — | — | — | — | 0.72 |
| Arg | 271 | | A | B | — | — | — | — | 0.72 |
| Val | 272 | — | A | — | — | — | — | C | 1.27 |

| | | Eisen . . . | Eisen . . . | Karpl . . . | James . . . | Emini |
|---|---|---|---|---|---|---|
| Res | Pos | Alpha | Beta | Flexi . . . | Antig . . . | Surfa . . . |
| Val | 239 | — | — | — | −0.60 | 0.14 |
| Pro | 240 | — | — | — | −0.60 | 0.16 |
| Val | 241 | — | — | — | −0.60 | 0.18 |
| Ser | 242 | — | — | — | −0.60 | 0.20 |
| Leu | 243 | — | — | — | −0.60 | 0.13 |
| Leu | 244 | — | — | — | −0.60 | 0.14 |
| Leu | 245 | — | — | — | −0.60 | 0.08 |
| Met | 246 | — | — | — | −0.60 | 0.14 |
| Leu | 247 | — | — | — | −0.60 | 0.14 |
| Val | 248 | — | — | — | −0.60 | 0.14 |
| Thr | 249 | — | — | — | −0.60 | 0.19 |
| Leu | 250 | — | — | — | −0.60 | 0.24 |
| Phe | 251 | — | — | — | −0.60 | 0.34 |
| Ser | 252 | — | — | — | −0.20 | 0.32 |
| Ala | 253 | — | — | — | −0.20 | 0.40 |
| Trp | 254 | — | — | — | −0.20 | 0.25 |
| His | 255 | — | — | — | −0.20 | 0.29 |
| Trp | 256 | — | — | — | −0.20 | 0.15 |
| Cys | 257 | — | — | — | 0.14 | 0.20 |
| Pro | 258 | — | — | — | 0.88 | 0.14 |
| Cys | 259 | — | — | — | 1.52 | 0.27 |
| Ser | 260 | — | — | F | 3.06 | 1.01 |
| Gly | 261 | — | — | F | 3.40 | 1.30 |
| Lys | 262 | — | — | F | 3.06 | 4.86 |
| Lys | 263 | — | — | F | 2.72 | 6.06 |
| Lys | 264 | — | — | F | 2.38 | 4.54 |
| Lys | 265 | — | — | F | 1.24 | 3.09 |
| Asp | 266 | — | — | F | 0.90 | 1.56 |
| Val | 267 | — | — | — | 0.75 | 1.30 |
| His | 268 | — | — | — | 0.75 | 1.28 |
| Ala | 269 | — | — | — | 0.90 | 0.57 |
| Asp | 270 | — | — | — | 0.90 | 0.76 |
| Arg | 271 | — | — | F | 1.35 | 0.86 |
| Val | 272 | — | — | F | 2.30 | 1.47 |

TABLE 1I

| | | Garni . . . | Chou- . . . | Garni . . . | Chou- . . . | Garni . . . | Chou- . . . | Garni . . . | Kyte- . . . |
|---|---|---|---|---|---|---|---|---|---|
| Res | Pos | Alpha | Alpha | Beta | Beta | Turn | Turn | Coil | Hydro . . . |
| Gly | 273 | — | — | — | — | — | T | C | 1.30 |
| Pro | 274 | — | — | — | — | — | T | C | 1.89 |
| Glu | 275 | — | — | — | — | — | T | C | 1.68 |
| Thr | 276 | — | — | — | — | — | T | C | 0.76 |
| Glu | 277 | — | — | — | — | — | — | C | 0.76 |
| Asn | 278 | — | — | B | — | — | T | — | 1.10 |
| Pro | 279 | — | — | B | — | — | T | — | 1.31 |
| Leu | 280 | — | — | B | — | — | T | — | 0.50 |
| Val | 281 | — | — | B | — | — | T | — | 0.60 |
| Gln | 282 | — | — | B | — | — | — | — | 0.21 |
| Asp | 283 | — | — | B | — | — | — | — | −0.18 |
| Leu | 284 | — | — | B | — | — | — | — | −0.36 |
| Pro | 285 | — | — | B | — | — | — | — | 0.07 |
| Ter | 286 | — | — | B | — | — | — | — | 0.53 |

| | | Eisen . . . | Eisen . . . | Karpl . . . | James . . . | Emini |
|---|---|---|---|---|---|---|
| Res | Pos | Alpha | Beta | Flexi . . . | Antig . . . | Surfa . . . |
| Gly | 273 | — | — | F | 3.00 | 1.27 |
| Pro | 274 | — | — | F | 2.70 | 1.13 |
| Glu | 275 | — | — | F | 2.40 | 2.44 |
| Thr | 276 | — | — | F | 2.10 | 3.82 |
| Glu | 277 | — | — | F | 1.60 | 2.03 |
| Asn | 278 | — | — | F | 0.85 | 0.87 |
| Pro | 279 | — | — | F | 0.40 | 1.05 |
| Leu | 280 | — | — | F | 1.00 | 1.01 |
| Val | 281 | — | — | F | 0.38 | 0.52 |
| Gln | 282 | — | — | F | 0.31 | 0.52 |
| Asp | 283 | — | — | F | 0.44 | 0.80 |
| Leu | 284 | — | — | — | 0.57 | 1.38 |
| Pro | 285 | — | — | — | 1.30 | 1.02 |
| Ter | 286 | — | — | — | 1.02 | 0.78 |

What is claimed is:

1. An isolated nucleic acid molecule comprising a polynucleotide having a nucleotide sequence at least 95% identical to a sequence selected from the group consisting of:
(a) a polynucleotide fragment of SEQ ID NO:1 or a polynucleotide fragment of the cDNA sequence included in ATCC™ Deposit No: 209623;
(b) a polynucleotide encoding a polypeptide fragment of SEQ ID NO:2 or the cDNA sequence included in ATCC™ Deposit No: 209623;
(c) a polynucleotide encoding a polypeptide domain of SEQ ID NO:2 or the cDNA sequence included in ATCC™ Deposit No: 209623;
(d) a polynucleotide encoding a polypeptide epitope of SEQ ID NO:2 or the cDNA sequence included in ATCC™ Deposit No: 209623;
(e) a polynucleotide encoding a polypeptide of SEQ ID NO:2 or the cDNA sequence included in ATCC™ Deposit No: 209623 having biological activity;
(f) a polynucleotide which is a variant of SEQ ID NO:1;
(g) a polynucleotide which is an allelic variant of SEQ ID NO:1;
(h) a polynucleotide which encodes a species homologue of the SEQ ID NO:2;
(i) a polynucleotide capable of hybridizing under stringent conditions to any one of the polynucleotides specified in (a)-(h), wherein said polynucleotide does not hybridize under stringent conditions to a nucleic acid molecule having a nucleotide sequence of only A residues or of only T residues.

2. The isolated nucleic acid molecule of claim 1, wherein the polynucleotide fragment comprises a nucleotide sequence encoding a mature form or a secreted protein.

3. The isolated nucleic acid molecule of claim 1, wherein the polynucleotide fragment comprises a nucleotide sequence encoding the sequence identified as SEQ ID NO:2 or the coding sequence included in ATCC™ Deposit No: 209623.

4. The isolated nucleic acid molecule of claim 1, wherein the polynucleotide fragment comprises the entire nucleotide sequence of SEQ ID NO:1 or the cDNA sequence included in ATCC™ Deposit No: 209623.

5. A recombinant vector comprising the isolated nucleic acid molecule of claim 1.

6. A method of making a recombinant host cell comprising the isolated nucleic acid molecule of claim 1.

7. A recombinant host cell produced by the method of claim 6.

8. The recombinant host cell of claim 7 comprising vector sequences.

9. An isolated polypeptide comprising an amino acid sequence at least 95% identical to a sequence selected from the group consisting of:
(a) a polypeptide fragment of SEQ ID NO:2 or the encoded sequence included in ATCC™ Deposit No: 209623;
(b) a polypeptide fragment of SEQ ID NO:2 or the encoded sequence included in ATCC™ Deposit No: 209623 having biological activity;
(c) a polypeptide domain of SEQ ID NO:2 or the encoded sequence included in ATCC™ Deposit No: 209623;
(d) a polypeptide epitope of SEQ ID NO:2 or the encoded sequence included in ATCC™ Deposit No: 209623;
(e) a mature form of a secreted protein;
(f) a full length secreted protein;
(g) a variant of SEQ ID NO:2;
(h) an allelic variant of SEQ ID NO:2; or
(i) a species homologue of the SEQ ID NO:2.

10. An isolated antibody that binds specifically to the isolated polypeptide of claim 9.

11. A recombinant host cell that expresses the isolated polypeptide of claim 9.

12. A method of making an isolated polypeptide comprising:
(a) culturing the recombinant host cell of claim 11 under conditions such that said polypeptide is expressed; and
(b) recovering said polypeptide.

13. The polypeptide produced by claim 12.

14. A method for preventing, treating, or ameliorating a medical condition which comprises administering to a mammalian subject a therapeutically effective amount of the polypeptide of claim 9.

15. A method of diagnosing a pathological condition or a susceptibility to a pathological condition in a subject related to expression or activity of a secreted protein comprising:
(a) determining the presence or absence of a mutation in the polynucleotide of claim 1; and
(b) diagnosing a pathological condition or a susceptibility to a pathological condition based on the presence or absence of said mutation.

16. A method of diagnosing a pathological condition or a susceptibility to a pathological condition in a subject related to expression or activity of a secreted protein comprising:
(a) determining the presence or amount of expression of the polypeptide of claim 9 in a biological sample; and
(b) diagnosing a pathological condition or a susceptibility to a pathological condition based on the presence or amount of expression of the polypeptide.

17. A method for identifying binding partner to the polypeptide of claim 9 comprising:
(a) contacting the polypeptide of claim 9 with a binding partner; and
(b) determining whether the binding partner effects an activity of the polypeptide.

18. A method of identifying an activity in a biological assay, wherein the method comprises:
(a) expressing SEQ ID NO:1 in a cell;
(b) isolating the supernatant;
(c) detecting an activity in a biological assay; and
(d) identifying the protein in the supernatant having the activity.

19. The product produced by the method of claim 18.
